SEQUENCE LISTING (45 sequences)

SEQ ID NO: 1 - protein, 156 aa, Pseudomonas putida
MSQVTNTPYEALEVGQKAEYKKSVEERDIQLFAAMSGDHNPVHLDAEFAAKSMFRERIAHGMFSGALISAAVACTLPGPGTIYLGQQMSFQKPVKIGDTLTVRLEILEKLPKFKVRIATNVYNQNDELVVAGEAEILAPRKQQTVELVSPPNFVAS

SEQ ID NO: 2 - protein, 151 aa, Pseudomonas putida
MPHVPVTELSQYVGKELGHSEWLKIDQQRINLFAEATGDFQFIHVDPEKAAKTPFGGTIAHGFLTLSLIPKLIEDILVLPQGLKMVVNYGLDSVRFIQPVKVDSRVRLKVKLGEVVEKKPGQWLLKAIATLEIEGEEKPAYIAESLSLCFV

SEQ ID NO: 3 - protein, 285 aa, Pseudomonas aeruginosa
MPTAWLDLPAPPALPGLFLRAALRRGIRGKALPERGLRSQVTVDPKHLERYRQVCGFRDDGLLPPTYPHILAFPLQMALLTDKRFPFPLLGLVHLENRIDVLRALGGLGPFTVSVAVENLQPHDKGATFSIVTRLEDQLGLLWVGDSKVLCRGVKVPGEIPPKAEQEPLPLEPVDNWKAPADIGRRYARAAGDYNPIHLSAPSAKLFGFPRAIAHGLWNKARSLAALGERLPASGYRVEVRFQKPVLLPASLTLLASAAAADGQFSLRGKDDLPHMAGHWSRLQG

SEQ ID NO: 4 - protein, 151 aa, Pseudomonas aeruginosa
MPFVPVAALKDYVGKDLGHSEWLTIDQERVDQFAECTGDHQFIHVDPEKAAKTPFGGTIAHGFLSLSLIPKLMEGLLVLPEGLKMAVNYGLDTVRFIQPVRVGSRVRLGLTLLDVNEKNPGQWLIKARATLEIEGQEKPAYIAETLSLCFV

SEQ ID NO: 5 - protein, 152 aa, Pseudomonas citronellolis
MPRVFEHPEQLLACVGETLGESEWLSVEQSRIDRFAEATGDHQWIHVDPVRAASGPYGACIAHGYLTLALVNLFLPQIVEVRGIAMGVNYGCDRVRFPNVVKAGARVRGRAELVAAEAVKGAVQATIRVSVEIDGEDRPGCVVDTISRYYPA

SEQ ID NO: 6 - protein, 151 aa, Pseudomonas mendocina
MPSVPVAELKDYVGKELGKSEWLTIDQQRINQFAECTGDHQFIHVDPEKAKLTPFGTTIAHGFLSLSLVPMLMEKIMIMPQGLKMAVNYGLDSVRFIQPVKVNSKVRLVVTLTDANEKNPGQWLLKARAVLEIEGQEKPAYIAEPLTLCFV

SEQ ID NO: 7 - protein, 283 aa, Pseudomonas putida
MSRQWHDLHNPAARASLYLRAASKRSISGERLPDDGLRCFIRVQPGNLGAYRRLCHFPDDGRLPGTYPHVMAFALQLQLMTADDFPFPLLGLVHLHNRIEVLRPLGAVEGLRFAVYTHNLQRHAKGGTFDLVTEAEDGIGLLWRETSRMLVRGLALEGEASEPADPEPDPLAEATRWYADSDIGRRYAKVCGDYNPIHLSAVSARLFGFPTAIAHGMWTKAMALAALRGHLPHSGYAFEVDFRKPVRLPSEVVLSASEAGPSGQLRLDGHGDVLHMVGRWESL

SEQ ID NO: 8 - protein, 293 aa, Pseudomonas plecoglossicida
MRPGLDGGLSMSRHWHDLHSPASRTSLYLRAASKRSISGDQLPDDGLRCFIRVQPGNLAAYRRLCHFTDDGRLPGTYPHVMAFTLQLQLMTAPNFPFPLLGLVHLHNRIEVLRPLGGIEGLRFAVHAGNLQAHAKGGTFDLVTEAEDGIGLLWRETSRMLVRGLKLESQAGEPAEDEPQALPEATRWYADSDIGRRYAKVCGDYNPIHLSAASARLFGFPTAIAHGMWSKAMALAALRGHLPHSGFAFEVDFRKPVRLPSEVVLGASEAGTTGELRLDGHGGVLHMVGRWDRL

SEQ ID NO: 9 - protein, 151 aa, Pseudomonas plecoglossicida
MPHVPVTELSQYVGRELGRSAWLKIDQQRINLFAEATGDFQFIHVDPEKAAKTPFGSTIAHGFLTLSLIPKLIEDILVLPEGLKMVVNYGLDSVRFIQPVKVDSQVRLKVDLAEVTEKRPGQWLLKAIATLEIDGEEKPAFIAESLSLCFV

SEQ ID NO: 10 - protein, 151 aa, Pseudomonas mosselii
MPYVPVTELSQYVGKELGRSQWLKIDQERINLFAEATGDFQFIHVDPVKAAKTPFGSTIAHGFLTLSLIPKLMEDILVLPEGLKMVVNYGLDSVRFIQPVKVDSQVRLKVDLTDATEKKPGQWLLKATVTLEIEGEEKPAYIAEPLSLCFV

SEQ ID NO: 11 - protein, 151 aa, Pseudomonas plecoglossicida
MPLVPVTELSQYVGKELGCSDWLKIDQERINLFAEATGDFQFIHVDPVKAAKTPFGCTIAHGFLTLSLISKLIEDVVVLPEGNKMVVNYGLDSVRFIQPVKVDSKVRLKVELTEATEKKPGQWLLKSKVALEIEGEEKPAYIAEPLILCFV

SEQ ID NO: 12 - protein, 151 aa, Pseudomonas fulva
MPLVPVTALTQYVGKSLGHSAWLTIDQQRIDRFAEATGDHQFIHVDPQRAAQTAFGGTIAHGFLTLSLIPTLIEDILVLPQGLKMVVNYGLDSVRFIQPVKVDSQVRLHVELADVQQKRPGQWLLKAIATLEIKDEDKPAYRAESLSLCFV

SEQ ID NO: 13 - protein, 151 aa, Pseudomonas fulva
MPLVPVTALTQYIGKSLGHSAWLTIDQQRIDRFAEATGDHQFIHVDPQRAAQTVFGCTIAHGFLTLSLIPALIEDILVLPQGLKMVVNYGLGSVRFIQPVKVDSQVRLHVELADVQQKRPGQWLLKAIATLEIKDEDKPAYRAESLSLCFV

SEQ ID NO: 14 - protein, 397 aa, Treponema denticola
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRI

SEQ ID NO: 15 - protein, 539 aa, Euglena gracialis
MSCPASPSAAVVSAGALCLCVATVLLATGSNPTALSTASTRSPTSLVRGVDRGLMRPTTAAALTTMREVPQMAEGFSGEATSAWAAAGPQWAAPLVAAASSALALWWWAARRSVRRPLAALAELPTAVTHLAPPMAMFTTTAKVIQPKIRGFICTTTHPIGCEKRVQEEIAYARAHPPTSPGPKRVLVIGCSTGYGLSTRITAAFGYQAATLGVFLAGPPTKGRPAAAGWYNTVAFEKAALEAGLYARSLNGDAFDSTTKARTVEAIKRDLGTVDLVVYSIAAPKRTDPATGVLHKACLKPIGATYTNRTVNTDKAEVTDVSIEPASPEEIADTVKVMGGEDWELWIQALSEAGVLAEGAKTVAYSYIGPEMTWPVYWSGTIGEAKKDVEKAAKRITQQYGCPAYPVVAKALVTQASSAIPVVPLYICLLYRVMKEKGTHEGCIEQMVRLLTTKLYPENGAPIVDEAGRVRVDDWEMAEDVQQAVKDLWSQVSTANLKDISDFAGYQTEFLRLFGFGIDGVDYDQPVDVEADLPSAAQQ

SEQ ID NO: 16 - protein, 400 aa, Enterovibrio corallii
MIIKPVIQGVVARSAHPLGCQQAVLNQIRYVQAAPQISGGPKKVLVLGASSGFGLASRISLAFGGAKADTIGISFEKGPSEKGAGTAGWYNNVFFRQEAEKHGLIAKNFVGDAFSSEMRKQVIDYIKKEFGGHVDLVVYSLATGIRPNPETGELWRSSIKTLDKPLSGPTIDLERDVMLPLDVGVATEDELDATVKVMGGEDWESWIAVLSDAGVLAKGCKTLAYSYIGPEFTYPIYHRGTLGKAKDHLHKTADVLNQTLQDLEGEAYVAVCKALVTKASVFIPAFSPYILSLFKVMKENGTHEGCIEQMQRLFTERLYRNGDHVPVDNARLIRMDDWELAPETQSAVAALMAEMTADNFQHVGDYQGYKQDFLQLNGFDFAGIDYDMPVDMAFLMSLRP

SEQ ID NO: 17 - protein, 398 aa, Lactobacillus oligofermentans
MKIDKKFKGNVARSVNPYGCHQEVLNQIKYVKENGHYDGAKKALIIGGSSSYGLASRITQAFGSGADTISVAFERPIKDETMLGTAGWWNNIYFKQEAEKAGLIAKNFNGDCFTEKMKEQVSEYIKDEFGGKIDLLVYSVAAPKRANPENTDEVWRSQMKPIGKDVTGYNVNLEQDALVEQTIEAATPEEVADTVHVMGGEDWELWVKFLKDQGLLADGFKTILYSYIGSPVTYDFYHEGTLGKAKDAAEESSHRIGKIIEDINGESLISVSKAVTTKASSVIPIFPVYCIALYKVMQEKGTHETPIMHKDRLFRDMVYGNKREIDDHGRLRPDSWELDEDTQRKTLDLMNKITPENFNTDLTGYETFKKEFLQINGFEVEGSNNETVDFDEISKLEP

SEQ ID NO: 18 - protein, 403 aa, Paucilactobacillus oligofermentans
MLGKMIKIDKKFKGNVARSVNPYGCHQEVLNQIKYVKENGHYDGAKKALIIGGSSSYGLASRITQAFGSGADTISVAFERPIKDETMLGTAGWWNNIYFKQEAEKAGLIAKNFNGDCFTEKMKEQVSEYIKDEFGGKIDLLVYSVAAPKRANPENTDEVWRSQMKPIGKDVTGYNVNLEQDALVEQTIEAATPEEVADTVHVMGGEDWELWVKFLKDQGLLADGFKTILYSYIGSPVTYDFYHEGTLGKAKDAAEESSHRIGKIIEDINGESLISVSKAVTTKASSVIPIFPVYCIALYKVMQEKGTHETPIMHKDRLFRDMVYGNKREIDDHGRLRPDSWELDEDTQRKTLDLMNKITPENFNTDLTGYETFKKEFLQINGFEVEGSNNETVDFDEISKLEP

SEQ ID NO: 19 - protein, 400 aa, Vibrio harveyi
MIIKPRIRGFICTTTHPVGCEANVKEQIAYTKAQGPIKNAPKRVLVVGASSGYGLSSRIAAAFGGGASTIGVFFEKEGTEKKPGTAGFYNSVAFEKLAREEGLYAKSLNGDAFSNEAKQKTIDLIKEDLGQVDMVVYSLASPVRKMPETGELIRSALKPIGETYTSTAVDTNKDVIIEASVEPATEQEIQDTVTVMGGEDWELWINALSEAGVLADGCKTVAYSYIGTELTWPIYWDGALGKAKMDLDRAAAALNEKLSTTGGSANVAVLKSVVTQASSAIPVMPLYIAMVFKKMREEGVHEGCMEQIFRMFSQRLYKEDGSAAEVDEVNRLRLDDWELREDIQQHCRDLWPQITSENLKELTDYVEYKEEFLKLFGFGVDGVDYEADVNPAVETDFTQI

SEQ ID NO: 20 - protein, 394 aa, Treponema lecithinolyticum
MIIKPMVRGNICLNAHPAGCKKSVEDQIAYTKKGGKRTSALKNALILGCSNGYGLASRITASFGYGAATIGVSFEKAGSETKWGTPGWYNNLAFDQAAKEAGLTAVTIDGDAFSDGIKETVIGEAKNRGMKFDLVVYSLASPVRVDPDTGVMHKSVLKPFGKAFCGKTLDPFTGKLSEITAEPATDEEAAATVKVMGGEDWQRWMDKLYKAGVLADGCITVAYSYIGPDATQALYRKGTIGKAKEHLEATAHTLNKEMAAFGGKAFVSVNKGLVTRASAVIPVIPLYLASLFKVMKQKGNHEGCIEQMNRLFDERLYRSDKQIPVDSENRIRIDDWELSDDVQSAVRALMEQVTDENSEKLTDLEGYRHDFLAANGFDIAGVDYQADIERFDRL

SEQ ID NO: 21 - protein, 398 aa, Treponema pedis
MIIKPMVRGNVCLNSHPKGCKKAVEHQIAYTKKRSADFSSGTVKPKNALILGCSNGYGLASRITAAFGYGAATIGVSFEKAGSETKWGTPGWYNNLAFDGAAKAEGLVSITIEGDAFSDAVKAEVIKKAKEHGMNFDLVIYSLASPLRIDPDTGVTYKSVLKPFGKAFTGKTLDPFTGKLSEITAEPATEEEAAATVKVMGGEDWQRWIEQLSEAGVLAEGCITAAYSYIGPEATQALYRKGTIGKAKEHLEKTAHILNDKMKTFNGKAFVSVNKGLVTRASAVIPVIPLYLASLFKVMKEKGNHEGCIEQINRLFSERLYTKDGKIPVDGENRIRIDDWELDDEVQAAVDKIMEKVTDDNSETLTDLAGYRHDFLAANGFDIDGIDYDCDVERCDGI

SEQ ID NO: 22 - protein, 398 aa, Treponema vincentii
MSMKPMLRSNICLNAHPQGCKKAVEDQIAYTKKRAKSHPAGTAVPKNVLIVGCSGGYGLASRITAAFGYGATTIGVSYEKAGSEKKWGTPGWYNNLAVDTAAKEAGLVSVTIDGDAFSDAIKTQVIDEAKKLGIKFDLIIYSVASSVRTDPETGVTYRSALKPFGKPFTGKTLDPFTGALTEITAEPATDEEAFATVKVMGGEDWQRWIEKLGAANLLAQGCITVAYSYIEPEATQALYRKGTIGKAKEHLEATAHALNTKLAALKGQAFVSVNKGLVTRASAVIPVIPLYLASLFKVMKEKGTHEGCIEQINRLFDSRLYTTDGVIPTDSEHRIRIDDWELDESVQSAVAEIMATVTDETSRERTDVDEYRHDFLAINGFDIAGIDYDAEIDRFDRI

SEQ ID NO: 23 - protein, 410 aa, Treponema sp.
MVIKPMIRSNICLNAHPAGCKKAVEDQIEYVKAQAAKRAETGKSVLKSSKYAPKNVLVLGCSNGYGLASRITAAFGYGASTIGVSFEKAGSETKWGTPGWYNNLAFDAAAKKENIKSITIDGDAFSHAVKQQVVDQAKAQNMKFDLVVYSLASPVRVDPDTNVMYKSVLKPIGKVFSGMTIDPFTGELKEISAEPANEEEIINTQKVMGGEDWALWIEKLEKENVLAENCITLAYSYVGPEVSHAIYRSGTIGKAKEHLEKTAQELTKKMASYGGRAYVSVNKGLVTRASAVIPIIPLYLSVLFKVMKAKGTHEGCIEQIDRLFAERLYISEDRTKTPIPVDEENRIRIDDLEMDSEVQAEVSRIMPTVTSENSAELTDLEGYRHDFLATSGFDIDGVDYEADIEAFDRI

SEQ ID NO: 24 - protein, 403 aa, Treponema brennaborense
MIIKPMIRSNICINAHPVGCAKETERQIAYVQSRKANRGTKTAAEGGKAPKAVLVLGCSTGYGLASRITAAFEYGAATIGVSFEKEGSEKKGGTPGWYNNLAFDRASKKAGIPSVTLNMDAFSDDCRKAVVAEARKMGVAFDLVVYSLASPVRTDPETGVLYKSVLKPFGKPFSGQTVDMMSGELSVMSAEPANDDEAAQTVKVMGGEDWERWIDQLAEAGVLAKGCKTVAYSYIGPALSHAIYRDGTIGGAKKHLEKTARALDSKLSQKLGGEAFVSVNKGLVTRSSAVIPIIPLYLSVLFKVMKEKGTHEGCIEQMERLFAERLYTDGSVPVDAEHLIRMDDWELDPAVQAEVDKRLAQVTQDTLASLGDMDGYRHDFLATNGFDVAGVDYDADVERMDVV

SEQ ID NO: 25 - protein, 409 aa, Treponema parvum
MVIKPMVRSNICINAHPVGCAKETENQIEYVKAQKKKRGIKNVKEGGKGPKTVLVLGCSTGYGLASRIVSAFAYGADTIGVSYEKEATETKGGTPGWYNNTAFDAAAKKAGLVSITINADAYADETRSQVIGEAKKLGRKFDLIIYSLASPVRTDPDTKVLYKSVIKPRGKPYSGKYIDIMSQTLKESSEEPATEEEIANTVKVMGGDDWRRWIKQLSEADVLAKGCRTVAYSYIGPELSHAIYRDGTIGTAKLDLEKAALDLNEELKSSVGGGAFISVNKGLVTRSSAVIPIISLYLSILFKVMKEKGTHEGCIEQIERLFAERLYTGADNSASDVPTDSEHRIRIDDWELADDVQKRCRERMAAVTQENLSQLCDLEGYKHDFLATNGFDIAGVDYDKDVARMDSIS

SEQ ID NO: 26 - protein, 814 aa, Escherichia coli [sequence omitted]

SEQ ID NO: 27 - protein, 561 aa, Escherichia coli
MKKVWLNRYPADVPTEINPDRYQSLVDMFEQSVARYADQPAFVNMGEVMTFRKLEERSRAFAAYLQQGLGLKKGDRVALMMPNLLQYPVALFGILRAGMIVVNVNPLYTPRELEHQLNDSGASAIVIVSNFAHTLEKVVDKTAVQHVILTRMGDQLSTAKGTVVNFVVKYIKRLVPKYHLPDAISFRSALHNGYRMQYVKPELVPEDLAFLQYTGGTTGVAKGAMLTHRNMLANLEQVNATYGPLLHPGKELVVTALPLYHIFALTINCLLFIELGGQNLLITNPRDIPGLVKELAKYPFTAITGVNTLFNALLNNKEFQQLDFSSLHLSAGGGMPVQQVVAERWVKLTGQYLLEGYGLTECAPLVSVNPYDIDYHSGSIGLPVPSTEAKLVDDDDNEVPPGQPGELCVKGPQVMLGYWQRPDATDEIIKNGWLHTGDIAVMDEEGFLRIVDRKKDMILVSGFNVYPNEIEDVVMQHPGVQEVAAVGVPSGSSGEAVKIFVVKKDPSLTEESLVTFCRRQLTGYKVPKLVEFRDELPKSNVGKILRRELRDEARGKVDNKA

SEQ ID NO: 28 - protein, 382 aa, Umbellularia californica
MATTSLASAFCSMKAVMLARDGRGMKPRSSDLQLRAGNAPTSLKMINGTKFSYTESLKRLPDWSMLFAVITTIFSAAEKQWTNLEWKPKPKLPQLLDDHFGLHGLVFRRTFAIRSYEVGPDRSTSILAVMNHMQEATLNHAKSVGILGDGFGTTLEMSKRDLMWVVRRTHVAVERYPTWGDTVEVECWIGASGNNGMRRDFLVRDCKTGEILTRCTSLSVLMNTRTRRLSTIPDEVRGEIGPAFIDNVAVKDDEIKKLQKLNDSTADYIQGGLTPRWNDLDVNQHVNNLKYVAWVFETVPDSIFESHHISSFTLEYRRECTRDSVLRSLTTVSGGSSEAGLVCDHLLQLEGGSEVLRARTEWRPKLTDSFRGISVIPAEPRV

SEQ ID NO: 29 - protein, 508 aa, Limnobacter sp.
MARNIPLLDASWLYVESKEAPMHVGSMAIFTVPEGETSQQAIARIVQMLRNSLEFAPPFNYRLSSPRLLTLMPKWIEADKIDLDYHFRHSALPAPGGERELGTLISRLHSHPLDFRKPLWEMHLIEGLYGNRFALYTKMHHSLMDGVGGMRLMERIFGKSAKESMNLPAPWSVGTISRKKKNSEPQHFADQAREAWEAAKLSGQSLPAAGRALMDLMREAVKPTDPALATPFSGPKSIVNKRVGGARRLATQTYPLERVRAVAEAAKVSVNDIFLAICSSSIRRYLLERDALPSESLTAGLPVSVRPADDLDGGNAISFIIANLYTTEADPLTRLKEIRRSTQLAKANLQAMPKEAINNYTIMLMAPMMLQLVSGLGGLTRPIFNTVISNVPGPSRDLYFSGCRLEQFYPISLIPHGQALNITVVSYSGQFNVAFTGDHDALPSMQRLSVYTGEALEELEAALGVKWASKPVVKPVTEKRPVAAKKPAVRKPATAKVGAGKPVKAPED

SEQ ID NO: 30 - protein, 406 aa, Escherichia coli
MKRAVITGLGIVSSIGNNQQEVLASLREGRSGITFSQELKDSGMRSHVWGNVKLDTTGLIDRKVVRFMSDASIYAFLSMEQAIADAGLSPEAYQNNPRVGLIAGSGGGSPRFQVFGADAMRGPRGLKAVGPYVVTKAMASGVSACLATPFKIHGVNYSISSACATSAHCIGNAVEQIQLGKQDIVFAGGGEELCWEMACEFDAMGALSTKYNDTPEKASRTYDAHRDGFVIAGGGGMVVVEELEHALARGAHIYAEIVGYGATSDGADMVAPSGEGAVRCMKMAMHGVDTPIDYLNSHGTSTPVGDVKELAAIREVFGDKSPAISATKAMTGHSLGAAGVQEAIYSLLMLEHGFIAPSINIEELDEQAAGLNIVTETTDRELTTVMSNSFGFGGTNATLVMRKLKD

SEQ ID NO: 31 - protein, 239 aa, Escherichia coli
MVIKAQSPAGFAEEYIIESIWNNRFPPGTILPAERELSELIGVTRTTLREVLQRLARDGWLTIQHGKPTKVNNFWETSGLNILETLARLDHESVPQLIDNLLSVRTNISTIFIRTAFRQHPDKAQEVLATANEVADHADAFAELDYNIFRGLAFASGNPIYGLILNGMKGLYTRIGRHYFANPEARSLALGFYHKLSALCSEGAHDQVYETVRRYGHESGEIWHRMQKNLPGDLAIQGR

SEQ ID NO: 32 - protein, 560 aa, Pseudomonas putida
MLQTRIIKPAEGAYAYPLLIKRLLMSGSRYEKTREIVYRDQMRLTYPQLNERIARLANVLTEAGVKAGDTVAVMDWDSHRYLECMFAIPMIGAVVHTINVRLSPEQILYTMNHAEDRVVLVNSDFVGLYQAIAGQLTTVDKTLLLTDGPDKTAELPGLVGEYEQLLAAASPRYDFPDFDENSVATTFYTTGTTGNPKGVYFSHRQLVLHTLAEASVTGSIDSVRLLGSNDVYMPITPMFHVHAWGIPYAATMLGMKQVYPGRYEPDMLVKLWREEKVTFSHCVPTILQMLLNCPNAQGQDFGGWKIIIGGSSLNRSLYQAALARGIQLTAAYGMSETCPLISAAHLNDELQAGSEDERVTYRIKAGVPVPLVEAAIVDGEGNFLPADGETQGELVLRAPWLTMGYFKEPEKSEELWQGGWLHTGDVATLDGMGYIDIRDRIKDVIKTGGEWVSSLDLEDLISRHPAVREVAVVGVADPQWGERPFALLVARDGHDIDAKALKEHLKPFVEQGHINKWAIPSQIALVTEIPKTSVGKLDKKRIRQDIVQWQASNSAFLSTL

SEQ ID NO: 33 - protein, 415 aa, Cuphea hookeriana
MVAAAASSAFFPVPAPGASPKPGKFGNWPSSLSPSFKPKSIPNGGFQVKANDSAHPKANGSAVSLKSGSLNTQEDTSSSPPPRTFLHQLPDWSRLLTAITTVFVKSKRPDMHDRKSKRPDMLVDSFGLESTVQDGLVFRQSFSIRSYEIGTDRTASIETLMNHLQETSLNHCKSTGILLDGFGRTLEMCKRDLIWVVIKMQIKVNRYPAWGDTVEINTRFSRLGKIGMGRDWLISDCNTGEILVRATSAYAMMNQKTRRLSKLPYEVHQEIVPLFVDSPVIEDSDLKVHKFKVKTGDSIQKGLTPGWNDLDVNQHVSNVKYIGWILESMPTEVLETQELCSLALEYRRECGRDSVLESVTAMDPSKVGVRSQYQHLLRLEDGTAIVNGATEWRPKNAGANGAISTGKTSNGNSVS

SEQ ID NO: 34 - protein, 340 aa, Acinetobacter sp.
MTTNVIHAYAAMQAGEALVPYSFDAGELQPHQVEVKVEYCGLCHSDVSVLNNEWHSSVYPVVAGHEVIGTITQLGSEAKGLKIGQRVGIGWTAESCQACDQCISGQQVLCTGENTATIIGHAGGFADKVRAGWQWVIPLPDELDPTSAGPLLCGGITVFDPILKHQIQAIHHVAVIGIGGLGHMAIKLLKAWGCEITAFSSNPNKTDELKAMGADHVVNSRDDAEIKSQQGKFDLLLSTVNVPLNWNAYLNTLAPNGTFHFLGVVMEPIPVPVGALLGGAKSLTASPTGSPAALRKLLEFAARKNIAPQIEMYPMSELNEAIERLHSGQARYRIVLKADF

SEQ ID NO: 35 - protein, 525 aa, Saccharomyces sp.
MNEIDEKNQAPVHQECLKEMIQNGHARRMGSVEDLYVALNRQNLYRNFCTYGELSDYCTRDQLTLALREICLKNPTLLHIVLPTRWPNHENYYRSSEYYSRPHPVHDYISVLQELKLSGVVLNEQPEYSAVMKQILEEFKNSKGSYTAKIFKLTTTLTIPYFGPTGPSWRLICLPEEHTEKWKKFIFVSNHCMSDGRSSIHFFHDLRDELNNIKTPPKKLDYIFKYEEDYQLLRKLPEPIEKVIDFRPPYLFIPKSLLSGFIYNHLRFSSKGVCMRMDDVEKTDDVVTEIINISPTEFQAIKANIKSNIQGKCTITPFLHVCWFVSLHKWGKFFKPLNFEWLTDIFIPADCRSQLPDDDEMRQMYRYGANVGFIDFTPWISEFDMNDNKENFWPLIEHYHEVISEALRNKKHLHGLGFNIQGFVQKYVNIDKVMCDRAIGKRRGGTLLSNVGLFNQLEEPDAKYSICDLAFGQFQGSWHQAFSLGVCSTNVKGMNIVVASTKNVVGSQESLEELCSIYKALLLGP

SEQ ID NO: 36 - protein, 295 aa, Acinetobacter sp.
MNKKLEALFRENVKGKVALITGASSGIGLTIAKRIAAAGAHVLLVARTQETLEEVKAAIEQQGGQASIFPCDLTDMNAIDQLSQQIMASVDHVDFLINNAGRSIRRAVHESFDRFHDFERTMQLNYFGAVRLVLNLLPHMIKRKNGQIINISSIGVLANATRFSAYVASKAALDAFSRCLSAEVLKHKISITSIYMPLVRTPMIAPTKIYKYVPTLSPEEAADLIVYAIVKRPKRIATHLGRLASITYAIAPDINNILMSIGFNLFPSSTAALGEQEKLNLLQRAYARLFPGEHW

SEQ ID NO: 37 - protein, 661 aa, Marinobacter sp.
MNYFLTGGTGFIGRFLVEKLLARGGTVYVLVREQSQDKLERLRERWGADDKQVKAVIGDLTSKNLGIDAKTLKSLKGNIDHVFHLAAVYDMGADEEAQAATNIEGTRAAVQAAEAMGAKHFHHVSSIAAAGLFKGIFREDMFEEAEKLDHPYLRTKHESEKVVREECKVPFRIYRPGMVIGHSETGEMDKVDGPYYFFKMIQKIRHALPQWVPTIGIEGGRLNIVPVDFVVDALDHIAHLEGEDGNCFHLVDSDPYKVGEILNIFCEAGHAPRMGMRIDSRMFGFIPPFIRQSIKNLPPVKRITGALLDDMGIPPSVMSFINYPTRFDTRELERVLKGTDIEVPRLPSYAPVIWDYWERNLDPDLFKDRTLKGTVEGKVCVVTGATSGIGLATAEKLAEAGAILVIGARTKETLDEVAASLEAKGGNVHAYQCDFSDMDDCDRFVKTVLDNHGHVDVLVNNAGRSIRRSLALSFDRFHDFERTMQLNYFGSVRLIMGFAPAMLERRRGHVVNISSIGVLTNAPRFSAYVSSKSALDAFSRCAAAEWSDRNVTFTTINMPLVKTPMIAPTKIYDSVPTLTPDEAAQMVADAIVYRPKRIATRLGVFAQVLHALAPKMGEIIMNTGYRMFPDSPAAAGSKSGEKPKVSTEQVAFAAIMRGIYW

SEQ ID NO: 38 - protein, 246 aa, Cupriavidus necator
MTQRIAYVTGGMGGIGTAICQRLAKDGFRVVAGCGPNSPRREKWLEQQKALGFDFIASEGNVADWDSTKTAFDKVKSEVGEVDVLINNAGITRDVVFRKMTRADWDAVIDTNLTSLFNVTKQVIDGMADRGWGRIVNISSVNGQKGQFGQTNYSTAKAGLHGFTMALAQEVATKGVTVNTVSPGYIATDMVKAIRQDVLDKIVATIPVKRLGLPEEIASICAWLSSEESGFSTGADFSLNGGLHMG

SEQ ID NO: 39 - protein, 284 aa, Cupriavidus necator
MSIRTVGIVGAGTMGNGIAQACAVVGLNVVMVDISDAAVQKGVATVAGSLDRLIKKEKLTEADKASALARIKGSTSYDDLKATDIVIEAATENYDLKVKILKQIDAIVGENVIIASNTSSISITKLAAVTSRADRFIGMHFFNPVPVMALVELIRGLQTSDITHAAVEALSKQLGKYPITVRNSPGFVVNRILCPMINEAFCVLGEGLASPEEIDEGMKLGCNHPIGPLALADMIGLDTMLAVMEVLYTEFADPKYRPAMLMREMVAAGYLGRKTGRGVYVYSK

SEQ ID NO: 40 - protein, 284 aa, Cupriavidus necator
MSIRTVGIVGAGTMGNGIAQACAVVGLNVVMVDISDAAVQKGVATVASSLDRLIKKEKLTEADKASALARIKGSTSYDDLKATDIVIEAATENYDLKVKILKQIDGIVGENVIIASNTSSISITKLAAVTSRADRFIGMHFFNPVPVMALVELIRGLQTSDTTHAAVEALSKQLGKYPITVKNSPGFVVNRILCPMINEAFCVLGEGLASPEEIDEGMKLGCNHPIGPLALADMIGLDTMLAVMEVLYTEFADPKYRPAMLMREMVAAGYLGRKTGRGVYVYSK

SEQ ID NO: 41 - protein, 246 aa, Cupriavidus taiwanensis
MKLQGRVAIITGAAAGIGFATAQRFAEDGAIVVLCDVQEARVREAAARLAATGATVSAYRVDVTRRDEVDAMVAAVLAAHQRVDILVNNAGITKDARLAKMTEAQFDAVIDVNLKGVFNCAQAVAGLMTEQGKGVILNASSVVGLYGNFGQTNYAASKFGVIGFTKTWARELGPKGVRVNAVCPGFVNTEILQTVPDKVLDGMTSSCWLRRLAEPAEIASIYAFLASDDASYVNGVAIEASGGMSL

SEQ ID NO: 42 - protein, 243 aa, Bacillus sp.
  source 1..243: note = strain SG-1
MSRLQDKVAIITGAANGIGLEAARVFMKEGAKVVIADFNEAAGKEAVEANPGVVFIRVDVSDRESVHRLVENVAERFGKIDILINNAGITRDSMLSKMTVDQFQQVINVNLTGVFHCTQAVLPYMAEQGKGKIINTSSVTGTYGNVGQTNYAAAKAGVIGMTKTWAKELARKGINVNAVAPGFTETAMVAEVPEKVIEKMKAQVPMGRLGKPEDIANAYLFLASHESDYVNGHVLHVDGGIMM

SEQ ID NO: 43 - protein, 246 aa, Burkholderia multivorans
MSQRIAYVTGGMGGIGTSICQRLSKDGFKVVAGCGPNSPRRVKWLEEQKALGFDFIASEGNVGDWDSTKAAFDKVKAEVGEVDVLVNNAGITRDVVFRKMTHEDWTAVIDTNLTSLFNVTKQVIDGMVERGWGRIINISSVNGQKGQFGQTNYSTAKAGIHGFTMALAQEVATKGVTVNTVSPGYIGTDMVKAIRPDVLEKIVATIPVRRLGTPEEIGSIVAWLASNDSGFATGADFSLNGGLHMG

SEQ ID NO: 44 - protein, 246 aa, Pseudomonas putida
MTKRIAYVTGGMGGIGTSICQRLHKDGYTVVAGCGPNSPRRVKWLEEQKANGYDFIASEGNVGDWESTKNAFDKVKAEVGEVDILVNNAGITRDVVFRKMTHEDWTAVIDTNLTSLFNVTKQVVDGMVERGFGRIINISSVNGQKGQFGQTNYSTAKAGIHGFTMALAQEVATKGVTVNTVSPGYIGTDMVKAIRPEVLEKIVATIPVRRLGRPDEIGSIVSWLASEESGFSTGADFSLNGGLHMG

SEQ ID NO: 45 - protein, 284 aa, Megasphaera elsdenii
MSKVMVIGAGTMGSGIAQVFATHGWDVVLNDIKDEFIAGGKAKIEKQLTKQVSKGKMTQEDKDATMARISGFVTRTPDNMKDIDLVVEAAIEDLKIKSAIFKELDEFCPEHTILASNTSSLAITQIGAATKRPDKVIGMHFFNPAPVMKLIEVINGIATSEETYDKIDKLSKDLGKTPVKVNDFPGFAGNRIVIPMLNEAIQALMEGVASKEDIDNVCKLGFNHPMGPLALCDLIGNDVVLHVMEVLYDGFGDPKYRPCALLKKYVQAGYLGRKTGKGFYDYTK

(19) United States
(12) Patent Application Publication
(10) Pub. No.: US 2025/0207159 A1
(43) Pub. Date: Jun. 26, 2025

(54) Microorganisms And Methods For Production Of Fatty Acid Derivatives With Reduced Levels Of Byproducts

(71) Applicant: Genomatica, Inc., San Diego, CA (US)

(72) Inventor: Andreas W. SCHIRMER, San Diego, CA (US)

(21) Appl. No.: 18/846,710

(22) PCT Filed: Mar. 15, 2023
(86) PCT No.: PCT/US2023/064485

§ 371 Date: Sep. 13, 2024

Related U.S. Application Data
(60) Provisional application No. 63/320,581, filed on Mar. 16, 2022.

(51) Int. Cl. C12P 7/6436; C07C 57/03; C12N 1/20; C12N 9/00; C12N 9/16; C12N 9/88

(52) U.S. Cl. CPC C12P 7/6436; C07C 57/03; C12N 1/20; C12N 9/16; C12N 9/88; C12N 9/93; C12Y 301/02014; C12Y 402/01119; C12Y 402/01134; C12Y 602/01003

(57) ABSTRACT

Recombinant microbes comprising a novel pathway for producing fatty acid derivatives, such as fatty acid methyl esters, fatty acid ethyl esters, fatty alcohols, and fatty alcohol acetates, while generating low levels of 3-hydroxy fatty acid byproducts are provided herein. The addition of a heterologous R-3-hydroxy-acyl-CoA dehydratase or a heterologous R-specific enoyl-CoA hydratase and optionally a heterologous trans-2-enoyl-CoA reductase to a recombinant microbe reduces the amount of 3-hydroxy fatty acid byproducts produced by the acyl-ACP dependent fatty acid biosynthesis pathway of the recombinant microbe. Methods of producing the fatty acid derivatives are also provided, in addition to cell cultures and fatty acid derivative compositions produced by the recombinant microbes. The fatty acid derivatives produced by the recombinant microbes may be used to produce fragrances, pheromones, pharmaceutical agents, nutraceuticals or precursors thereof.

### REFERENCE TO AN ELECTRONIC SEQUENCE LISTING

This application contains references to amino acid sequences and/or nucleic acid sequences which have been submitted concurrently herewith as the sequence listing xml file entitled “ST26_SL_13_Mar_2023.xml”, file size 53 KiloBytes (KB), created on 13 Mar. 2023. The aforementioned sequence listing is hereby incorporated by reference in its entirety.

### FIELD

The disclosure relates to the field of specialty chemicals and methods for their preparation. The disclosure provides novel biosynthetic pathways, recombinant cells or microbes, and methods, for the production of fatty acids and derivatives thereof, or compositions containing fatty acids and derivatives thereof, with a reduced amount of unwanted byproducts. The unwanted byproducts generally are derived from or generated from one or more intermediates of an acyl-ACP dependent fatty acid biosynthetic pathway. The unwanted byproducts can be, for example, fatty acids or derivatives thereof containing a 3-hydroxy (3-OH) group, or fatty acids or derivatives thereof containing a 3-oxo (or beta-keto) group, or a combination thereof. For example, the disclosure provides recombinant cells or microbes or microorganisms that comprise, or that are engineered or modified to express i) one of a heterologous R-3-hydroxy-acyl-CoA dehydratase or a heterologous R-specific enoyl-CoA hydratase, and optionally, ii) a heterologous trans-2-enoyl-CoA reductase. In some embodiments, the recombinant cells or microbes or microorganisms comprise, or are engineered or modified to additionally express iii) a 3-oxoacyl-CoA reductase, a β-ketoacyl-CoA-reductase, or a 3-hydroxy acyl-CoA dehydrogenase. The disclosure further provides methods for the production of various fatty acid derivatives and compositions comprising the same, by culturing the recombinant cells, microbes or microorganisms provided herein.

### BACKGROUND

The use of biosynthetic pathways for the sustainable production of chemicals and fuels holds great promise for addressing the challenges of fuel and chemical production without contributing to greenhouse gas emissions. Unfortunately, byproducts produced by biosynthetic pathways during production of a target compound can reduce the yield or titer of the target compound, and the efficiency of the production method. Furthermore, byproducts can complicate the downstream recovery and purification of a target compound, and, in certain cases, can be toxic to the producing cells, which can further reduce the yield, titer, and/or productivity of the target product (or the product of interest). It is therefore desirable to avoid (e.g., eliminate) or reduce the production of unwanted byproducts. The novel biosynthetic pathways, recombinant microorganisms, and methods described herein, are designed to convert unwanted byproducts or intermediates to target compounds.

The microbial production of fatty acid derivatives, such as, for example, fatty esters, fatty alcohols, or fatty alcohol acetates, can be achieved by acyl-ACP dependent or acyl-ACP independent biosynthetic pathways. The method of reducing unwanted byproducts described herein includes a combination of an acyl-ACP-dependent fatty acid biosynthetic pathway, together with parts of an acyl-CoA dependent fatty acid biosynthetic pathway.

In most bacteria, fatty acid species are produced by type II fatty acid biosynthesis. Fatty acid biosynthesis is a biosynthetic process by which fatty acids (and derivatives thereof) are produced from acetyl-CoA through the action of enzymes known as fatty acid synthases (sometimes referred to herein as fatty acid biosynthetic or biosynthesis enzymes, or fatty acid biosynthetic/biosynthesis pathway enzymes). Acetyl-CoA, which is typically generated from carbohydrates via the glycolytic pathway, is first converted to malonyl-CoA by the action of acetyl-CoA carboxylase (AccABCD), and the malonyl-CoA is converted to malonyl-ACP by the action of malonyl-CoA:ACP transacylase (also known as [acyl-carrier-protein] S-malonyltransferase, or FabD; EC 2.3.1.39). The fatty acid biosynthetic cycle is initiated by the condensation of malonyl-ACP and acetyl-CoA (by 3-oxoacyl-[ACP] synthase III, also known as β-ketoacyl-ACP synthase III or FabH; EC 2.3.1.180) to form acetoacetyl-ACP. The elongation cycle, or the fatty acid biosynthesis reductive cycle, then begins with the condensation of acetoacetyl-ACP with an acyl-ACP, by β-ketoacyl-ACP synthase I (e.g., FabB) and β-ketoacyl-ACP synthase II (e.g., FabF), to produce a β-keto-acyl-ACP. The β-keto-acyl-ACP is reduced by β-ketoacyl-ACP reductase (e.g., FabG) to produce a β-hydroxy-acyl-ACP (or a 3-hydroxy-acyl-ACP), which is then dehydrated to a trans-2-enoyl-ACP by a β-hydroxyacyl-ACP dehydratase (e.g., FabA or FabZ). The final step in each cycle is catalyzed by enoyl-ACP reductase (e.g., FabI) that converts trans-2-enoyl-ACP to acyl-ACP. FabA can also isomerize trans-2-enoyl-ACP to cis-3-enoyl-ACP, which can bypass FabI and can be used by FabB (typically for up to an aliphatic chain length of C16) to produce β-keto-acyl-ACP.

The fatty acyl-ACP product of the fatty acid biosynthesis reductive cycle can be converted to a fatty acid by an acyl-ACP thioesterase, which can then be converted to a variety of different fatty acid derivatives by the appropriate fatty acid derivative enzyme(s). For example, the fatty acid can be converted to a fatty aldehyde by the action of a carboxylic acid reductase (CAR), or to a fatty alcohol by a CAR and an alcohol dehydrogenase (ADH). The fatty acid can additionally be converted to an acyl-CoA by an acyl-CoA synthetase, and the acyl-CoA can be converted to a fatty acid ester by an ester synthase; to a fatty aldehyde by an acyl-CoA reductase; to a fatty alcohol by an acyl-CoA reductase and an alcohol dehydrogenase or by a fatty alcohol forming acyl-CoA reductase; or to a fatty alcohol acetate by an acyl-CoA reductase, an alcohol dehydrogenase, and an alcohol acetyl-CoA transferase (also known as alcohol-O-acetyl-transferase) (see, e.g., FIG. 1). Other fatty acid derivatives, such as triols, tetrols, α,ω-diols, α,ω-diacids, α,ω-diesters, ω-hydroxy fatty acids, w-hydroxy fatty esters, fatty amines, fatty amides, alkanes, alkenes, terminal and internal olefins, and others, can also be made from the fatty acids, or from the acyl-ACP or acyl-CoA precursors, by methods known in the art and/or described elsewhere herein. Such fatty acid derivatives, and the biosynthetic pathways for making them, are described, for example, in WO 2014/201474, WO 2013/154721, WO 2015/085271, WO 2016/011430, WO 2019/217226, and WO 2022/212119, each of which is incorporated by reference in its entirety herein.

Yeast and other eukaryotes have a type I fatty acid biosynthesis that uses a fatty acid synthase complex, where most of these enzyme activities are fused in one big protein including ACP, and fatty acids are off-loaded as fatty acyl-CoAs.

FIG. 1 depicts an example of an acyl-ACP dependent biosynthetic pathway for the production of fatty esters, fatty alcohols, or fatty alcohol acetates. As shown in FIG. 1, the 3-hydroxy-acyl-ACP intermediate also can be converted to a 3-hydroxy fatty acid (3-OH FA) by the action of an acyl-ACP thioesterase, or a thioesterase with preference for acyl-ACPs (EC 3.1.2.14, EC 3.1.2.21). The 3-OH FA can be converted to a variety of different fatty acid derivatives containing a 3-hydroxy group by the action of the appropriate fatty acid derivative enzyme(s). For example, the 3-OH FA can be converted to a 3-OH fatty aldehyde by a carboxylic acid reductase (CAR), which can then be converted to, for example, a 1,3-fatty diol by an alcohol dehydrogenase, or to a 3-hydroxy fatty amine by a transaminase or aminotransferase. The 3-OH FA can also be converted (or activated) to a 3-OH-acyl-CoA by the action of an acyl-CoA synthetase (also known as acyl-CoA ligase, EC 6.2.1.3), and the 3-OH-acyl-CoA can be converted to a variety of different fatty acid derivatives containing a 3-hydroxy group by the action of the appropriate fatty acid derivative enzyme(s). For example, the 3-OH-acyl-CoA can be converted to a 3-OH fatty acid ester by an ester synthase with a preference for acyl-CoAs (e.g., EC 2.3.1.20); or to a 3-OH fatty aldehyde by an acyl-CoA-reductase (e.g., EC 1.2.1.50, or EC 1.2.1.84); or to a 1,3-fatty diol by an acyl-CoA-reductase and an alcohol dehydrogenase (e.g., EC 1.1.1.1); or to a fatty alcohol 1,3-diactetate by an acyl-CoA-reductase, an alcohol dehydrogenase, and an alcohol acetyl-CoA transferase (e.g., EC 2.3.1.84) (see, e.g., FIG. 1).

The reductive cycle of fatty acid biosynthesis can be manipulated to direct synthesis towards particular products. The “driving force” of the reductive cycle is the last step (i.e., the formation of acyl-thioesters), carried out by trans-2-enoyl thioester reductase (see e.g., Heath and Rock, J. Biol. Chem, (1995) 270:26538-26542). The reversible enzymatic reactions leading to the formation of enoyl-thioester (e.g., enoyl-ACP and enoyl-CoA) intermediates are catalyzed by 3-hydroxy-acyl-thioester dehydratases and/or enoyl-thioester hydratases. Formation of the 3-hydroxy-acyl-CoA intermediate is thermodynamically favored, which leads to the generation of 3-hydroxy fatty acid derivatives (e.g., 3-hydroxy fatty acid methyl and ethyl esters, 1,3-fatty diols, fatty alcohol 1,3-diacetates and others; see, e.g., FIG. 1), which can be undesirable for certain uses and applications. Thus, there remains a need to efficiently produce fatty acids and derivatives thereof that do not contain a 3-hydroxy group (such as, e.g., fatty acid methyl esters or fatty acid ethyl esters), while reducing (or eliminating, or substantially eliminating) the production of byproducts, such as 3-hydroxy fatty acids and derivatives thereof.

As described herein, the 3-OH group containing fatty acids and derivatives thereof can be undersirable in certain cases and for certain applications, and thus, these 3-OH fatty acids and 3-OH fatty acid derivatives can be considered as byproducts. If the substrate specificity (or selectivity) of the thioesterase for 3-hydroxy acyl-ACPs is high, the formation of unwanted 3-OH group containing fatty acids and derivatives thereof (e.g., 3-hydroxy fatty esters, 1,3 fatty diols or fatty alcohol 1,3-diacetates) can significantly impact the yield of the bioprocess and the downstream recovery and purification of the target fatty acid and fatty acid derivative products which do not contain a 3-OH group (e.g., fatty esters, fatty alcohols, or fatty alcohol acetates).

To address this problem, provided herein is a “bypass mechanism” or “bypass pathway” that efficiently converts unwanted 3-hydroxy fatty acids, via acyl-CoA or CoA-containing intermediates, to the target products that do not contain a 3-OH group, e.g. fatty esters, fatty alcohols, or fatty alcohol acetates, thereby reducing or eliminating or substantially eliminating the unwanted 3-OH byproducts (e.g., 3-hydroxy fatty esters, 1,3 fatty diols, or fatty alcohol 1,3-diacetates). For example, as described herein, and as shown, for example, in FIG. 2, 3-hydroxy fatty acids are activated to the corresponding 3-hydroxy-acyl-CoAs by acyl-CoA synthetase (also known as acyl-CoA ligase), which can already be part of the biochemical pathway towards the target products. The 3-hydroxy-acyl-CoAs (3-OH-acyl-CoAs) can be converted to trans-2-enoyl-CoAs by an R-3-hydroxy-acyl-CoA dehydratase (e.g., EC 4.2.1.134, or EC 4.2.1.55) or by an R-specific enoyl-CoA hydratase (e.g., EC 4.2.1.119, EC 4.2.1.17), and the trans-2-enoyl-CoAs can be converted to acyl-CoAs by a trans-2-enoyl-CoA reductase (e.g., EC 1.3.1.44). The acyl-CoAs then enter the biochemical or biosynthetic pathways towards the target products, e.g. fatty esters, fatty alcohols, or fatty alcohol acetates (see FIG. 2). This pathway can reduce, eliminate, or substantially eliminate, the amount (e.g., in terms of yield, titer, weight %, and/or productivity) of 3-OH group containing fatty acids and derivatives thereof.

In addition to 3-OH group containing byproducts, derived from 3-OH-acyl-ACP intermediates, the acyl-ACP dependent biosynthetic pathway can result in unwanted or undersireable fatty acid or fatty acid derivative byproducts containing a 3-oxo (or beta-keto) group. Such 3-oxo group containing byproducts, which have a ketone functional group at position 3 of the carbon chain (where the carboxyl group carbon is assigned position number 1), and can include, for example, 3-oxo fatty acids, 3-oxo fatty acid esters, 3-oxo fatty alcohols, and 3-oxo fatty alcohol acetates (3-oxo fatty alcohol acetate esters). The 3-oxo group containing byproducts are generated from or derived from 3-keto-acyl-ACP (or beta (β)-keto-acyl-ACP) intermediates of the acyl-ACP dependent biosynthetic pathway (see, e.g., FIG. 3).

For example, as shown in FIG. 3, 3-keto-acyl-ACP (also called β-keto-acyl-ACP) intermediates of the acyl-ACP dependent fatty acid biosynthesis cycle can be hydrolyzed to 3-oxo fatty acids, by thioesterases, such as, for example, native/endogenous (or heterologous) promiscuous acyl-ACP thioesterases. The 3-oxo fatty acids can be converted by an (endogenous/native or heterologous) acyl-CoA synthetase to the corresponding 3-oxo-acyl-CoAs. As with the 3-OH fatty acids and 3-OH-acyl-CoAs, the 3-oxo fatty acids and 3-oxo-acyl-CoAs can be converted into various different 3-oxo group containing fatty acid derivative byproducts by one or more native or heterologous fatty acid derivative enzymes. For example, the 3-oxo fatty acids can be converted to 3-oxo fatty aldehydes by a CAR, which can then be converted to, for example, a 3-oxo fatty alcohol by an alcohol dehydrogenase, or to a 3-oxo fatty amine by a transaminase or aminotransferase. The 3-oxo fatty acids could also be converted to 3-hydroxy fatty acids and derivatives thereof by an enzyme capable of reducing the 3-oxo group to a 3-hydroxy group. The 3-oxo-acyl-CoAs can be converted to 3-oxo fatty acid esters by an ester synthase; or to 3-oxo fatty aldehydes by an acyl-CoA-reductase; or to 3-oxo fatty alcohols by an acyl-CoA-reductase and an alcohol dehydrogenase, or by a fatty-alcohol forming acyl-CoA reductase; or to 3-oxo fatty alcohol actetate esters by an acyl-CoA-reductase, an alcohol dehydrogenase, and an alcohol acetyl-CoA transferase, or by a fatty-alcohol forming acyl-CoA reductase and an alcohol acetyl-CoA transferase.

As described herein, the 3-oxo group containing fatty acids and derivatives thereof can be undersirable in certain cases and for certain applications, and thus, these 3-oxo fatty acids and 3-oxo fatty acid derivatives can also be considered as byproducts. If the substrate specificity (or selectivity) of the thioesterase for 3-keto-acyl-ACPs is high, the formation of unwanted 3-oxo group containing fatty acids and derivatives thereof can significantly impact the yield of the bioprocess and the downstream recovery and purification of the target fatty acid and fatty acid derivative products which do not contain a 3-oxo group (e.g., fatty esters, fatty alcohols, or fatty alcohol acetates). Thus, to address this problem, also provided herein is a “bypass mechanism” or “bypass pathway” that efficiently converts unwanted 3-oxo fatty acids, via acyl-CoA or CoA-containing intermediates, to the target products that do not contain a 3-oxo group (e.g. fatty esters, fatty alcohols, or fatty alcohol acetates), thereby reducing or eliminating or substantially eliminating the unwanted 3-oxo byproducts (e.g., 3-oxo fatty acids, 3-oxo fatty esters, 3-oxo fatty alcohols, or 3-oxo fatty alcohol acetates).

For example, as described herein, and as shown, for example, in FIG. 3, 3-oxo fatty acids are activated to the corresponding 3-oxo-acyl-CoAs (also known as beta- or β-keto-acyl-CoAs) by acyl-CoA synthetase (also known as acyl-CoA ligase), which can already be part of the biochemical pathway towards the target products. The 3-oxo-acyl-CoAs can then be converted (reduced) to the corresponding 3-hydroxy-acyl-CoAs by a 3-oxoacyl-CoA reductase, β-ketoacyl-CoA-reductase, or 3-hydroxy acyl-CoA dehydrogenase (e.g., having EC 1.1.1.35, EC 1.1.1.36, or EC 1.1.1.157). As described above, the 3-hydroxy-acyl-CoAs (3-OH-acyl-CoAs) can be converted to trans-2-enoyl-CoAs by an R-3-hydroxy-acyl-CoA dehydratase (e.g., EC 4.2.1.134, or EC 4.2.1.55) or by an R-specific enoyl-CoA hydratase (e.g., EC 4.2.1.119, EC 4.2.1.17), and the trans-2-enoyl-CoAs can be converted to acyl-CoAs by a trans-2-enoyl-CoA reductase (e.g., EC 1.3.1.44). The acyl-CoAs then enter the biochemical or biosynthetic pathways towards the target products, e.g. fatty esters, fatty alcohols, or fatty alcohol acetates (see FIG. 3). This pathway can reduce, eliminate, or substantially eliminate, the amount (e.g., in terms of yield, titer, weight %, and/or productivity) of 3-oxo group containing fatty acids and derivatives thereof. Although R-3-hydroxy acyl-CoA dehydratase, R-specific enoyl-CoA hydratase, or trans-2-enoyl-CoA reductase can be part of certain acyl-ACP independent fatty acid biosynthetic pathways (see, e.g., Dellomonaco et al., Nature, 476:355-9 (2011); and WO 2015/010103) and fatty acid elongation pathways in eukaryotes (see, e.g., Dittrich et al., Eur. J. Biochem. 252, 477-485 (1998)), these enzyme activities have not been combined with an acyl-ACP dependent fatty acid biosynthesis pathway with the purpose of reducing or eliminating byproduct formation.

The “bypass” pathways and methods provided herein can be employed to reduce byproducts in any biochemical pathways that include acyl-CoA intermediates, as well as 3-hydroxy fatty acid and/or 3-oxo fatty acid intermediates. Additional examples are biochemical pathways towards fatty aldehydes (reduces 3-hydroxy- and/or 3-oxo-fatty aldehydes), fatty amines (reduces 3-hydroxy- and/or 3-oxo-fatty amines), ω-hydroxy fatty esters (reduces ω-hydroxy fatty esters with a 3-hydroxy or 3-oxo group), ω-carboxy fatty esters (reduces ω-carboxy fatty esters with a 3-hydroxy or 3-oxo group), α/ω-fatty diesters esters (reduces α/ω-fatty diesters esters with a 3-hydroxy or 3-oxo group), etc.

### SUMMARY

Provided herein are novel biosynthetic pathways, methods, and recombinant cells or microbes, for the production of fatty acids and derivatives thereof, or compositions comprising fatty acids and derivatives thereof, with a reduced amount (e.g., titer, yield, concentration, or weight %) of unwanted byproducts. The unwanted byproducts generally are derived from or generated from one or more intermediates of an acyl-ACP dependent fatty acid biosynthetic pathway. Such unwanted byproducts include fatty acids and derivatives thereof that contain a 3-hydroxy (3-OH) group and/or fatty acids and derivatives thereof that contain a 3-oxo (or beta-keto) group. The recombinant cells or microbes comprise or express an acyl-ACP dependent fatty acid biosynthetic pathway. Typically, the acyl-ACP dependent fatty acid biosynthetic pathway is native and/or endogenous, but the pathway can include heterologous enzymes and/or native enzymes encoded by exogenous nucleic acid sequences. As described herein, the acyl-ACP dependent fatty acid biosynthetic pathway results in the production of 3-hydroxy-acyl-ACP and/or 3-keto-acyl-ACP (also referred to as 3-oxo-acyl-ACP or beta-keto-acyl-ACP) intermediates, which can be converted to 3-hydroxy (3-OH) free fatty acids and 3-oxo fatty acids, respectively, by thioesterases, such as native or endogenous promiscuous thioesterases that can utilize 3-OH-acyl-ACP and/or 3-keto-acyl-ACP as a substrate. The 3-OH fatty acids and/or 3-oxo fatty acids can then be directly converted to various different 3-OH fatty acid derivatives and 3-oxo fatty acid derivatives, respectively, by native or heterologous fatty acid derivative enzymes, or the 3-OH fatty acids and/or 3-oxo fatty acids can be converted to 3-OH-acyl-CoA and 3-oxo-acyl-CoA intermediates first by an acyl-CoA synthetase (which can be native or heterologous), and the 3-OH-acyl-CoAs and/or 3-oxo-acyl-CoAs can be converted to 3-OH fatty acid derivatives and 3-oxo fatty acid derivatives, respectively.

This disclosure provides novel biosynthetic pathways (for example, see, FIG. 2 and FIG. 3), also referred to herein as “bypass pathways”, that utilize or include A) an acyl-ACP thioesterase and an acyl-CoA synthetase; B) one of a heterologous R-3-hydroxy acyl-CoA dehydratase or a heterologous R-specific enoyl-CoA hydratase; and C) optionally, a heterologous trans-2-enoyl-CoA reductase. The novel biosynthetic or bypass pathways can further include D) one of a heterologous 3-oxoacyl-CoA reductase, a heterologous β-ketoacyl-CoA-reductase, or a heterologous 3-hydroxy acyl-CoA dehydrogenase. These novel pathways preferentially produce fatty acids and fatty acid derivatives, such as, but not limited to, for example, fatty acid alkyl esters, fatty alcohols, and fatty alcohol acetates, while reducing, or eliminating, or substantially eliminating, the production of 3-hydroxy group containing and/or 3-oxo group containing byproducts, such as, for example, 3-hydroxy fatty acids, 3-hydroxy fatty aldehydes, 3-hydroxy fatty amines, 3-hydroxy fatty amides, 3-hydroxy fatty acid alkyl esters, 1,3-fatty diols, and fatty alcohol 1,3-diacetates, and/or 3-oxo fatty acids, 3-oxo fatty aldehydes, 3-oxo fatty amines, 3-oxo fatty amides, 3-oxo fatty acid alkyl esters, 3-oxo fatty alcohols, and 3-oxo fatty alcohol acetate esters. Other fatty acid derivatives and their corresponding 3-OH and 3-oxo containing versions, are described above and elsewehere herein, and/or are known or described by the art. For example, the pathways, methods, and recombinant microbes provided herein also can produce fatty acids and/or derivatives thereof, and compositions comprising fatty acids and/or derivatives thereof, with reduced amounts of ω-hydroxy fatty acid, ω-hydroxy fatty ester, ω-carboxy fatty ester, α/ω-fatty diester, α/ω-fatty diacid, α/ω-fatty diol, and other, byproducts containing a 3-hydroxy or 3-oxo functional group.

Thus, disclosed herein is a recombinant cell, microorganism, or microbe, comprising one or more of: A) an acyl-ACP thioesterase and an acyl-CoA synthetase; B) one of a heterologous-R-3-hydroxy acyl-CoA dehydratase or a heterologous R-specific enoyl-CoA hydratase; and optionally, C) a heterologous trans-2-enoyl-CoA reductase. Also provided herein is a recombinant cell, microorganism, or microbe, comprising one or more of: A) an acyl-ACP thioesterase and an acyl-CoA synthetase; B) one of a heterologous 3-oxoacyl-CoA reductase, a heterologous β-ketoacyl-CoA-reductase, or a heterologous 3-hydroxy acyl-CoA dehydrogenase; C) one of a heterologous R-3-hydroxy acyl-CoA dehydratase or a heterologous R-specific enoyl-CoA hydratase; and optionally, D) a heterologous trans-2-enoyl-CoA reductase. The acyl-ACP thioesterase and/or the acyl-CoA synthetase can be native to the recombinant cell, microbe, or microorganism (i.e., naturally made by the cell, microbe or microorganism), and can be expressed (e.g., by an endogenous encoding nucleic acid sequence) or can be overexpressed (e.g., by an exogenous nucleic acid sequence). Alternatively or additionally, the acyl-ACP thioesterase and/or the acyl-CoA synthetase can be heterologous, i.e., from a different species than the recombinant cell, micobe, or microorganism. In some embodiments, the enzyme or polypeptide with 3-oxoacyl-CoA reductase, β-ketoacyl-CoA-reductase, or 3-hydroxy acyl-CoA dehydrogenase activity can be native to the recombinant cell or microbe. For example, E. coli FabG, or homologs thereof from other species, can convert 3-oxo-acyl-CoA to 3-hydroxy-acyl-CoA. However, the activity of FabG on acyl-CoA intermediates, such as 3-oxo-acyl-CoA, can be inefficient/low.

The recombinant cell, microorganism, or microbe can further comprise one or more additional fatty acid biosynthesis or fatty acid derivative enzymes (or regulatory factors), including, but not limited to, for example, an ester synthase, a β-keto-acyl-ACP synthase (I, II, and/or III), an alcohol dehydrogenase, an alcohol-O-acetyl-transferase, an acyl-CoA reductase, a carboxylic acid reductase, an aldehyde reductase, a fatty alcohol forming acyl-CoA reductase, an omega-hydroxylase, a desaturase, a transaminase (or aminotransferase), an amine dehydrogenase, a decarbonylase, an aldehyde decarbonylase, an aldehyde oxidative deformylase, a decarboxylase, one or more subunits (e.g., AccA, AccB, AccC, and/or AccD) of an acetyl-CoA carboxylase (AccABCD), an OleA, an OleBCD, an OleABCD, an OleACD, an aldehyde dehydrogenase, an esterase or lipase, a CoA ligase/transferase, and/or FadR, any one of which can be expressed or overexpressed, and/or can be heterologous (i.e., from a different species) or native (i.e., from the same species) to the host cell, microorganism, or microbe, or a combination thereof. The heterologous enzyme or polypeptide is encoded by an exogenous nucleic acid sequence or gene, that can be expressed or overexpressed. The native enzyme or polypeptide can be encoded by an endogenous or exogenous gene or nucleic acid sequence, and can be expressed or overexpressed. Where a native enzyme or polypeptide is overexpressed, it is typically encoded by an exogenous gene or nucleic acid sequence. The native fatty acid biosynthesis or fatty acid derivative enzyme or polypeptide can be overexpressed, for example, by engineering the cell, microorganism or microbe to contain or express multiple copies of the encoding gene or nucleic acid, or by other techniques known in the art, such as by placing the encoding gene or nucleic acid under the control of a constitutive, inducible, or strong promoter, or by operably linking the encoding gene or nucleic acid sequence to another non-native regulatory element (e.g., 5′-UTR, ribosome binding site (RBS), or start codon, or a combination thereof). A variant of a native gene, for example, a variant with a non-native regulatory element or elements, can be considered as an exogenous gene, particularly where the whole gene, including the coding sequence and the regulatory elements, are introduced into the cell from outside the cell. The native fatty acid biosynthesis or fatty acid derivative enzyme or polypeptide can be exogenously expressed (or overexpressed), i.e., the nucleic acid sequence encoding the enzyme or polypeptide is introduced into the cell from the outside.

The recombinant cell, microorganism, or microbe may be a recombinant bacterium (e.g., a gamma-(γ)-proteobacterium, an alpha-(α)-proteobacterium, or a cyanobacterium), a recombinant yeast, or a recombinant algae. The recombinant cell, microorganism, or microbe can produce, or is capable of producing, one or more fatty acids and/or fatty acid derivatives, or a composition comprising one or more fatty acids and/or derivatives thereof, including, but not limited to, for example, free fatty acids, fatty esters, fatty alcohols, fatty alcohol acetates (fatty alcohol acetate esters, or FACE), fatty aldehydes, fatty amines, fatty amides, omega-hydroxy (ω-hydroxy) fatty esters, ω-hydroxy fatty acids, omega-carboxy (ω-carboxy) fatty esters, alpha,omega-diols (α,ω-diols), alpha,omega-fatty diacids (α,ω-fatty diacids), and/or alpha,omega-fatty diesters (α,ω-fatty diesters), and a reduced amount of byproducts, such as 3-hydroxy fatty acids and derivatives thereof and/or 3-oxo fatty acids and derivatives thereof, compared to an otherwise identical (or an otherwise isogenic, or a corresponding) recombinant cell, microorganism, or microbe that lacks i) one of a heterologous R-3-hydroxy acyl-CoA dehydratase or heterologous R-specific enoyl-CoA hydratase, and optionally also lacks ii) a heterologous trans-2-enoyl-CoA reductase; or that lacks i) one of a heterologous 3-oxoacyl-CoA reductase, a heterologous-ketoacyl-CoA-reductase, or a heterologous 3-hydroxy acyl-CoA dehydrogenase; ii) one of a heterologous R-3-hydroxy acyl-CoA dehydratase or heterologous R-specific enoyl-CoA hydratase, and optionally also lacks iii) a heterologous trans-2-enoyl-CoA reductase. In other words, the recombinant cell, microorganism, or microbe can produce, or is capable of producing, one or more fatty acids and/or fatty acid derivatives, or a composition comprising one or more fatty acids and/or derivatives thereof, and a reduced amount of byproducts, such as 3-hydroxy fatty acids and derivatives thereof and/or 3-oxo fatty acids and derivatives thereof, compared to an otherwise identical (or an otherwise isogenic, or a corresponding) recombinant cell, microorganism, or microbe that lacks a bypass pathway as described herein. The fatty acids and/or derivatives thereof, produced by the bypass pathways, methods, and the recombinant cells or microbes provided herein, can be a saturated or unsaturated, e.g., monounsaturated, even-chain or odd-chain, and/or branched-chain or straight-chain, C6-C20, e.g., C6, C7, C8, C9, C10, C11, C12, C13, C14, C15, C16, C17, C18, C19, or C20 fatty acid and/or derivative thereof. Any of the monounsaturated free fatty acids or monounsaturated fatty acid derivatives can contain a double bond at the omega-3 (ω-3), omega-5 (ω-5), omega-7 (ω-7), omega-9 (ω-9), or omega-11 (ω-11) position.

Also described herein are methods for producing one or more fatty acids and/or fatty acid derivatives, or for producing a composition comprising one or more fatty acids and/or derivatives thereof, the methods comprising: culturing, on a carbon source, a recombinant cell, microorganism, or microbe containing one or more of the enzymes described above. For example, provided herein is a method for producing one or more fatty acids and/or fatty acid derivatives, or for producing a composition comprising one or more fatty acids and/or derivatives thereof, the method comprising culturing, on a carbon source, a recombinant cell, microorganism, or microbe comprising one or more of i) a heterologous R-3-hydroxy-acyl-CoA dehydratase or a heterologous R-specific enoyl-CoA hydratase, and optionally, ii) a heterologous trans-2-enoyl-CoA reductase. The recombinant cell or microbe can also comprise one or more of fatty acid biosynthesis enzyme and/or one or more of a fatty acid derivative enzyme, which can be native or heterologous, or a combination thereof, and can be expressed or overexpressed, or a combination thereof. For example, the recombinant cell or microbe can additionally comprise an acyl-ACP thioesterase, an acyl-CoA synthetase, or a combination thereof, and/or can comprise any one or more of the other enzymes described above and elsewhere herein. The addition of a heterologous R-3-hydroxy acyl-CoA dehydratase or heterologous R-specific enoyl-CoA hydratase allows for the production of fatty acids and fatty acid derivatives in recombinant cells, microogranisms, or microbes, while reducing the amount of, or eliminating or substantially eliminating 3-hydroxy (3-OH) byproducts produced, compared to an otherwise isogenic recombinant cell, microorganism, or microbe. Additionally, a heterologous trans-2-enoyl-CoA reductase can also be co-expressed with the R-3-hydroxy acyl-CoA dehydratase or R-specific enoyl-CoA hydratase to reduce, eliminate, or substantially eliminate the production of 3-OH byproducts.

Thus, also provided herein are methods for reducing the amount of 3-hydroxy fatty acid and/or fatty acid derivative byproducts produced by a recombinant cell, microorganism, or microbe, the methods comprising culturing, on a carbon source, a recombinant cell, microorganism, or microbe comprising one or more of i) a heterologous R-3-hydroxy-acyl-CoA dehydratase or a heterologous R-specific enoyl-CoA hydratase, and optionally, ii) a heterologous trans-2-enoyl-CoA reductase. Also provided herein are methods for preparing a fatty acid and/or fatty acid derivative composition, comprising a reduced amount of 3-hydroxy fatty acid and/or fatty acid derivative byproducts, or for preparing a fatty acid and/or fatty acid derivative composition that is free or substantially free of 3-hydroxy fatty acid and/or fatty acid derivative byproducts, the methods comprising, culturing, on a carbon source, a recombinant cell, microorganism, or microbe comprising one or more of i) a heterologous R-3-hydroxy-acyl-CoA dehydratase or a heterologous R-specific enoyl-CoA hydratase, and optionally, ii) a heterologous trans-2-enoyl-CoA reductase.

Also provided herein is a method for producing one or more fatty acids and/or fatty acid derivatives, or for producing a composition comprising one or more fatty acids and/or derivatives thereof, the method comprising culturing, on a carbon source, a recombinant cell, microorganism, or microbe comprising one or more of i) a heterologous 3-oxoacyl-CoA reductase, a heterologous β-ketoacyl-CoA-reductase, or a heterologous 3-hydroxy acyl-CoA dehydrogenase; ii) a heterologous R-3-hydroxy-acyl-CoA dehydratase or a heterologous R-specific enoyl-CoA hydratase, and optionally, iii) a heterologous trans-2-enoyl-CoA reductase. The recombinant cell or microbe can also comprise one or more of fatty acid biosynthesis enzyme and/or one or more of a fatty acid derivative enzyme, which can be native or heterologous, or a combination thereof, and can be expressed or overexpressed, or a combination thereof. For example, the recombinant cell or microbe can additionally comprise an acyl-ACP thioesterase, an acyl-CoA synthetase, or a combination thereof, and/or can comprise any one or more of the other enzymes described above and elsewhere herein. The addition of a heterologous 3-oxoacyl-CoA reductase, a heterologous β-ketoacyl-CoA-reductase, or a heterologous 3-hydroxy acyl-CoA dehydrogenase; and a heterologous R-3-hydroxy acyl-CoA dehydratase or a heterologous R-specific enoyl-CoA hydratase, allows for the production of fatty acids and fatty acid derivatives in recombinant cells, microogranisms, or microbes, while reducing the amount of, or eliminating or substantially eliminating 3-hydroxy fatty acid and 3-oxo fatty acid derivative byproducts produced, compared to an otherwise isogenic recombinant cell, microorganism, or microbe. Additionally, a heterologous trans-2-enoyl-CoA reductase can also be expressed to reduce, eliminate, or substantially eliminate the production of 3-hydroxy and/or 3-oxo group containing byproducts.

Thus, also provided herein are methods for reducing the amount of 3-hydroxy and/or 3-oxo fatty acid and/or fatty acid derivative byproducts produced by a recombinant cell, microorganism, or microbe, the methods comprising culturing, on a carbon source, a recombinant cell, microorganism, or microbe comprising one or more of i) a heterologous 3-oxoacyl-CoA reductase, a heterologous-ketoacyl-CoA-reductase, or a heterologous 3-hydroxy acyl-CoA dehydrogenase; ii) a heterologous R-3-hydroxy-acyl-CoA dehydratase or a heterologous R-specific enoyl-CoA hydratase, and optionally, iii) a heterologous trans-2-enoyl-CoA reductase. Also provided herein are methods for preparing a fatty acid and/or fatty acid derivative composition, comprising a reduced amount of 3-hydroxy and/or 3-oxo fatty acid and/or fatty acid derivative byproducts, or for preparing a fatty acid and/or fatty acid derivative composition that is free or substantially free of 3-hydroxy and/or 3-oxo fatty acid and/or fatty acid derivative byproducts, the methods comprising, culturing, on a carbon source, a recombinant cell, microorganism, or microbe comprising one or more of i) a heterologous 3-oxoacyl-CoA reductase, a heterologous β-ketoacyl-CoA-reductase, or a heterologous 3-hydroxy acyl-CoA dehydrogenase; ii) a heterologous R-3-hydroxy-acyl-CoA dehydratase or a heterologous R-specific enoyl-CoA hydratase, and optionally, iii) a heterologous trans-2-enoyl-CoA reductase.

Also provided herein are compositions (e.g., fatty acid compositions and/or fatty acid derivative compositions), with a reduced amount of 3-hydroxy fatty acid byproducts, and/or 3-hydroxy fatty acid derivative byproducts, and/or 3-oxo fatty acid byproducts, and/or 3-oxo fatty acid derivative byproducts. In some embodiments, provided herein are compositions (e.g., fatty acid compositions and/or fatty acid derivative compositions), that are free or substantially free of 3-hydroxy fatty acid byproducts, and/or 3-hydroxy fatty acid derivative byproducts, and/or 3-oxo fatty acid byproducts, and/or 3-oxo fatty acid derivative byproducts. For example, the compositions provided herein, that are produced by the biosynthetic pathways (including the bypass pathways), methods, and recombinant cells, microbes, or microorganisms, provided herein, can contain about 0.01%, 0.1%, 0.2%, 0.3%, 0.4%, 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, less, by weight of the total composition, of 3-OH group containing byproducts (and/or trans-2 fatty acid and trans-2-fatty acid derivative byproducts), compared to a composition produced by a control or reference cell, microbe, or microorganism, that does not contain the R-3-hydroxy acyl-CoA dehydratase, R-specific enoyl-CoA hydratase, and/or trans-2-enoyl-CoA reductase. In some embodiments, the composition can comprise about 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold, less, 3-OH group containing byproducts (and/or trans-2 fatty acid and trans-2-fatty acid derivative byproducts), compared to a composition produced by a control or reference cell, microbe, or microorganism, that does not contain the R-3-hydroxy acyl-CoA dehydratase, R-specific enoyl-CoA hydratase, and/or trans-2-enoyl-CoA reductase. In certain embodiments, the compositions provided herein are free or substantially free of, 3-OH group containing byproducts, compared to a composition produced by a control or reference cell, microbe, or microorganism, that does not contain the R-3-hydroxy acyl-CoA dehydratase, R-specific enoyl-CoA hydratase, and/or trans-2-enoyl-CoA reductase.

Also provided herein are fatty acid and/or fatty acid derivative compositions that contain about 0.01%, 0.1%, 0.2%, 0.3%, 0.4%, 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, less, by weight of the total composition, of 3-oxo and/or 3-hydroxy group containing byproducts (and/or trans-2 fatty acid and trans-2-fatty acid derivative byproducts), compared to a composition produced by a control or reference cell, microbe, or microorganism, that does not contain the 3-oxoacyl-CoA reductase, β-ketoacyl-CoA-reductase, 3-hydroxy acyl-CoA dehydrogenase, R-3-hydroxy acyl-CoA dehydratase, R-specific enoyl-CoA hydratase, and/or trans-2-enoyl-CoA reductase. In some embodiments, the composition can comprise about 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold, less, 3-oxo and/or 3-hydroxy group containing byproducts (and/or trans-2 fatty acid and trans-2-fatty acid derivative byproducts), compared to a composition produced by a control or reference cell, microbe, or microorganism, that does not contain the 3-oxoacyl-CoA reductase, β-ketoacyl-CoA-reductase, 3-hydroxy acyl-CoA dehydrogenase, R-3-hydroxy acyl-CoA dehydratase, R-specific enoyl-CoA hydratase, and/or trans-2-enoyl-CoA reductase. In certain embodiments, the compositions provided herein are free or substantially free of, 3-oxo and/or 3-hydroxy group containing byproducts, compared to a composition produced by a control or reference cell, microbe, or microorganism, that does not contain the 3-oxoacyl-CoA reductase, β-ketoacyl-CoA-reductase, 3-hydroxy acyl-CoA dehydrogenase, R-3-hydroxy acyl-CoA dehydratase, R-specific enoyl-CoA hydratase, and/or trans-2-enoyl-CoA reductase.

The fatty acids and derivatives thereof, and/or the compositions comprising the fatty acids and derivatives thereof, produced by the recombinant cells, microorganisms, or microbes provided herein, or by the bypass pathways provided herein, or by the methods provided herein, can also be isolated and purified, and can be used in compositions to make ingredients or products, such as fragrances, flavors, pheromones, fuels, nutritional supplements, dietary supplements, pharmaceuticals, and/or nutraceuticals, and/or precursors thereof.

### BRIEF DESCRIPTION OF THE DRAWINGS

FIG. 1 depicts a fatty acid biosynthetic pathway which leads to the production of byproducts, such as 3-hydroxy fatty acids and derivatives thereof.

FIG. 2 depicts an example of a fatty acid biosynthetic pathway (e.g, a “bypass pathway”) comprising a heterologous R-3-hydroxy-acyl-CoA dehydratase or a heterologous R-specific enoyl-CoA hydratase, and optionally, a heterologous trans-2-enoyl-CoA reductase. This biosynthetic pathway promotes the production of non-3-hydroxy fatty acid derivatives (i.e., fatty acids and derivatives thereof that do not contain a 3-hydroxy group), with a reduced generation of byproducts, such as 3-hydroxy fatty acids and derivatives thereof.

FIG. 3 depicts an exemplary fatty acid biosynthetic pathway (e.g, a “bypass pathway”) comprising i) a heterologous 3-oxoacyl-CoA reductase, a heterologous β-ketoacyl-CoA-reductase, or a heterologous 3-hydroxy acyl-CoA dehydrogenase; ii) a heterologous R-3-hydroxy-acyl-CoA dehydratase or a heterologous R-specific enoyl-CoA hydratase; and optionally, iii) a heterologous trans-2-enoyl-CoA reductase. This biosynthetic pathway promotes the production of non-3-oxo and non-3-hydroxy fatty acid derivatives (i.e., fatty acids and derivatives thereof that do not contain a 3-oxo group or a 3-hydroxy group), with a reduced generation of byproducts, such as 3-oxo and 3-hydroxy fatty acids and derivatives thereof.

FIG. 4 depicts the GC chromatograph of the fatty acid ethyl ester composition produced by strain sven. 1169.

FIG. 5 depicts the ion fragmentation pattern (inset) and the GS/MS chromatograph for the peak (from FIG. 4; retention time of approximately 6.328 minutes) corresponding to 3-oxo-octanoic acid ethyl ester (3-keto-octanoic acid ethyl ester).

### DETAILED DESCRIPTION

### I. Definitions

The following definitions refer to the various terms used above and throughout the disclosure.

As used herein, singular articles such as “a” and “an” and “the” and similar referents in the context of describing the elements are to be construed to cover both the singular and the plural, unless otherwise indicated herein or clearly contradicted by context.

As used herein, the term “or” is used to mean “and/or” unless explicitly indicated to refer to alternatives only or if the alternatives are mutually exclusive.

As used herein, “about” is understood by persons of ordinary skill in the art and may vary to some extent depending upon the context in which it is used. If there are uses of the term which are not clear to persons of ordinary skill in the art given the context in which the term “about” is used, “about” will mean up to plus or minus 10% of the particular term. As used herein, ranges and amounts can be expressed as “about” a particular value or range. About also includes the exact amount. Hence, “about 10%” means “about 10%” and also means “10%.”

As used herein, “optional” or “optionally” means that the subsequently described event or circumstance does or does not occur, and that the description includes instances where said event or circumstance occurs and instances where it does not. For example, an optionally expressed polypeptide means that the polypeptide is expressed or is not expressed.

As will be understood by one skilled in the art, for any and all purposes, all ranges disclosed herein also encompass any and all possible subranges and combinations of subranges thereof. Furthermore, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Unless defined otherwise, technical and scientific terms used herein have the same meaning as is commonly understood by a person of ordinary skill in the art. In particular, this disclosure utilizes routine techniques in the field of recombinant genetics, organic chemistry, fermentation, and biochemistry.

As used herein, an “acyl-ACP thioesterase” or “a polypeptide with acyl-ACP thioesterase activity” refers to a polypeptide or enzyme that catalyzes or is capable of catalyzing the hydrolysis of thioester bonds in fatty acyl-ACPs to terminate fatty acyl extension and generate free fatty acids. In other words, an acyl-ACP thioesterase or a polypeptide with acyl-ACP thioesterase activity is capable of converting or hydrolyzing an acyl-ACP to a free fatty acid. The acyl-ACP thioesterase can be described by the number EC 3.1.2.14 or EC 3.1.2.21, and can also be referred to as an acyl-ACP hydrolase.

The term “fatty acid” or “free fatty acid” as used herein, refers to an aliphatic carboxylic acid having the formula RCOOH, wherein R is an aliphatic group having at least 4 carbons, typically between about 4 and about 28 carbon atoms. The aliphatic R group can be saturated or unsaturated, and/or can be branched or unbranched. Branched aliphatic R groups may include branches comprising lower alkyl branches, such as a C1-C4 alkyl, preferably in an ω-1 or ω-2 position. In some embodiments, the branched aliphatic R group may be a methyl group in the ω-1 or ω-2 position. Unsaturated fatty acids may be monounsaturated or polyunsaturated. A “3-hydroxy fatty acid” refers to a fatty acid with a hydroxy (OH) group in the 3 position, where the carboxyl group carbon is assigned position number 1. A “3-hydroxy” or “3-OH” fatty acid or fatty acid derivative can also be referred to as a “beta-hydroxy,” “beta-OH”, or “β-hydroxy” or “β-OH” fatty acid or fatty acid derivative. A “3-oxo fatty acid” refers to a fatty acid with an oxo (or keto/ketone) group in the 3 position, where the carboxyl group carbon is assigned position number 1. A “3-oxo” fatty acid or fatty acid derivative can also be referred to as a “beta-oxo”, “B-oxo”, “beta-keto”, or “B-keto” fatty acid or fatty acid derivative.

The term “omega” or “@” as used herein, with respect to positioning within the carbon chain, refers to the last carbon in the chain, farthest from the carboxyl group, in a fatty acid or fatty acid derivative, or farthest from the thioester group, for example, in a fatty acyl-CoA or fatty acyl-ACP molecule. When a number is appended to the term “omega” or “ω,” that number denotes the position with respect to the omega carbon. For example, a substituent at the omega-1 (ω-1) position is attached to the penultimate carbon. For example, a C12 fatty acid, with a hydroxy group at the ω position can be referred to as 12-hydroxy dodecanoic acid; a C12 fatty acid with a hydroxy group at the ω-1 position can be referred to as 11-hydroxy dodecanoic acid; a C12 fatty acid with a hydroxy group at the ω-2 position can be referred to as 10-hydroxy dodecanoic acid, and so forth. The omega (ω) numbering of the double bond position in a compound does not indicate the geometric isomerism of the compound; thus, as used herein, ω7-hexadecenoic acid can have a cis or a trans double bond, or the term may refer to a mixture of cis and trans isomers thereof.

The position of a double bond within a carbon chain in any of the fatty acids or derivatives thereof provided herein also can be described by the upper-case Greek letter “Δ”, or “delta”, followed by a number, which refers to the position of the double bond with respect to the carboxyl group (in a fatty acid or derivative thereof), or with respect to the thioester group (in a fatty acyl-CoA or fatty acyl-ACP), where the carbon of the carboxyl or thioester group is designated as position number 1. For example, Δ9-hexadecenoic acid refers to a C16 fatty acid containing a double bond between carbon numbers 9 and 10, where the carboxyl carbon is at position number 1. Similarly, Δ7-hexadecenoic acid has a double bond between carbon numbers 7 and 8, with the carboxyl carbon having position number 1. Δ7-hexadecenoic acid and Δ9-hexadecenoic acid can also be referred to as ω9-hexadecenoic acid and ω7-hexadecenoic acid, respectively. The delta (Δ) numbering of the double bond position in a compound does not indicate the geometric isomerism of the compound; thus, as used herein, Δ9-hexadecenoic acid can refer to Z9-hexadecenoic acid (or cis-9- or (9Z)-hexadecenoic acid), or to E9-hexadecenoic acid (or trans-9- or (9E)-hexadecenoic acid), or to a mixture thereof.

The fatty acids and derivatives thereof provided herein can be described in terms of their geometric isomerism. Geometric isomers can be represented by the symbol which denotes a bond that can be a single, double, or triple bond as described herein. Provided herein are various geometric isomers and mixtures thereof resulting from the arrangement of substituents around a carbon-carbon double bond. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

The term “fatty acid derivative” as used herein, refers to a product derived from a fatty acid, or from a fatty acyl thioester, such as a fatty acyl-ACP or a fatty acyl-CoA. Thus, a fatty acid derivative can refer to a compound that includes a fatty acid as defined above with a modification. In general, fatty acid derivatives include malonyl-CoA derived compounds, including acyl-ACP or acyl-CoA derivatives. Thus, a fatty acid derivative includes alkyl-thioesters and acyl-thioesters. Further, a fatty acid derivative includes a molecule or compound that is derived from a metabolic pathway that includes a fatty acid derivative enzyme. Exemplary fatty acid derivatives include, but are not limited to, for example, fatty acids, fatty acid esters (e.g., waxes), fatty acid alkyl esters, fatty acid methyl esters (FAME), fatty acid ethyl esters (FAEE), fatty alcohol acetate esters (FACE; also referred to herein as fatty alcohol acetates), fatty amines, fatty amides, fatty acetates, fatty aldehydes, fatty alcohols, hydrocarbons (e.g., alkanes, alkenes, etc.), ketones, terminal olefins, internal olefins, 3-hydroxy fatty acid derivatives, bifunctional fatty acid derivatives (e.g., w-hydroxy fatty acids, (ω-1)-hydroxy fatty acids, (ω-2)-hydroxy fatty acids, (ω-3)-hydroxy fatty acids, ω-hydroxy fatty esters, ω-carboxy fatty esters, α,ω-fatty diacids, α,ω-fatty diesters, 1,3 fatty diols, α,ω-diols, α,ω-3-hydroxy triols, ω-hydroxy FAME, ω-OH FAEE, etc.), and unsaturated fatty acid derivatives, including unsaturated versions of each of the above mentioned fatty acid derivatives. The fatty acid derivatives can be saturated or unsaturated, and/or can be branched or unbranched. Unsaturated fatty acid derivatives can be monounsaturated or polyunsaturated. The fatty acid derivative typically contains between about 4 and about 28 carbon atoms, for example, 4, 5, 6, 7, 8, 9, 10, 11, 12, 13, 14, 15, 16, 17, 18, 19, 20, 21, 22, 23, 24, 25, 26, 27, or 28 carbon atoms. A fatty acid alkyl ester can be a methyl, ethyl, propyl, isopropyl, butyl, isobutyl, or other alkyl ester.

The fatty acids or fatty acid derivatives, as used herein, can be produced within a cell through the process of fatty acid biosynthesis, through the reverse of fatty acid degradation or beta (β)-oxidation, or they can be fed to a cell. As is well known in the art, fatty acid biosynthesis is generally a malonyl-CoA dependent synthesis of acyl-ACPs or acyl-CoAs, while the reverse of beta-oxidation is acetyl-CoA dependent and results in the synthesis of acyl-CoAs. Fatty acids fed to cells are converted to acyl-CoAs and can be converted to acyl-ACPs. Fatty acids can be synthesized in a cell by natural (i.e., native or endogenous) fatty acid biosynthetic pathways, or can be synthesized from heterologous fatty acid biosynthetic pathways, that comprise a combination of fatty acid biosynthetic and/or degradation enzymes that result in the synthesis of acyl-CoAs and/or acyl-ACPs.

The term “malonyl-CoA derived compound” as used herein refers to any compound or chemical entity (i.e., intermediate or end product) that is made via a biochemical pathway wherein malonyl-CoA functions as an intermediate and/or is made upstream of the compound or chemical entity. For example, a malonyl-CoA derived compound may include, but is not limited to, a fatty acid derivative such as, for example, a fatty acid; a fatty ester including, but not limited to a fatty acid methyl ester (FAME) and/or a fatty acid ethyl ester (FAEE); a fatty alcohol; a fatty aldehyde; a fatty amine; a fatty amide; an alkane; an olefin or alkene; a hydrocarbon; a bifunctional fatty acid derivative; a multifunctional fatty acid derivative; a native or non-native unsaturated fatty acid derivative, etc.

As used herein, an “alkyl-thioester” or equivalently an “acyl thioester” is a compound in which the carbonyl carbon of an acyl chain and the sulfhydryl group of an organic thiol are joined through a thioester bond. Representative organic thiols include, e.g., cysteine, beta-cysteine, glutathione, mycothiol, pantetheine, Coenzyme A (CoA), and the acyl carrier protein (ACP). An “acyl-ACP” refers to an “alkyl-thioester” formed between the carbonyl carbon of an acyl chain and the sulfhydryl group of the phosphopantetheinyl moiety of an ACP. An “acyl-CoA” refers to an “alkyl-thioester” formed between the carbonyl carbon of an acyl chain and the sulfhydryl group of the phosphopantetheinyl moiety of CoA. In some embodiments an “alkyl-thioester”, such as an acyl-ACP or an acyl-CoA, is an intermediate in the synthesis of fully saturated acyl thioesters. In other embodiments, an “alkyl-thioester”, such as an acyl-ACP or an acyl-CoA, is an intermediate in the synthesis of unsaturated acyl thioesters. In some embodiments, the carbon chain of the acyl group of an acyl thiester has 5, 6, 7, 8, 9, 10, 11, 12, 13, 14, 15, 16, 17, 18, 19, 20, 21, 22, 23, 24, 25, 26, 27, or 28 carbons. In other embodiments, the carbon chain of the acyl group of an acyl thioester is a medium-chain and has 6, 7, 8, 9, 10, 11, 12, 13, 14, 15, 16, 17, or 18 carbons. In other exemplary embodiments the carbon chain of the acyl group of an acyl-thioester is 8 carbons in length. In other exemplary embodiments the carbon chain of the acyl group of an acyl-thioester is 10 carbons in length. In still other exemplary embodiments, the carbon chain of the acyl group of an acyl-thioester is 12 carbons in length. In still other exemplary embodiments, the carbon chain of the acyl group of an acyl-thioester is 14 carbons in length. In still other exemplary embodiments, the carbon chain of the acyl group of an acyl-thioester is 16 carbons in length. Alkyl-thioesters are substrates for fatty acid derivative enzymes, such as, e.g., lactonizing enzymes, thioesterases, acyl-ACP reductases, acyl-CoA reductases, and ester synthases, and their engineered variants, that convert the acyl-thioester to fatty acid derivatives such, as e.g., natural lactones, fatty acids, fatty aldehydes, or fatty esters.

As used herein, the term “acyl-ACP dependent fatty acid derivative biosynthetic pathway”, “acyl-ACP dependent biosynthetic/biosynthesis pathway” or “fatty acyl-ACP dependent biosynthetic/biosynthesis pathway” refers to a fatty acid derivative (or fatty acid) biosynthesis pathway wherein the alkyl-thioester or acyl-thioester intermediate compounds are acyl-ACPs.

As used herein, the term “acyl-CoA dependent fatty acid biosynthetic pathway,” “acyl-CoA dependent fatty acid derivative biosynthetic pathway,” or “fatty acyl-CoA dependent biosynthetic/biosynthesis pathway” or “acyl-CoA dependent biosynthetic/biosynthesis pathway” refers to a fatty acid derivative (or fatty acid) biosynthesis/biosynthetic pathway wherein the alkyl-thioester or acyl-thioester intermediate compounds are acyl-CoAs.

As used herein, the expression “fatty acid derivative biosynthetic/biosynthesis pathway” refers to a biochemical pathway that produces fatty acid derivatives. The enzymes that comprise a “fatty acid derivative biosynthetic/biosynthesis pathway” are thus referred to herein as “fatty acid derivative biosynthetic/biosynthesis polypeptides” or equivalently “fatty acid derivative enzymes.” As discussed supra, and elsewhere herein, the term “fatty acid derivative” includes a molecule or compound derived from a biochemical pathway that includes a fatty acid derivative enzyme. Thus, a thioesterase enzyme (e.g., an enzyme having thioesterase activity, such as EC 3.2.1.14) is a “fatty acid derivative biosynthetic/biosynthesis polypeptide” or equivalently, a “fatty acid derivative enzyme.” Thus, the term “fatty acid derivative enzymes” or equivalently “fatty acid derivative biosynthetic/biosynthesis polypeptides” refers, collectively and individually, to enzymes that may be expressed or overexpressed (e.g., in a host cell, microbe, or microorganism) to produce fatty acid derivatives, such as, e.g., a fatty acid methyl ester (FAME) or a fatty acid ethyl ester (FAEE). Additional non-limiting examples of “fatty acid derivative enzymes” or equivalently “fatty acid derivative biosynthetic/biosynthesis polypeptides” include, e.g., fatty acid synthases, lactonizing enzymes, thioesterases, acyl-CoA synthetases, acyl-CoA reductases, acyl-ACP reductases, alcohol dehydrogenases, alcohol oxidases, aldehyde dehydrogenases, alcohol O-acyltransferases, fatty alcohol-forming acyl-CoA reductases, fatty acid decarboxylases, fatty aldehyde decarbonylases and/or oxidative deformylases, carboxylic acid reductases, fatty alcohol O-acetyl transferases, hydroxylating enzymes (including, for example omega-hydroxylases, oxygenases, or monooxygenases), hydratases, desaturases, ester synthases, transaminases (aminotransferases), etc. “Fatty acid derivative enzymes” or equivalently “fatty acid derivative biosynthetic/biosynthesis polypeptides” convert substrates into fatty acid derivatives. The substrate for a fatty acid derivative enzyme can be an intermediate of a fatty acid derivative biosynthetic/biosynthesis pathway. For example, a fatty acyl-ACP can be a substrate for a thioesterase, which converts the acyl-ACP to a free fatty acid, and the free fatty acid (as an intermediate), in turn, can be a substrate for a carboxylic acid reductase, which converts the fatty acid to a fatty aldehyde. Further, the fatty aldehyde can act as an intermediate, and can be a substrate for an alcohol dehydrogenase, which converts the fatty aldehyde intermediate into a fatty alcohol product. The expression “fatty acid composition” or “fatty acid derivative composition” as used herein, refers to a composition of fatty acids and/or fatty acid derivatives thereof, that contain one or more fatty acids and/or fatty acid derivatives. For example, a fatty acid or fatty acid derivative composition produced by the recombinant cells, microorganisms, or microbes described herein, such as a recombinant proteobacterium, comprising one or more of i) a heterologous 3-oxoacyl-CoA reductase, heterologous β-ketoacyl-CoA-reductase, or heterologous 3-hydroxy acyl-CoA dehydrogenase, ii) a heterologous R-3-hydroxy-acyl-CoA dehydratase or a heterologous R-specific enoyl-CoA hydratase, and, optionally, iii) a heterologous trans-2-enoyl-CoA reductase. A fatty acid composition or a fatty acid derivative composition can comprise a single fatty acid derivative species or may comprise a mixture of fatty acid derivative species. In some exemplary embodiments, the mixture of fatty acid derivatives includes more than one type of fatty acid derivative product (e.g., fatty acids, fatty acid esters, fatty alcohols, fatty alcohol acetates, fatty aldehydes, fatty amines, bifunctional fatty acid derivatives, and non-native monounsaturated fatty acid derivatives, etc.). In other exemplary embodiments, the mixture of fatty acid derivatives includes a mixture of fatty acid alkyl esters (or another fatty acid derivative(s)) with different chain lengths, saturation and/or branching characteristics. In other exemplary embodiments, the mixture of fatty acid derivatives comprises predominantly one type of fatty acid derivative e.g., a fatty acid methyl ester or a fatty acid ethyl ester. In still other exemplary embodiments, a fatty acid derivative composition comprises a mixture of more than one type of fatty acid derivative product e.g., fatty acid derivatives with different chain lengths, saturation and/or branching characteristics. In some embodiments, a “fatty acid composition” or a “fatty acid derivative composition” comprises a mixture of fatty acids and/or fatty acid derivatives, and little to no 3-hydroxy fatty acids and/or derivatives thereof, and/or little to no 3-oxo fatty acids and/or derivatives thereof. In still other exemplary embodiments, a “fatty acid derivative composition” comprises a mixture of fatty esters and little to no 3-hydroxy fatty esters and/or 3-oxo fatty esters. In still other exemplary embodiments, a fatty acid derivative composition comprises a mixture of fatty acids and fatty esters; or a mixture of fatty alcohols and fatty aldehydes; or a mixture of saturated and monounsaturated fatty acids and/or fatty esters; or a mixture of saturated and monounsaturated fatty alcohols and/or fatty aldehydes. In other exemplary embodiments, the mixture of fatty acid derivatives includes a mixture of fatty acid derivatives with different chain lengths, saturation levels, branching characteristics, and/or functional group characteristics.

Sequence Accession numbers throughout this description were obtained from databases provided by the NCBI (National Center for Biotechnology Information) maintained by the National Institutes of Health, U.S.A. (which are identified herein as “NCBI Accession Numbers” or alternatively as “GenBank Accession Numbers” or alternatively as simply “Accession Numbers”), and from the UniProt Knowledgebase (UniProtKB) and Swiss-Prot databases, provided by the Swiss Institute of Bioinformatics (which are identified herein as “UniProtKB Accession Numbers”).

The term “enzyme classification (EC) number” refers to a number that denotes a specific polypeptide sequence or enzyme. EC numbers classify enzymes according to the reaction they catalyze. EC numbers are established by the nomenclature committee of the international union of biochemistry and molecular biology (IUBMB), a description of which is available on the IUBMB enzyme nomenclature website on the world wide web.

As used herein, the terms “isolated” and “purified,” with respect to products (such as fatty acids and derivatives thereof disclosed herein), refers to products that are separated from cellular components, cell culture media, fermentation broth, and/or chemical or synthetic precursors. The fatty acids and derivatives thereof disclosed herein, produced by cells, microbes, cell cultures, and/or the methods disclosed herein, can be relatively immiscible in the fermentation broth, as well as in the cytoplasm. Therefore, in exemplary embodiments, the fatty acids and derivatives thereof disclosed herein collect in an organic phase extracellularly and are thereby “isolated”.

As used herein, the terms “polypeptide” and “protein” are used interchangeably to refer to a polymer of amino acid residues that is typically 12 or more amino acids in length. Polypeptides less than 12 amino acids in length are referred to herein as “peptides.” The terms apply to amino acid polymers in which one or more amino acid residue is an artificial chemical mimetic of a corresponding naturally occurring amino acid, as well as to naturally occurring amino acid polymers and non-naturally occurring amino acid polymers. The term “recombinant polypeptide” refers to a polypeptide that is produced by recombinant techniques, wherein generally DNA or RNA encoding the expressed protein is inserted into a suitable expression vector that is in turn used to transform a host cell to produce the polypeptide. In some exemplary embodiments, DNA or RNA encoding an expressed peptide, polypeptide, or protein is inserted into the host chromosome via homologous recombination or other means well known in the art and is so used to transform a host cell to produce the peptide or polypeptide. Similarly, the terms “recombinant polynucleotide” or “recombinant nucleic acid” or “recombinant DNA” are produced by recombinant techniques that are known to those of skill in the art (see e.g., methods described in Sambrook et al. (Sambrook et al., Molecular Cloning—A Laboratory Manual, Cold Spring Harbor Press 4th Edition (Cold Spring Harbor, N.Y. 2012) and/or Current Protocols in Molecular Biology (Volumes 1-3, John Wiley & Sons, Inc. (1994-1998) and Supplements 1-115 (1987-2016).).

As use herein, “sequence identity” refers to the number of identical amino acids (or nucleotide bases) in a comparison between a test and a reference polypeptide or polynucleotide. When referring to two nucleotide or polypeptide sequences, the “percentage of sequence identity” between the two sequences is determined by comparing the two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The “percentage of sequence identity” is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Thus, the expression “percent identity,” or equivalently “percent sequence identity,” “homology, or “homologous” in the context of two or more nucleic acid sequences or peptides or polypeptides, refers to two or more sequences or subsequences that are the same or have a specified percentage of nucleotides or amino acids that are the same (e.g., about 50% identity, preferably 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured e.g., using a BLAST or BLAST 2.0 sequence comparison algorithm with default parameters (see e.g., Altschul et al. (1990) J. Mol. Biol. 215 (3): 403-410) and/or the NCBI web site at ncbi.nlm.nih.gov/BLAST/) or by manual alignment and visual inspection. Percent sequence identity between two nucleic acid or amino acid sequences also can be determined using e.g., the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6 (Needleman and Wunsch (1970) J. Mol. Biol. 48:444-453). The percent sequence identity between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial sequence identity calculations and adjust the algorithm parameters accordingly. A set of parameters that may be used if a practitioner is uncertain about which parameters should be applied to determine if a molecule is within a sequence identity limitation of the claims, are a Blossum 62 scoring matrix with a gap penalty of 12, a gap extend penalty of 4, and a frameshift gap penalty of 5. Additional methods of sequence alignment are known in the biotechnology arts (see, e.g., Rosenberg (2005) BMC Bioinformatics 6:278; Altschul et al. (2005) FEBS J. 272 (20): 5101-5109).

Two or more nucleic acid or amino acid sequences are said to be “substantially identical,” when they are aligned and analyzed as discussed above and are found to share about 50% identity, preferably 55%, 60%, 65%, 70%, 75%, 80%, 85%, 90%, 91%, 92%, 93%, 94%, 95%, 96%, 97%, 98%, 99%, or higher identity over a specified region. Two nucleic acid sequences or polypeptide sequences are said to be “identical” if the sequence of nucleotides or amino acid residues, respectively, in the two sequences are the same when aligned for maximum correspondence as described above. This definition also refers to, or may be applied to, the compliment of a test sequence. Identity is typically calculated over a region that is at least about 25 amino acids or nucleotides in length, or more preferably over a region that is 50-100 amino acids or nucleotides in length, or over the entire length of a given sequence.

The term “endogenous” as used herein refers to a substance e.g., a nucleic acid, protein, enzyme, etc. that is produced from within a cell and/or that is naturally occurring or naturally found inside a cell. Similarly, an endogenous pathway (such as a fatty acid biosynthesis pathway or a fatty acid derivative pathway) is one that is naturally occurring or naturally found inside a cell. Thus, an endogenous nucleic acid sequence, gene, polynucleotide, or polypeptide refers to a nucleic acid sequence, gene, polynucleotide, or polypeptide produced by and found inside the cell. In some exemplary embodiments an endogenous polypeptide or polynucleotide is encoded by the genome of the parental cell (or host cell). In other exemplary embodiments, an endogenous polypeptide or polynucleotide is encoded by an autonomously replicating plasmid carried by the parental cell (or host cell). In some exemplary embodiments, an endogenous gene or nucleic acid sequence is a gene or nucleic acid sequence that was present in the cell when the cell was originally isolated from nature, i.e., the gene is native to the cell.

In contrast, an “exogenous” nucleic acid sequence, gene, polynucleotide, or polypeptide (e.g., an enzyme), or other substance (e.g., fatty acid derivative, small molecule compound, etc.), as used herein, refers to a nucleic acid sequence, gene, polynucleotide, or polypeptide or other substance that is not encoded by or produced by the cell, and which is therefore added to a cell, a cell culture, or assay, from outside of the cell. A nucleic acid sequence encoding a variant (i.e., mutant) polypeptide, when added to the cell, is one example of an exogenous nucleic acid sequence. Similarly, a nucleic acid sequence encoding a fatty acid biosynthesis enzyme or fatty acid derivative enzyme, when introduced into a cell (e.g., in a vector, such as a plasmid), is considered an exogenous nucleic acid sequence. The exogenous nucleic acid sequence can encode a polypeptide or an enzyme that is also otherwise endogenous or native to the cell. Such an encoded polypeptide or enzyme can be considered “exogenously expressed.” For example, to achieve overexpression of an endogenous gene, additional copies of the gene can be introduced into the cell (e.g., in a vector, such as a plasmid); such additional copies of the endogenous gene can be considered as “exogenous” (e.g., exogenous gene(s) or an exogenous nucleic acid sequence(s)), because the additional copies are introduced into the cell from outside the cell. An “exogenous gene” or “exogenous nucleic acid sequence” also refers to a native (or endogenous) gene or nucleic acid sequence that is deregulated (e.g., upregulated or attenuated) or otherwise altered or modified, for example, by operably linking it to a regulatory element, such as a heterologous, or non-native, or non-naturally occurring, regulatory element (e.g., a promoter, enhancer, 5′-UTR, ribosome binding site, etc.); such a deregulated or altered gene or nucleic acid sequence can be on a chromosome or can be on a plasmid. An exogenous nucleic acid sequence or exogenous gene can also be used to express or overexpress a heterologous polypeptide or enzyme in a cell. Thus, an exogenous nucleic acid sequence or an exogenous gene can encode a polypeptide (e.g., an enzyme) that is native to the cell, that is otherwise endogenous to the cell, or that is heterologous to the cell.

The term “heterologous” as used herein refers to a polypeptide or polynucleotide which is in a non-native state. Thus, a polynucleotide or a polypeptide is “heterologous” to a cell when the polynucleotide and/or the polypeptide and the cell are not found in the same relationship to each other in nature. Therefore, a polynucleotide or polypeptide sequence is “heterologous” to an organism or a second sequence if it originates from a different organism, different cell type, or different species, or, if from the same species, it is modified from its original form. Thus, in an exemplary embodiment, a polynucleotide or polypeptide is “heterologous” when it is not naturally present in a given organism. For example, a polynucleotide sequence that is native to cyanobacteria can be introduced into a host cell of E. coli (a proteobacterium) by recombinant methods, and the polynucleotide from cyanobacteria is then heterologous to the E. coli cell (i.e., the now recombinant E. coli cell).

Similarly, a polynucleotide or polypeptide is heterologous when it is modified from its native form or from its relationship with other polynucleotide sequences or is present in a recombinant host cell in a non-native state. Thus, in an exemplary embodiment, a heterologous polynucleotide or polypeptide comprises two or more subsequences that are not found in the same relationship to each other in nature. For example, a promoter operably linked to a nucleotide coding sequence derived from a species different from that from which the promoter was derived. Alternatively, in another example, if a promoter is operably linked to a nucleotide coding sequence derived from a species that is the same as that from which the promoter was derived, then the operably-linked promoter and coding sequence are “heterologous” if the coding sequence is not naturally associated with the promoter (e.g. a constitutive promoter operably linked to a developmentally regulated coding sequence that is derived from the same species as the promoter). In other exemplary embodiments, a heterologous polynucleotide or polypeptide is modified relative to the wild type sequence naturally present in the corresponding wild type host cell, e.g., an intentional modification e.g., an intentional mutation in the sequence of a polynucleotide or polypeptide or a modification in the level of expression of the polynucleotide or polypeptide. Typically, a heterologous nucleic acid or polynucleotide is recombinantly produced. A heterologous polynucleotide, polypeptide, or enzyme, for example, is typically exogenous to the cell, or exogenously expressed (or overexpressed) in the cell, i.e., is introduced into or added to the cell from outside the cell.

As used herein the term “native” refers to the form of a nucleic acid, protein, polypeptide or a fragment thereof that is isolated from nature, or to a nucleic acid, protein, polypeptide or a fragment thereof that is in its natural state without intentionally introduced mutations in the structural sequence and/or without any engineered changes in expression such as e.g., changing a developmentally regulated gene to a constitutively expressed gene. As used herein, “native” also refers to “wildtype” or “wild-type,” in which the nucleic acid, protein, polypeptide, or a fragment thereof is present in both sequence, quantity, and relative quantity as typically found in the organism as naturally found. Wild-type organisms may serve as a control and/or reference for determination of cellular functions, such as to identity and/or quantity fatty acid(s) and derivatives thereof produced. A native gene, nucleic acid sequence, polypeptide, or enzyme, for example, is typically endogenous to a cell, i.e., found in or produced by the cell. An exogenous nucleic acid sequence or an exogenous gene can encode a native polypeptide or enzyme, for example, where additional copies of a native gene or nucleic acid sequence are added to the cell from outside the cell, or where a native gene or nucleic acid sequence is deregulated or altered, e.g., by operably coupling it to a regulatory element that is not native or endogenous to the cell.

The term “non-native” is used herein to refer to nucleic acid sequences, amino acid sequences, polypeptide sequences, enzymes, fatty acids and derivatives thereof, and/or small molecules that do not occur naturally in the host. Heterologous genes and polypeptides are considered “non-native.” A nucleic acid sequence or amino acid sequence that has been removed from a host cell, subjected to laboratory manipulation, and introduced or reintroduced into a host cell, is also considered “non-native.” Synthetic or partially synthetic genes introduced into a host cell are “non-native.” Non-native genes further include genes that are endogenous and/or native to the host microorganism but that are operably linked to one or more heterologous regulatory sequences that have been recombined into the host genome. A naturally occurring gene under the control of a heterologous regulatory sequence is considered “non-native.” In some embodiments, an organism comprising a non-native gene may be utilized as a control and/or reference for an organism having additional and/or different variations from wildtype organisms.

The term “gene” as used herein, refers to nucleic acid sequences e.g., DNA sequences, which encode either an RNA product or a protein product, as well as operably linked nucleic acid sequences that affect expression of the RNA or protein product (e.g., expression control sequences such as e.g., promoters, enhancers, ribosome binding sites, translational control sequences, etc). The term “gene product” refers to either the RNA (e.g., tRNA, mRNA) and/or protein expressed from a particular gene. Nucleic acid sequences can include those with degenerate codon sequences that encode the same product (i.e., polypeptide or protein).

The term “expression” or “expressed” as used herein in reference to a gene, refers to the production of one or more transcriptional and/or translational product(s) of a gene. In exemplary embodiments, the level of expression of a DNA molecule in a cell is determined on the basis of either the amount of corresponding mRNA that is present within the cell or the amount of protein encoded by that DNA produced by the cell. The term “expressed genes” refers to genes that are transcribed into messenger RNA (mRNA) and then translated into protein, as well as genes that are transcribed into other types of RNA, such as e.g., transfer RNA (tRNA), ribosomal RNA (rRNA), and regulatory RNA, which are not translated into protein.

The level of expression of a nucleic acid molecule in a cell or cell free system is influenced by “expression control sequences” or equivalently “regulatory sequences” or “regulatory elements.” Expression control sequences, regulatory sequences, or regulatory elements are known in the art and include, for example, promoters, enhancers, polyadenylation signals, transcription terminators, nucleotide sequences that affect RNA stability, internal ribosome entry sites (IRES), and the like, that provide for the expression of the polynucleotide sequence in a host cell. In exemplary embodiments, “expression control sequences” interact specifically with cellular proteins involved in transcription (see e.g., Maniatis et al., Science, 236:1237-1245 (1987); Goeddel, Gene Expression Technology: Methods in Enzymology, Vol. 185, Academic Press, San Diego, Calif. (1990)). In exemplary methods, an expression control sequence, regulatory sequence, or regulatory element is operably linked to a polynucleotide sequence. By “operably linked” is meant that a polynucleotide sequence and an expression control sequence(s) or regulatory element(s) are functionally connected so as to permit expression of the polynucleotide sequence when the appropriate molecules (e.g., transcriptional activator proteins) contact the expression control sequence(s). In exemplary embodiments, operably linked promoters are located upstream of the selected polynucleotide sequence in terms of the direction of transcription and translation. In some exemplary embodiments, operably linked enhancers can be located upstream, within, or downstream of the selected polynucleotide.

As used herein, the phrase “expression of said nucleotide sequence is modified relative to the wild-type nucleotide sequence,” refers to a change e.g., an increase or decrease in the level of expression of a native nucleotide sequence or a change e.g., an increase or decrease in the level of the expression of a heterologous or non-native polypeptide-encoding nucleotide sequence as compared to a control nucleotide sequence e.g., wild-type control. In some exemplary embodiments, the phrase “the expression of said nucleotide sequence is modified relative to the wild type nucleotide sequence,” refers to a change in the pattern of expression of a nucleotide sequence as compared to a control pattern of expression e.g., constitutive expression as compared to developmentally timed expression.

A “control” sample (e.g., a control nucleotide sequence, a control polypeptide sequence, a control cell, etc., or value) refers to a sample that serves as a reference, usually a known reference, for comparison to a test sample. For example, in an exemplary embodiment, a test sample comprises a fatty acid derivative composition made by a recombinant microbe that comprises a heterologous R-3-hydroxy-acyl-CoA dehydratase or heterologous R-specific enoyl-CoA hydratase, and optionally, a heterologous trans-2-enoyl reductase, while the control sample comprises a a fatty acid derivative composition made by the corresponding or designated microbe that does not comprise the combination of heterologous enzymes described herein. Additionally, a control cell or microorganism may be referred to as a corresponding wild type or host cell. One of skill will recognize that controls can be designed for assessment of any number of parameters. Furthermore, one of skill in the art will understand which controls are valuable in a given situation and will be able to analyze data based on comparisons to control values.

The term “overexpressed” or “up-regulated” as used herein, refers to a gene whose expression is elevated in comparison to a control level of expression. In exemplary embodiments, overexpression of a gene is caused by an elevated rate of transcription as compared to the native transcription rate for that gene. In other exemplary embodiments, overexpression is caused by an elevated rate of translation of the gene compared to the native translation rate for that gene. Methods of testing for overexpression are well known in the art, for example transcribed RNA levels can be assessed using rtPCR and protein levels can be assessed using SDS page gel analysis.

In other embodiments, the polypeptide, polynucleotide, or gene having an altered level of expression is “attenuated” or has a “decreased level of expression” or is “down-regulated.” As used herein, these terms mean to express or cause to be expressed a polynucleotide, polypeptide, or gene in a cell at a lesser concentration than is normally expressed in a corresponding control cell (e.g., wild type cell) under the same conditions. In other words, the term “attenuate” means to weaken, reduce, or diminish. For example, a polypeptide can be attenuated by modifying the polypeptide to reduce its activity (e.g., by modifying a nucleotide sequence that encodes the polypeptide).

A polynucleotide or polypeptide can be attenuated using any method known in the art. For example, in some exemplary embodiments, the expression of a gene or polypeptide encoded by the gene is attenuated by mutating the regulatory polynucleotide sequences which control expression of the gene. In other exemplary embodiments, the expression of a gene or polypeptide encoded by the gene is attenuated by overexpressing a repressor protein, or by providing an exogenous regulatory element that activates a repressor protein. In still other exemplary embodiments, DNA- or RNA-based gene silencing methods are used to attenuate the expression of a gene or polynucleotide. In some embodiments, the expression of a gene or polypeptide is completely attenuated, e.g., by deleting all or a portion of the polynucleotide sequence of a gene.

The degree of overexpression or attenuation can be 1.5-fold or more, e.g., 2-fold or more, 3-fold or more, 5-fold or more, 10-fold or more, or 15-fold or more. Alternatively, or in addition, the degree of overexpression or attenuation can be 500-fold or less, e.g., 100-fold or less, 50-fold or less, 25-fold or less, or 20-fold or less. Thus, the degree of overexpression or attenuation can be bounded by any two of the above endpoints. For example, the degree of overexpression or attenuation can be 1.5-500-fold, 2-50-fold, 10-25-fold, or 15-20-fold.

As used herein, “substantially free” refers to a condition wherein the recombinant microbe comprises none or almost none of the component it is deemed to be “substantially free” of. For example, the recombinant microbe would be substantially free of the component if it contained less than about 5 wt %, less than about 4 wt %, less than about 3 wt %, less than about 2 wt %, less than about 1 wt %, less than about 0.5 wt %, less than about 0.1 wt %, less than about 0.05 wt %, less than about 0.01 wt %, or about 0 wt % of the component normally found in the microbe. Alternatively, the term “substantially free” can refer to a low amount of the component in relation to another component within the recombinant microbe. For example, a recombinant E. coli is substantially free of polyunsaturated fatty acids or derivatives thereof if the polyunsaturated fatty acids or derivatives thereof comprise about 5 wt % or less of the total amount of fatty acids and derivatives thereof within the E coli. Alternatively, the recombinant E. coli would be considered substantially free of polyunsaturated fatty acids or derivatives thereof if the polyunsaturated fatty acids or derivatives thereof comprise less than about 4 wt %, less than about 3 wt %, less than about 2 wt %, less than about 1 wt %, less than about 0.5 wt %, less than about 0.1 wt %, less than about 0.05 wt %, less than about 0.01 wt %, or about 0 wt % of the total amount of fatty acids and derivatives thereof within the E coli.

As used herein, “modified activity” or an “altered level of activity” of a protein/polypeptide in a recombinant host cell refers to a difference in one or more characteristics in the activity the protein/polypeptide as compared to the characteristics of an appropriate control protein e.g., the corresponding parent protein or corresponding wild type protein. Thus, in exemplary embodiments, a difference in activity of a protein having “modified activity” as compared to a corresponding control protein is determined by measuring the activity of the modified protein in a recombinant host cell and comparing that to a measure of the same activity of a corresponding control protein in an otherwise isogenic host cell. Modified activities can be the result of, for example, changes in the structure of the protein (e.g., changes to the primary structure, such as e.g., changes to the protein's nucleotide coding sequence that result in changes in substrate specificity, changes in observed kinetic parameters, changes in solubility, etc.); changes in protein stability (e.g., increased or decreased degradation of the protein) etc.

The term “recombinant” as used herein, refers to a genetically modified polynucleotide, polypeptide, cell, tissue, or organism. When used with reference to a cell, the term “recombinant” indicates that the cell has been modified by the introduction of a heterologous nucleic acid or protein or has been modified by alteration of a native nucleic acid or protein, or that the cell is derived from a cell so modified and that the derived cell comprises the modification. Thus, for example, “recombinant cells” or equivalently “recombinant host cells” may be modified to express genes that are not found within the native (non-recombinant) form of the cell or may be modified to abnormally express native genes e.g., native genes may be overexpressed, underexpressed or not expressed at all. A recombinant cell can be derived from a microorganism or microbe such as a bacterium (including proteobacterium and cyanobacterium), archaea, a virus, algae, or a fungus. In addition, a recombinant cell can be derived from a plant or an animal cell. In exemplary embodiments, a “recombinant host cell” or “recombinant cell” is used to produce one or more fatty acids or derivatives thereof including, but not limited to, fatty esters, fatty alcohols, fatty alcohol acetates, fatty aldehydes, fatty amines, ω-hydroxy fatty acids, ω-hydroxy fatty esters, ω-carboxy fatty esters, α,ω-fatty diacids, α,ω-fatty diesters, α,ω-fatty diols, etc. Therefore, in some exemplary embodiments a “recombinant host cell” is a “production host” or equivalently, a “production host cell”.

When used with reference to a polynucleotide, the term “recombinant” indicates that the polynucleotide has been modified by comparison to the native or naturally occurring form of the polynucleotide or has been modified by comparison to a naturally occurring variant of the polynucleotide. In an exemplary embodiment, a recombinant polynucleotide (or a copy or complement of a recombinant polynucleotide) is one that has been manipulated by the hand of man to be different from its naturally occurring form. Thus, in an exemplary embodiment, a recombinant polynucleotide is a mutant form of a native gene or a mutant form of a naturally occurring variant of a native gene wherein the mutation is made by intentional human manipulation e.g., made by saturation mutagenesis using mutagenic oligonucleotides, through the use of UV radiation, mutagenic chemicals, chemical synthesis etc. Such a recombinant polynucleotide might comprise one or more point mutations, deletions and/or insertions relative to the native or naturally occurring variant form of the gene. Similarly, a polynucleotide comprising a promoter operably linked to a second polynucleotide (e.g., a coding sequence) is a “recombinant” polynucleotide. Thus, a recombinant polynucleotide comprises polynucleotide combinations that are not found in nature. A recombinant protein (discussed supra) is typically one that is expressed from a recombinant polynucleotide, and recombinant cells, tissues, and organisms are those that comprise recombinant sequences (polynucleotide and/or polypeptide).

The term “vector,” as used herein, refers to a polynucleotide sequence that contains a gene of interest (e.g., it encodes one or more proteins or enzymes described herein) and a promoter operably linked to the fatty acid biosynthetic polynucleotide sequence of interest. Once a polynucleotide sequence(s) encoding a fatty acid biosynthetic pathway polypeptide has been prepared and isolated, various methods may be used to construct expression cassettes, vectors and other DNA constructs. The skilled artisan is well aware of the genetic elements that must be present on an expression construct/vector in order to successfully transform, select, and propagate the expression construct in host cells. Techniques for manipulation of nucleic acids such as subcloning nucleic acid sequences into expression vectors, labeling probes, DNA hybridization are well known in the art.

As used herein, the term “microbe” or “microorganism” refers generally to a microscopic organism. Microbes can be prokaryotic or eukaryotic. Exemplary prokaryotic microbes include e.g., bacteria (including y-proteobacteria), archaea, cyanobacteria, etc. An exemplary proteobacterium is Escherichia coli. Exemplary eukaryotic microorganisms include e.g., yeast, protozoa, algae, etc. In exemplary embodiments, a “recombinant microbe” is a microbe that has been genetically altered and thereby expresses or encompasses an exogenous and/or a heterologous nucleic acid sequence and/or an exogenous and/or a heterologous peptide, polypeptide, or protein.

A microbe as used herein, can grow on a carbon source e.g., a simple carbon source. The recombinant microbe may be a gamma proteobacterium (also known as a y-proteobacterium), a cyanobacterium, a yeast, or an algae.

In some embodiments, the recombinant proteobacterium may be Escherichia coli, Salmonella spp., Vibrio natriegens, Pseudomonas aeruginosa, Pseudomonas putida, Pseudomonas fluorescens, Xanthomonas axonopodis, Pseudomonas syringae, Xyella fastidiosa, Marinobacter aquaeolei, Yersinia pestis, Bacillus spp., Lactobacillus spp., Zymomonas spp., Streptomyces spp., or Vibrio cholerae.

In some embodiments, the recombinant cyanobacterium may be Synechococcus elongatus PCC7942 or Synechocystis sp. PCC6803.

In some embodiments, the recombinant yeast may be Saccharomyces cerevisiae, Scheffersomyces stipitis, Schizosaccharomyces pombe, Kluyveromyces marxianus, K. lactis, Pichia pastoris, Hansenula polymorpha, or Yarrowia lipolytica.

In some embodiments, the recombinant algae may be Botryococcus braunii, Nannochloropsis gaditina, Chlamydomonas reinhardtii, Chlorella vulgaris, Spirulina platensis, Ostreococcus tauri, Phaeodactylum tricornutum, Symbiodinium sp., algal phytoplanktons, Saccharina japonica, Chlorococcum spp., and Spirogyra spp.

As used herein, the term “culture” typically refers to a liquid media comprising viable cells. In one embodiment, a culture comprises cells reproducing in a predetermined culture media under controlled conditions, for example, a culture of recombinant host cells grown in liquid media comprising a selected carbon source and nitrogen.

“Culturing” or “cultivation” refers to growing a population of recombinant host cells (e.g., recombinant microbes) under suitable conditions in a liquid or on a solid medium. In particular embodiments, culturing refers to the fermentative bioconversion of a substrate to an end-product. Culturing media are well-known and individual components of such culture media are available from commercial sources, e.g., under the Difco™ and BBL™ trademarks. In one non-limiting example, the aqueous nutrient medium is a “rich medium” comprising complex sources of nitrogen, salts, and carbon, such as YP medium, comprising 10 g/L of peptone and 10 g/L yeast extract of such a medium.

For small scale production, the host cells engineered to enzymatically produced lactone compositions are typically grown in batches of, for example, about 100 μL, 200 μL, 300 μL, 400 μL, 500 μL, 1 mL, 5 mL, 10 mL, 15 mL, 25 mL, 50 mL, 75 mL, 100 mL, 500 mL, 1 L, 2 L, 5 L, or 10 L.

For large scale production, the engineered host cells can be grown in cultures having a volume batches of about 10 L, 100 L, 1000 L, 10,000 L, 100,000 L, 1,000,000 L or larger; fermented; and induced to express any desired polynucleotide sequence.

A “production host” or equivalently a “production host cell” is a cell used to produce products. As disclosed herein, a production host is typically modified to express or overexpress selected genes, or to have attenuated expression of selected genes. Thus, a production host or a “production host cell” is a recombinant host or equivalently a recombinant host cell. Non-limiting examples of production hosts include, e.g., recombinant microbes as disclosed above. An exemplary production host is a recombinant proteobacterium comprising a heterologous R-3-hydroxy-acyl-CoA dehydratase or a heterologous R-specific enoyl-CoA hydratase, and optionally, a heterologous trans-2-enoyl-CoA reductase. Another exemplary production host is a recombinant proteobacterium comprising a heterologous 3-oxoacyl-CoA reductase, a heterologous β-ketoacyl-CoA-reductase, or heterologous 3-hydroxy acyl-CoA dehydrogenase; a heterologous R-3-hydroxy-acyl-CoA dehydratase or a heterologous R-specific enoyl-CoA hydratase; and optionally, a heterologous trans-2-enoyl-CoA reductase

As used herein, the terms “purify,” “purified,” or “purification” mean the removal or isolation of a molecule from its environment by, for example, isolation or separation. “Substantially purified” molecules are at least about 60% free (e.g., at least about 65% free, at least about 70% free, at least about 75% free, at least about 80% free, at least about 85% free, at least about 90% free, at least about 95% free, at least about 96% free, at least about 97% free, at least about 98% free, at least about 99% free) from other components with which they are associated. As used herein, these terms also refer to the removal of contaminants from a sample.

As used herein, the term “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, cellobiose, and turanose; cellulosic material and variants such as hemicelluloses, methyl cellulose and sodium carboxymethyl cellulose; succinate, lactate, and acetate; alcohols, such as ethanol, methanol, and glycerol, or mixtures thereof. The carbon source can also be a product of photosynthesis, such as glucose. In certain embodiments, the carbon source is a biomass. In other embodiments, the carbon source is glucose. In other embodiments the carbon source is sucrose. In other embodiments the carbon source is glycerol. In other embodiments, the carbon source is a simple carbon source such as e.g., glucose. In other embodiments, the carbon source is a renewable carbon source. In other embodiment, the carbon source is natural gas. In other embodiments the carbon source comprises one or more components of natural gas, such as methane, ethane, or propane. In other embodiments, the carbon source is flu gas or synthesis gas. In still other embodiments, the carbon source comprises one or more components of flu or synthesis gas such as carbon monoxide, carbon dioxide, hydrogen, etc. As used herein, the term “carbon source” or “simple carbon source” specifically excludes oleochemicals such as e.g., saturated or unsaturated fatty acids.

### II. Enzymes

As used herein, the term “R-3-hydroxy-acyl-CoA dehydratase” refers to enzymes that convert 3-hydroxy-acyl-CoA to trans-2-enoyl-CoA. The R-3-hydroxy-acyl-CoA dehydratase may be native to the recombinant cell or microbe (i.e., from or derived from the same species), or it can be heterologous (i.e., from or derived from a different species). The R-3-hydroxy-acyl-CoA dehydratase may be endogenous, wherein the enzyme or a polynucleotide encoding the enzyme (e.g., RNA, DNA, mRNA) is produced by the cell. The R-3-hydroxy-acyl-CoA dehydratase may be heterologous, wherein a polynucleotide, nucleic acid sequence, or gene encoding the enzyme is exogenous and is not produced by the cell, but instead is added to the cell from outside the cell. R-3-hydroxy-acyl-CoA dehydratase may be described by EC 4.2.1.134 or EC 4.2.1.55.

As used herein, the term “R-specific enoyl-CoA hydratase” refers to enzymes that can convert 3-hydroxy-acyl-CoA to trans-2-enoyl-CoA. The R-specific enoyl-CoA hydratase may be native to the recombinant cell or microbe (i.e., from or derived from the same species), or it may be heterologous (i.e., from or derived from a different species). The R-specific enoyl-CoA hydratase may be endogenous, wherein the enzyme or a polynucleotide encoding the enzyme (e.g., RNA, DNA, mRNA) is produced by the cell. Alternatively, the R-specific enoyl-CoA hydratase may be heterologous, wherein a polynucleotide, nucleic acid sequence, or gene encoding the enzyme is exogenous, and is not produced by the cell, but instead is added to the cell from outside the cell. R-specific enoyl-CoA hydratase may be described by the number EC 4.2.1.119 or EC 4.2.1.17, and can also be referred to a an enoyl-CoA hydratase 2, or a 2-enoyl-CoA hydratase 2, or, in some cases, an MaoC family dehydratase.

The R-3-hydroxy-acyl-CoA dehydratase and R-specific enoyl-CoA hydratase are interchangeable in the biochemical synthetic pathway for producing fatty acid derivatives. The recombinant cell or microbe can comprise an endogenous nucleic acid sequence or endogenous gene encoding a native R-3-hydroxy-acyl-CoA dehydratase or R-specific enoyl-CoA hydratase. In other embodiments, a native R-3-hydroxy-acyl-CoA dehydratase or R-specific enoyl-CoA hydratase can be encoded by an exogenous nucleic acid sequence or an exogenous gene, such that the encoding nucleic acid sequence or gene is added to the cell from outside the cell, for example, where overexpression of the native enzyme is desired. Overexpression of a native enzyme, such as an R-3-hydroxy-acyl-CoA dehydratase or an R-specific enoyl-CoA hydratase, can also be achieved by other methods known in the art, such as, for example, by placing the encoding nucleic acid sequence or gene under control of a different (e.g., a more active, or constitutively active, or stronger) promoter, or by modifying the native or endogenous promoter, or by modifying other associated regulatory elements. In such a case, the encoding nucleic acid sequence with the modified or altered regulatory element(s) is considered an exogenous nucleic acid sequence. A native, endogenous, or heterologous R-3-hydroxy-acyl-CoA dehydratase or R-specific enoyl-CoA hydratase can be expressed or overexpressed in the recombinant cell or microbe. In some embodiments, the R-3-hydroxy-acyl-CoA dehydratase or R-specific enoyl-CoA hydratase is native to the cell and is overexpressed. In other embodiments, the R-3-hydroxy-acyl-CoA dehydratase or R-specific enoyl-CoA hydratase is heterologous to the cell and is expressed in the cell. In certain embodiments, the R-3-hydroxy-acyl-CoA dehydratase or R-specific enoyl-CoA hydratase is any one of those listed in Table 1 below, or comprises the amino acid sequence set forth in any one of SEQ ID NOs: 1-13, or is a homolog of any of the enzymes listed in Table 1 or a homolog of any one of SEQ ID NOs: 1-13, having at least about 30%, 35%, 40%, 45%, 50%, 55%, 60%, 65%, 70%, 75%, 80%, 85%, 90%, 95% or more, sequence identity thereto.

Examples of R-3-hydroxy-acyl-CoA dehydratases or R-specific enoyl-CoA hydratases that can be used in the pathways (or bypass pathways) or methods herein, or that can be expressed or overexpressed in the recombinant cells, microbes, or microorganisms provided herein, are shown in Table 1 below.

TABLE 1
Examples of R-3-hydroxy-acyl-CoA dehydratases or R-specific enoyl-CoA hydratases

| Name/Designation | Microorganism | Accession Number | SEQ ID NO: |
|---|---|---|---|
| PhaJ1 (PhaJ1_P.put) | Pseudomonas putida | UniProtKB Q88EB4 or | 1 |
| | | Q8KRE2, GenBank | |
| | | WP_003254624 | |
| PhaJ4 (PhaJ4_P.put) | Pseudomonas putida | UniProtKB Q88DK8 or | 2 |
| | | E9P3X9, GenBank | |
| | | WP_010955428 | |
| PhaJ3 (PhaJ3_P.aer) | Pseudomonas | UniProtKB Q9HV20 or | 3 |
| | aeruginosa | G3XCK1, GenBank | |
| | | WP_003095272 | |
| PhaJ4 (PhaJ4_P.aer) | Pseudomonas | UniProt KB Q9HX12 or | 4 |
| | aeruginosa | G3XCK8, GenBank | |
| | | WP_003093220 | |
| MaoC Family | Pseudomonas | GenBank WP_074981696 | 5 |
| Dehydratase | citronellolis | | |
| MaoC Family | Pseudomonas | GenBank WP_106738883 | 6 |
| Dehydratase | mendocina | | |
| MaoC9 (MaoC | Pseudomonas putida | GenBank AAN66207.1 | 7 |
| Doman Protein) | | | |
| MaoC/Dehydratase | Pseudomonas | GenBank AJG15930.1 | 8 |
| | plecoglossicida | | |
| MaoC Family | Pseudomonas | GenBank WP_108481500.1 | 9 |
| Dehydratase | plecoglossicida | | |
| PhaJ4 | Pseudomonas mosselii | GenBank AJZ69596.1 | 10 |
| MaoC Family | Pseudomonas | GenBank WP_016393459.1 | 11 |
| Dehydratase | plecoglossicida | | |
| MaoC Family | Pseudomonas fulva | GenBank WP_028687162.1 | 12 |
| Dehydratase | | | |
| MaoC Family | Pseudomonas fulva | GenBank WP_182140159.1 | 13 |
| Dehydratase | | | |

As used herein, the term “trans-2-enoyl-CoA reductase” refers to an enzyme that reduces or converts trans-2-enoyl-CoA to the corresponding, fully reduced acyl-CoA. When NADH or NADPH is a cofactor of the reaction, the reduction is irreversible. The trans-2-enoyl-CoA reductase may be native to the recombinant cell or microbe (i.e., from or derived from the same species), or it may be heterologous (i.e., from or derived from a different species). The trans-2-enoyl-CoA reductase may be endogenous, wherein the enzyme, or a polynucleotide encoding the enzyme (e.g., RNA, DNA, mRNA) is produced by the cell. Alternatively, the trans-2-enoyl-CoA reductase may be heterologous, wherein a polynucleotide, nucleic acid sequence, or gene encoding the enzyme is exogenous, and is not produced by the cell, but instead is added to the cell from outside the cell. In some embodiments, the trans-2-enoyl-CoA reductase described herein may belong to EC 1.3.1.44. In some embodiments, the trans-2-enoy-CoA reductase can be referred to as TER and/or FabV.

The recombinant cell or microbe can comprise an endogenous nucleic acid sequence or endogenous gene encoding a native trans-2-enoyl-CoA reductase. In other embodiments, a native trans-2-enoyl-CoA reductase can be encoded by an exogenous nucleic acid sequence or an exogenous gene, such that the encoding nucleic acid sequence or gene is added to the cell from outside the cell, for example, where overexpression of the native enzyme is desired. Overexpression of a native enzyme, such as a trans-2-enoyl-CoA reductase, can also be achieved by other methods known in the art, such as, for example, by placing the encoding nucleic acid sequence or gene under control of a different (e.g., a more active, or constitutively active, or stronger) promoter, or by modifying the native or endogenous promoter, or by modifying other associated regulatory elements. In such a case, the encoding nucleic acid sequence with the modified or altered regulatory element(s) is considered an exogenous nucleic acid sequence. A native, endogenous, or heterologous trans-2-enoyl-CoA reductase can be expressed or overexpressed in the recombinant cell or microbe. In some embodiments, the trans-2-enoyl-CoA reductase is native to the cell and is overexpressed. In other embodiments, the trans-2-enoyl-CoA reductase is heterologous to the cell and is expressed in the cell. In certain embodiments, the trans-2-enoyl-CoA reductase is any one of those listed in Table 2 below, or comprises the amino acid sequence set forth in any one of SEQ ID NOs: 14-25, or is a homolog of any of the enzymes listed in Table 2 or a homolog of any one of SEQ ID NOs: 14-25, having at least about 30%, 35%, 40%, 45%, 50%, 55%, 60%, 65%, 70%, 75%, 80%, 85%, 90%, 95% or more, sequence identity thereto.

Examples of trans-2-enoyl-CoA reductases (with source microbes), that can be used in the pathways (e.g., bypass pathways) or methods herein, or that can be expressed or overexpressed in the recombinant cells, microbes, or microorganisms provided herein, are shown in Table 2 below.

TABLE 2
Examples of trans-2-enoyl-CoA reductases

| Name/Designation | Microorganism | Accession Number | SEQ ID NO: |
|---|---|---|---|
| FabV or TER | Treponema denticola | UniProtKB Q73Q47 | 14 |
| (TER_T.den) | | | |
| TER | Euglena gracilis | UniProtKB Q5EU90 | 15 |
| TER | Enterovibrio coralii | WP_067414352 (GenBank) | 16 |
| TER | Lactobacillus | WP_057889432 (GenBank) | 17 |
| | oligofermentans | | |
| TER or FabV | Paucilactobacillus | KRL57745 | 18 |
| | oligofermentans | (GenBank) | |
| TER | Vibrio spp. | WP_009705827 (GenBank) | 19 |
| TER | Treponema | WP_021686193 (GenBank) | 20 |
| | lecithinolyticum | | |
| TER or FabV | Treponema pedis | WP_020965800 (GenBank) | 21 |
| TER or FabV | Treponema vincentii | WP_162662171 (GenBank) | 22 |
| TER or FabV | Treponema sp. | GenBank NLK45212; | 23 |
| | | UniProtKB A0A7X8IEX0 | |
| TER or Fabv | Treponema | WP_013759269 (GenBank) | 24 |
| | brennaborense | | |
| TER or FabV | Treponema parvum | WP_210121374 (GenBank) | 25 |

As used herein, a 3-oxoacyl-CoA reductase, β-ketoacyl-CoA-reductase, or 3-hydroxy acyl-CoA dehydrogenase (or β-hydroxy acyl-CoA dehydrogenase) refers to an enzyme that can convert or reduce a 3-oxo-acyl-CoA to the corresponding 3-hydroxy-acyl-CoA. The 3-oxoacyl-CoA reductase, β-ketoacyl-CoA-reductase, or 3-hydroxy acyl-CoA dehydrogenase may be native to the recombinant cell or microbe (i.e., from the same species), or it may be heterologous (i.e., from or derived from a different species). The 3-oxoacyl-CoA reductase, β-ketoacyl-CoA-reductase, or 3-hydroxy acyl-CoA dehydrogenase may be endogenous, wherein the enzyme, or a polynucleotide encoding the enzyme (e.g., RNA, DNA, mRNA) is produced by the cell. Alternatively, the 3-oxoacyl-CoA reductase, β-ketoacyl-CoA-reductase, or 3-hydroxy acyl-CoA dehydrogenase may be heterologous, wherein a polynucleotide, nucleic acid sequence, or gene encoding the enzyme is exogenous, and is not produced by the cell, but instead is added to the cell from outside the cell. In some embodiments, the 3-oxoacyl-CoA reductase, β-ketoacyl-CoA-reductase, or 3-hydroxy acyl-CoA dehydrogenase can be classified under EC 1.1.1.35, 1.1.1.36, or 1.1.1.157, and can also be referred to as a β-hydroxy acyl-CoA dehydrogenase, a beta-hydroxyacyl dehydrogenase, a beta-keto reductase, or an acetoacetyl-CoA reductase.

The recombinant cell or microbe can comprise an endogenous nucleic acid sequence or endogenous gene encoding a native 3-oxoacyl-CoA reductase, β-ketoacyl-CoA-reductase, or 3-hydroxy acyl-CoA dehydrogenase. In other embodiments, a native 3-oxoacyl-CoA reductase, β-ketoacyl-CoA-reductase, or 3-hydroxy acyl-CoA dehydrogenase can be encoded by an exogenous nucleic acid sequence or an exogenous gene, such that the encoding nucleic acid sequence or gene is added to the cell from outside the cell, for example, where overexpression of the native enzyme is desired. Overexpression of a native enzyme, such as a 3-oxoacyl-CoA reductase, β-ketoacyl-CoA-reductase, or 3-hydroxy acyl-CoA dehydrogenase, can also be achieved by other methods known in the art, such as, for example, by placing the encoding nucleic acid sequence or gene under control of a different (e.g., a more active, or constitutively active, or stronger) promoter, or by modifying the native or endogenous promoter, or by modifying other associated regulatory elements. In such a case, the encoding nucleic acid sequence with the modified or altered regulatory element(s) is considered an exogenous nucleic acid sequence. A native, endogenous, or heterologous 3-oxoacyl-CoA reductase, β-ketoacyl-CoA-reductase, or 3-hydroxy acyl-CoA dehydrogenase can be expressed or overexpressed in the recombinant cell or microbe. In some embodiments, the 3-oxoacyl-CoA reductase, β-ketoacyl-CoA-reductase, or 3-hydroxy acyl-CoA dehydrogenase is native to the cell and is overexpressed. In other embodiments, the 3-oxoacyl-CoA reductase, β-ketoacyl-CoA-reductase, or 3-hydroxy acyl-CoA dehydrogenase is heterologous to the cell and is expressed in the cell. In certain embodiments, the 3-oxoacyl-CoA reductase, β-ketoacyl-CoA-reductase, or 3-hydroxy acyl-CoA dehydrogenase is any one of those listed in Table 2A below, or comprises the amino acid sequence set forth in any one of SEQ ID NOs: 38-45, or is a homolog of any of the enzymes listed in Table 2A or a homolog of any one of SEQ ID NOs: 38-45, having at least about 30%, 35%, 40%, 45%, 50%, 55%, 60%, 65%, 70%, 75%, 80%, 85%, 90%, 95% or more, sequence identity thereto.

Examples of 3-oxoacyl-CoA reductases, β-ketoacyl-CoA-reductases, or 3-hydroxy acyl-CoA dehydrogenases (with source microbes), that can be used in the pathways (e.g., bypass pathways) or methods herein, or that can be expressed or overexpressed in the recombinant cells, microbes, or microorganisms provided herein, are shown in Table 2A below.

TABLE 2A
Examples of 3-oxoacyl-CoA reductases, β-ketoacyl-
CoA-reductases, or 3-hydroxy acyl-CoA dehydrogenases

| Enzyme and/or Gene | | GenBank Accession | |
|---|---|---|---|
| symbol | Organism | Number | SEQ ID NO. |
| acetoacetyl-CoA reductase/ | Cupriavidus necator | P14697 | 38 |
| phaB | (Ralstonia eutropha) | | |
| 3-hydroxybutyryl-CoA | Cupriavidus necator | SCU73809 | 39 |
| dehydrogenase/hbd | (Ralstonia eutropha) | | |
| 3-hydroxyacyl-CoA | Cupriavidus necator | CAJ91433 | 40 |
| dehydrogenase/paaH1 | H16 (Ralstonia | | |
| | eutropha) | | |
| Putative 3-oxoacyl-[acyl- | Cupriavidus | B3R6T4 | 41 |
| carrier-protein] reductase | taiwanensis | | |
| 3-oxoacyl-ACP reductase | Bacillus sp. SG-1 | WP_006837618 | 42 |
| (FabG)/fabG | | | |
| 3-ketoacyl-ACP reductase | Burkholderia | WP_059872892 | 43 |
| | multivorans | | |
| acetoacetyl-CoA reductase/ | Pseudomonas putida | BAB96554 | 44 |
| phbB | | | |
| 3-hydroxybutyryl-CoA | Megasphaera elsdenii | CCC73669 | 45 |
| dehydrogenase/hbd | | | |
| (MELS_1448) | | | |

As used herein, the term “acyl-CoA thioesterase” refers to an enzyme that converts acyl-CoA or trans-2-enoyl-CoA to the corresponding fatty acid or trans-2 fatty acid, respectively. The acyl-CoA thioesterase may be native to the recombinant cell or microbe (i.e., from or derived from the same species), or it may be heterologous (i.e., from or derived from a different species). The acyl-CoA thioesterase may be endogenous, wherein the enzyme or a polynucleotide encoding the enzyme (e.g., RNA, DNA, mRNA) is produced by the cell. Alternatively, the acyl-CoA thioesterase may be heterologous, wherein a polynucleotide, nucleic acid sequence, or gene encoding the enzyme is not produced by the cell, but instead is added to the cell from outside the cell. The acyl-CoA thioesterase may be described by the number EC 3.1.2.2 or EC 3.1.2.20, and can also be referred to as a fatty-acyl-CoA hydrolase, a long-chain fatty-acyl-CoA hydrolase, or an acyl-CoA hydrolase.

As used here, the term “acyl-ACP thioesterase” refers to an enzyme that converts acyl-ACP or 3-hydroxy acyl-ACP or 3-keto-acyl-ACP (B-keto-acyl-ACP) to the corresponding fatty acid or 3-hydroxy fatty acid or 3-oxo fatty acid, respectively. The acyl-ACP thioesterase may be native to the recombinant, cell, microorganism, or microbe, or may be heterologous. The acyl-ACP thioesterase may be endogenous, wherein the enzyme or a polynucleotide encoding the enzyme (e.g., RNA, DNA, mRNA) is produced by the cell. Alternatively, the acyl-ACP thioesterase may be heterologous, wherein a polynucleotide, nucleic acid sequence, or gene encoding the enzyme is not produced by the cell, but instead is added to the cell from outside the cell. The acyl-ACP thioesterase may be described by the number EC 3.1.2.14 or EC 3.1.2.21, and can also be referred to as an acyl-ACP hydrolase.

As used herein, the term “acyl-CoA synthetase” (alternatively “acyl-CoA synthase” or “acyl-CoA ligase”) refers to an enzyme that can reactivate or convert free fatty acids to the corresponding acyl-CoAs, or free 3-hydroxy fatty acids to the corresponding 3-hydroxy-acyl-CoAs, or free 3-oxo fatty acids to the corresponding 3-oxo acyl-CoAs. The acyl-CoA synthetase may be native to the recombinant cell, microorganism, or microbe, or may be heterologous. The acyl-CoA synthetase may be endogenous, wherein the enzyme or a polynucleotide encoding the enzyme (e.g., RNA, DNA, mRNA) is produced by the cell. Alternatively, the acyl-CoA synthetase may be heterologous, wherein a polynucleotide, nucleic acid, or gene encoding the enzyme is not produced by the cell, but instead is added to the cell from outside the cell. Acyl-CoA synthetase may be described by the number EC 6.2.1.3, and can also be referred to as a long-chain-fatty acid CoA ligase, or an acyl-CoA ligase.

As used here, the term “ester synthase” refers to an enzyme that esterifies or converts an acyl-CoA, in the presence of an alcohol (e.g., methanol or ethanol) to the corresponding fatty acid ester. The ester synthase may be native to the recombinant cell or microbe, or may be heterologous. The ester synthase may be endogenous, wherein the enzyme or a polynucleotide encoding the enzyme (e.g., RNA, DNA, mRNA) is produced by the cell. Alternatively, the ester synthase may be heterologous, wherein a polynucleotide, nucleic acid or gene encoding the enzyme is not produced by the cell, but instead is added to the cell from outside the cell. The ester synthase may be described by the number EC 2.3.1.20.

As used herein, the term “β-ketoacyl-ACP-synthase,” which includes-ketoacyl-ACP synthase I, e.g., “FabB” and/or β-ketoacyl-ACP synthase II, e.g., “FabF,” refers to enzymes that catalyze the condensation reactions to elongate the fatty acid chain. The β-ketoacyl-ACP synthase may be native to the recombinant cell or microbe or may be heterologous. The β-ketoacyl-ACP-synthase may be endogenous, wherein the enzyme or a polynucleotide encoding the enzyme (e.g., RNA, DNA, mRNA) is produced by the cell. Alternatively, the β-ketoacyl-ACP-synthase may be heterologous, wherein a polynucleotide, nucleic acid, or gene encoding the enzyme is not produced by the cell, but instead is added to the cell from outside the cell. β-ketoacyl-ACP-synthase may be described by the number EC 2.3.1.41 (β-ketoacyl-ACP-synthase I; e.g., FabB), or EC 2.3.1.179 (β-ketoacyl-ACP-synthase II; e.g., FabF).

As used herein, the term “alcohol dehydrogenase” refers to an enzyme that catalyzes the interconversion between aliphatic alcohols (e.g., aliphatic medium-chain alcohols) and their corresponding aldehydes. In some embodiments, and under some conditions, the alcohol dehydrogenase converts an alcohol into an aldehyde. In some embodiments and under some conditions, the alcohol dehydrogenase converts an aldehyde into an alcohol. The alcohol dehydrogenase may be native to the recombinant cell or microbe or may be heterologous. The alcohol dehydrogenase may be endogenous, wherein the enzyme, or a polynucleotide encoding the enzyme (e.g., RNA, DNA, mRNA), is produced by the cell. Alternatively, the alcohol dehydrogenase may be heterologous, wherein a polynucleotide, nucleic acid sequence, or gene, encoding the enzyme is not produced by the cell, but instead is added to the cell from outside the cell. The alcohol dehydrogenase may belong to EC 1.1.1.1 or EC 1.1.1.2, or EC 1.1.1.-, and can also be referred to as an aldehyde reductase.

As used herein, the term “alcohol-O-acetyl-transferase” (also known as “alcohol-O-acetyltransferase”) refers to an enzyme that catalyzes the interconversion between acetyl-CoA and an alcohol, and CoA and an acetyl ester. The alcohol-O-acetyl-transferase may be native to the recombinant cell or microbe or may be heterologous. The alcohol-O-acetyl-transferase may be endogenous, wherein the enzyme, or a polynucleotide encoding the enzyme (e.g., RNA, DNA, mRNA), is produced by the cell. Alternatively, the alcohol-O-acetyl-transferase may be heterologous, wherein a polynucleotide, nucleic acid sequence, or gene, encoding the enzyme is not produced by the cell, but instead is added to the cell from outside the cell. The alcohol-O-acetyl-transferase may belong to EC 2.3.1.84.

As used herein, the term “carboxylic acid reductase” refers to an enzyme that converts a fatty acid to its corresponding fatty aldehyde. The carboxylic acid reductase may be native to the recombinant cell or microbe or may be heterologous. The carboxylic acid reductase may be endogenous, wherein the enzyme, or a polynucleotide encoding the enzyme (e.g., RNA, DNA, mRNA), is produced by the cell. Alternatively, the carboxylic acid reductase may be heterologous, wherein a polynucleotide, nucleic acid sequence, or gene, encoding the enzyme is not produced by the cell, but instead is added to the cell from outside the cell. The carboxylic acid reductase described herein may belong to EC 1.2.1.30, and can also be referred to as a carboxylate reductase.

As used herein, the term “acyl-CoA reductase” refers to to an enzyme that converts a fatty acyl-CoA to its corresponding fatty aldehyde. The acyl-CoA reductase may be native to the recombinant cell or microbe or may be heterologous. In some embodiments, the acyl-CoA reductase may be endogenous, wherein the enzyme or a polynucleotide encoding the enzyme (e.g., RNA, DNA, mRNA) is produced by the cell. In another embodiment, the acyl-CoA reductase may be heterologous, wherein a polynucleotide, nucleic acid sequence, or gene, encoding the enzyme is not produced by the cell, but instead is added to the cell from outside the cell. Acyl-CoA reductase may be described by the number EC 1.2.1.50.

As used herein, the term “fatty alcohol forming acyl-CoA reductase” refers to an enzyme or polypeptide that catalyzes the reduction of fatty acyl-CoAs to fatty aldehydes, and that catalyzes the subsequent reduction of the fatty aldehydes to fatty alcohols. The fatty alcohol forming acyl-CoA reductase may be native to the recombinant cell or microbe, i.e., from or derived from the same species as the recombinant cell or microbe, or may be heterologous, i.e., from or derived from an organism or species that is different from the recombinant cell or microbe. The native or heterologous fatty alcohol forming acyl-CoA reductase can be expressed, or can be overexpressed, in the recombinant cell or microbe. In some embodiments, the heterologous native fatty alcohol forming acyl-CoA reductase (FAR) may be endogenous, wherein the enzyme or a polynucleotide encoding the enzyme (e.g., RNA, mRNA, or DNA) is produced by the cell. In another embodiment, the fatty alcohol forming acyl-CoA reductase is heterologous (to the recombinant cell or microbe), and the polynucleotide, nucleic acid sequence, or gene, encoding the enzyme, is exogenous and is not produced by the cell, but instead is added to the cell from outside the cell. Fatty alcohol forming acyl-CoA reductase may be described by EC 1.2.1.84 and can be alternatively referred to as alcohol-forming fatty acyl-CoA reductase. In some embodiments, the fatty alcohol forming acyl-CoA reductase is native to the cell and is overexpressed. In other embodiments, the fatty alcohol forming acyl-CoA reductase is heterologous to the cell and is expressed in the cell.

As used herein, the term “aldehyde dehydrogenase” refers to enzymes that convert aldehydes to carboxylic acids. The aldehyde dehydrogenase may be native to the recombinant cell or microbe, i.e., from or derived from the same species as the recombinant cell or microbe, or may be heterologous, i.e., from or derived from an organism or species that is different from the recombinant cell or microbe. The native or heterologous aldehyde dehydrogenase can be expressed, or can be overexpressed, in the recombinant cell or microbe.

In some embodiments, the native aldehyde dehydrogenase may be endogenous, wherein the enzyme, or a polynucleotide encoding the enzyme (e.g., RNA, DNA, mRNA) is produced by the cell. For example, the recombinant cell or microbe can comprise an endogenous nucleic acid sequence or endogenous gene encoding the native aldehyde dehydrogenase.

In other embodiments, the native aldehyde dehydrogenase can be encoded by an exogenous nucleic acid sequence or an exogenous gene, such that the encoding nucleic acid sequence or gene is added to the cell from outside the cell, for example, where overexpression of the native enzyme is desired. In another embodiment, the aldehyde dehydrogenase is heterologous (to the recombinant cell or microbe), and the polynucleotide, nucleic acid sequence, or gene, encoding the enzyme, is exogenous and is not produced by the cell, but instead is added to the cell from outside the cell. Aldehyde dehydrogenases may be described by EC 1.2.1.3. In some embodiments, the aldehyde dehydrogenase is native to the cell and is overexpressed. In other embodiments, the aldehyde dehydrogenase is heterologous to the cell and is expressed in the cell.

As used herein, the term “ω-hydroxylase” or “omega-hydroxylase” refers to an enzyme or polypeptide that hydroxylates a fatty acid or fatty acid derivative in the ω-position (omega-position), i.e., adds a hydroxy (—OH) group to the ω-position of the fatty acid or derivative thereof. The omega-(@)-position indicates the reduced end of a fatty acid derivative, or the position of the last carbon along the fatty acid derivative chain (farthest from the carboxyl group, for example). The ω-hydroxylase may be native to the recombinant cell or microbe i.e., from or derived from the same species as the recombinant cell or microbe, or may be heterologous, i.e., from or derived from an organism or species that is different from the recombinant cell or microbe. The native or heterologous ω-hydroxylase synthase can be expressed, or can be overexpressed, in the recombinant cell or microbe. In some embodiments, the native ω-hydroxylase may be endogenous, wherein the enzyme, or a polynucleotide encoding the enzyme (e.g., RNA, DNA, mRNA) is produced by the cell. For example, the recombinant cell or microbe can comprise an endogenous nucleic acid sequence or endogenous gene encoding the native ω-hydroxylase. In another embodiment, the ω-hydroxylase is heterologous (to the recombinant cell or microbe), and the polynucleotide, nucleic acid sequence, or gene, encoding the enzyme, is exogenous and is not produced by the cell, but instead is added to the cell from outside the cell. In some embodiments, the ω-hydroxylase may belong to EC 1.14.15.3 or 1.14.14.80, and can alternatively be referred to as a monooxygenase, an alkane 1-monooxygenase, an alkane 1-hydroxylase, a fatty acid omega-hydroxylase, or a long chain fatty acid omega-monooxygenase.

As used herein, “FadR” refers to a transcriptional regulator of fatty acid degradation. FadR inhibits and/or represses transcription of genes required for fatty acid transport and β-oxidation.

In any of the embodiments described herein, any one or more of the fatty acid biosynthesis enzymes and/or fatty acid derivative enzymes described herein, can be native or heterologous to the recombinant cell or microbe (or microorganism). For example, a native enzyme or polypeptide is from or derived from the same species as the recombinant cell or microbe. A heterologous enzyme or polypeptide is from or derived from an organism or species that is different from the recombinant cell or microbe. Any of the native or heterologous enzymes or polypeptides described herein can be expressed, or can be overexpressed, in the recombinant cell or microbe.

The native enzyme or polypeptide, or the encoding polynucleotide sequence or gene, can be endogenous, i.e., found in and produced within the cell. For example, the recombinant cell or microbe or microorganism can comprise an endogenous nucleic acid sequence or endogenous gene encoding the native enzyme or polypeptide. In other embodiments, the native enzyme or polypeptide can be encoded by an exogenous nucleic acid sequence or an exogenous gene, such that the encoding nucleic acid sequence or gene is added to the cell from outside the cell, for example, where overexpression of the native enzyme or polypeptide is desired. Overexpression of a native enzyme or polypeptide, such as any described herein, can also be achieved by other methods known in the art, such as, for example, by placing the encoding nucleic acid sequence or gene under control of a different (e.g., a more active, or constitutively active, or stronger) promoter, or by modifying the native or endogenous promoter, or by modifying other associated regulatory elements. In such a case, the encoding nucleic acid sequence with the modified or altered regulatory element(s) is considered an exogenous nucleic acid sequence.

The gene or nucleic acid sequence encoding a native enzyme or polypeptide can be a non-native variant, for example, where the gene or nucleic acid sequence is operably linked to a non-native regulatory element; in such a case, the non-native gene or nucleic acid sequence typically is referred to herein as an exogenous gene or nucleic acid sequence, even though it can encode a native polypeptide or enzyme.

In other embodiments, any of the enzymes or polypeptides described herein can be a heterologous enzyme or polypeptide, and the polynucleotide, nucleic acid sequence, or gene, encoding the enzyme, is exogenous and is not produced by the cell, but instead is added to the cell from outside the cell.

A native, endogenous, or heterologous enzyme or polypeptide can be expressed or overexpressed in the recombinant cell or microbe or microorganism. For example, in some embodiments, an enzyme or polypeptide is native and is expressed in the recombinant cell or microbe by an endogenous nucleic acid sequence or gene. In other embodiments, the polypeptide or enzyme is native to the cell and is overexpressed, for example, where the recombinant cell or microbe contains an exogenous nucleic acid sequence encoding the native enzyme or polypeptide. In other embodiments, the enzyme or polypeptide is heterologous to the recombinant cell or microbe, and can be expressed or overexpressed in the recombinant cell or microbe by an exogenous nucleic acid sequence.

### III. Novel Biochemical/Biosynthetic (“Bypass”) Pathways, and Recombinant Cells, Microorganism, or Microbes, Comprising Novel Biochemical or Biosynthetic Pathways

As discussed above, byproducts produced by biosynthetic pathways (e.g., acyl-ACP dependent fatty acid biosynthesis pathways) during the production of fatty acids and fatty acid derivatives can reduce the amount (e.g., titer, yield, %, weight %, and/or productivity) of the target compound and the efficiency of the method. The unwanted byproducts generally are derived from or generated from one or more intermediates of an acyl-ACP dependent fatty acid biosynthetic pathway. The unwanted byproducts can be, for example, fatty acids or derivatives thereof containing a 3-hydroxy (3-OH) group, or fatty acids or derivatives thereof containing a 3-oxo (or beta-keto) group, or a combination thereof. It is desirable to avoid (e.g., eliminate or substantially eliminate) or to reduce the production of such byproducts.

Thus, novel biosynthetic pathways, engineered (or modified) to preferentially produce fatty acids and fatty acid derivatives, such as, but not limited to, for example, fatty esters, fatty alcohols, and/or fatty alcohol acetates, and to reduce or eliminate or substantially eliminate the production of byproducts, such as 3-hydroxy fatty acid and 3-hydroxy fatty acid derivative byproducts (e.g., 3-hydroxy fatty esters, 1,3-fatty diols, and/or fatty alcohol 1,3-diacetates) and/or 3-oxo fatty acids and 3-oxo fatty acid derivatives (e.g., 3-oxo fatty esters, 3-oxo fatty alcohols, 3-oxo fatty alcohol acetate esters), are provided herein. Such engineered pathways can be referred to herein as “bypass” pathways. Also provided herein are recombinant cells or microbes that comprise such pathways, as well as methods of using the pathways and the recombinant cells or microbes for the production of compositions comprising fatty acids and derivatives thereof, wherein the compositions comprise a reduced amount of byproducts or are free or substantially free of byproducts.

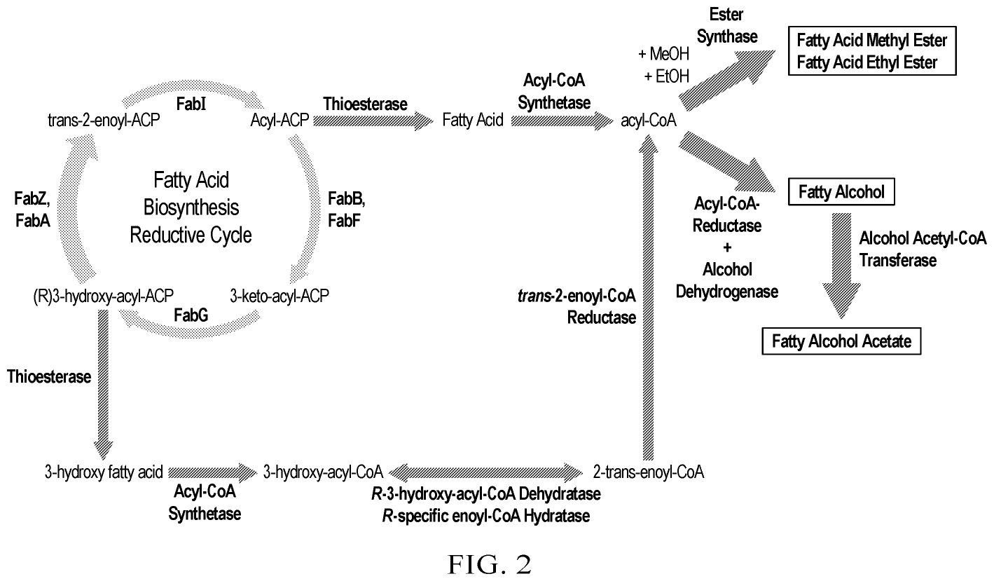

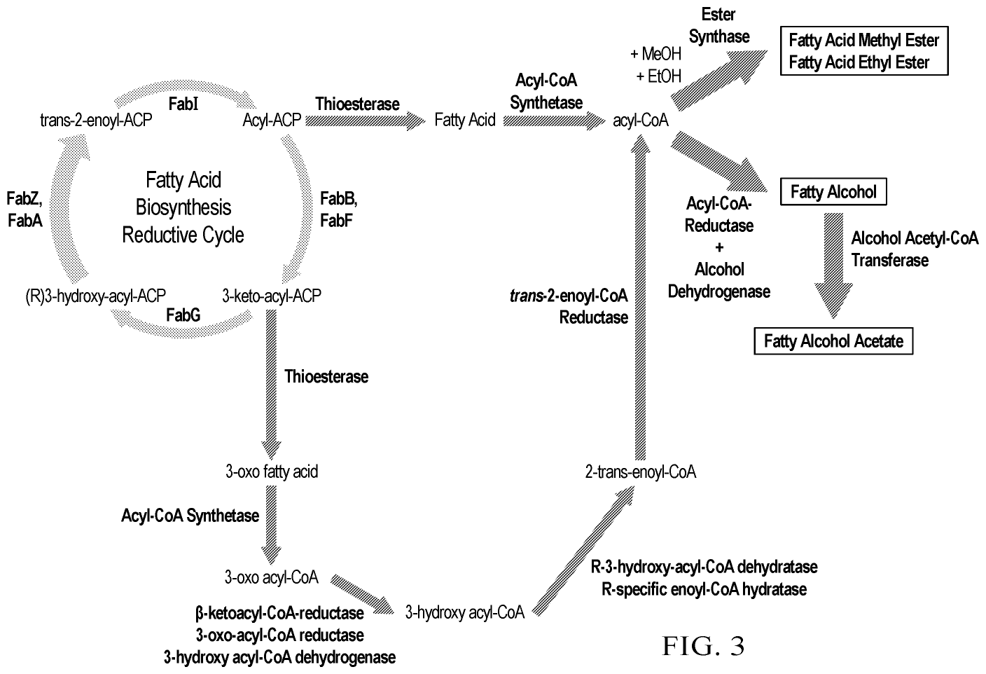

Examples of these novel biochemical synthetic pathways are depicted in FIG. 2 and FIG. 3. In some embodiments, the presence of heterologous a R-3-hydroxy-acyl-CoA dehydratase or a heterologous R-specific enoyl-CoA hydratase, optionally in combination with a heterologous trans-2-enoyl-CoA reductase, drives the production of fatty acids and fatty acid derivatives, such as fatty esters, fatty alcohols, and/or fatty alcohol acetates, while reducing or eliminating or substantially eliminating the production of 3-OH group containing fatty acids and derivatives thereof (i.e., byproducts) (see, e.g., FIG. 2). In other embodiments, the presence of one of a heterologous 3-oxoacyl-CoA reductase, a heterologous β-ketoacyl-CoA-reductase, or a heterologous 3-hydroxy acyl-CoA dehydrogenase; one of a heterologous R-3-hydroxy acyl-CoA dehydratase or a heterologous R-specific enoyl-CoA hydratase; and optionally, a heterologous trans-2-enoyl-CoA reductase drives the production of fatty acids and fatty acid derivatives, such as fatty esters, fatty alcohols, and/or fatty alcohol acetates, while reducing or eliminating or substantially eliminating the production of 3-oxo group containing fatty acids and derivatives thereof (i.e., byproducts) (see, e.g., FIG. 3).

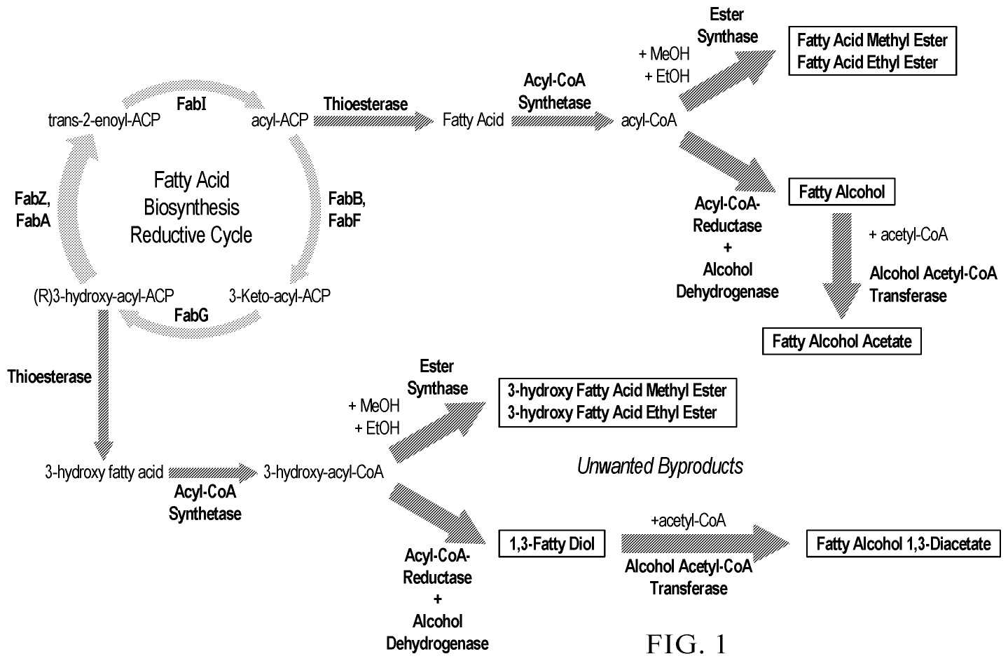

FIG. 1 depicts an acyl-ACP-dependent fatty acid biosynthetic pathway which leads to the generation of undesired products (or byproducts), such as 3-hydroxy containing fatty acids and derivatives thereof, including, but not limited to, for example, 3-hydroxy fatty esters (e.g., 3-hydroxy fatty acid methyl esters, 3-hydroxy fatty acid ethyl esters), 1,3-fatty diols, and/or fatty alcohol 1,3-diacetates. As shown in FIG. 1, and described elsewhere herein, an acyl-ACP thioesterase can convert R-3-hydroxy-acyl-ACPs (produced in the fatty acid biosynthesis reductive cycle) to the corresponding 3-hydroxy fatty acids, which can be converted to the corresponding 3-hydoxy-acyl-CoAs by an acyl-CoA synthetase. The 3-hydroxy-acyl-CoA can then be converted to a 3-hydroxy fatty ester (e.g., 3-hydroxy fatty acid methyl ester or 3-hydroxy fatty acid ethyl ester), a 1,3-fatty diol, and/or a fatty alcohol 1,3-diacetate, depending on which enzymes (fatty acid derivative enzymes) are present or expressed in the cell or microbe.

However, as shown in FIG. 2 (and described elsewhere herein), expression of heterologous R-3-hydroxy-acyl-CoA dehydratase or a heterologous R-specific enoyl-CoA hydratase, optionally in combination with a heterologous trans-2-enoyl-CoA reductase, can drive or increase the production of the non-3-hydroxy fatty acid derivatives (i.e., the fatty acids and derivatives thereof that do not contain a 3-hydroxy group, or the “target products”). The production of 3-hydroxy fatty acids and derivatives thereof (or 3-hydroxy group containing fatty acids and derivatives thereof), i.e., the “byproducts” is also reduced, eliminated, or substantially eliminated. The 3-hydroxy-acyl-CoA can be converted to 2-trans-enoyl-CoA (alternatively termed trans-2-enoyl-CoA) by either the R-3-hydroxy-acyl-CoA dehydratase (EC 4.2.1.134 or EC 4.2.1.55) or R-specific enoyl-CoA hydratase (EC 4.2.1.119 or EC 4.2.1.17). The 2-trans-enoyl-CoA is then reduced to the corresponding acyl-CoA by the trans-2-enoyl-CoA reductase (EC 1.3.1.44), and the acyl-CoA can then be converted to a fatty acid or a fatty acid derivative by the appropriate fatty acid derivative enzyme(s). For example, the acyl-CoA can be converted to: A) a fatty acid alkyl ester by an ester synthase (in the presence of an alcohol); B) a fatty alcohol by an acyl-CoA reductase and an alcohol dehydrogenase, or by a fatty-alcohol-forming acyl-CoA reductase; or C) a fatty alcohol acetate by the additional presence of an alcohol acetyl-CoA transferase (in the presence of acetyl-CoA) (in addition to the enzymes required for the production of a fatty alcohol). Other fatty acid derivatives can also be produced from the acyl-CoAs, as described elsewhere and as known in the art. For example, an acyl-CoA thioesterase can convert the acyl-CoA to the corresponding free fatty acid or an acyl-CoA reductase can convert the acyl-CoA to the corresponding fatty aldehyde. In some embodiments, an acyl-CoA thioesterase can convert the acyl-CoA to the corresponding free fatty acid, which can then be converted to a fatty aldehyde by a carboxylic acid reductase (CAR); or a fatty alcohol by a CAR and an alcohol dehydrogenase (ADH); or an α,ω-diol by a CAR, ADH, and an omega-hydroxylase/oxygenase; or an omega-hydroxy fatty acid by an omega-hydroxylase/oxygenase; or an α,ω-diacid by an omega-hydroxylase/oxygenase, an alcohol dehydrogenase/oxidase, and an aldehyde dehydrogenase/oxidase; or to an omega-amino fatty acid by an omega-hydroxylase/oxygenase; or an α,ω-diacid by an omega-hydroxylase/oxygenase, an alcohol dehydrogenase/oxidase, and an aminotransferase/transaminase. Alternatively, the acyl-CoA can be converted to a fatty ester by an ester synthase/acyltransferase, and the fatty ester can be converted to an omega-hydroxy fatty ester by an omega-hydroxylase/oxygenase; or to a mixed acid/ester (half acid ester) by an omega-hydroxylase/oxygenase, an alcohol dehydrogenase/oxidase, and an aldehyde dehydrogenase/oxidase; or to a mixed amino/ester by an omega-hydroxylase/oxygenase, an dehydrogenase/oxidase, and an alcohol aminotransferase/transaminase, etc.

As shown in FIG. 3 (and described elsewhere herein), the acyl-ACP-dependent fatty acid biosynthetic pathway can also lead to the generation of other undesired products (or byproducts), such as 3-oxo containing fatty acids and derivatives thereof, including, but not limited to, for example, 3-oxofatty esters (e.g., 3-oxo fatty acid methyl esters, 3-oxo fatty acid ethyl esters), 3-oxo fatty alcohols, and/or 3-oxo fatty alcohol acetates. As shown in FIG. 3, and described elsewhere herein, an acyl-ACP thioesterase can convert 3-keto-acyl-ACPs (produced in the fatty acid biosynthesis reductive cycle) to the corresponding 3-oxo fatty acids, which can be converted to the corresponding 3-oxo-acyl-CoAs by an acyl-CoA synthetase. The 3-oxo fatty acids can be converted by an (endogenous/native or heterologous) acyl-CoA synthetase to the corresponding 3-oxo-acyl-CoAs. As with the 3-OH fatty acids and 3-OH-acyl-CoAs, the 3-oxo fatty acids and 3-oxo-acyl-CoAs can be converted into various different 3-oxo group containing fatty acid derivative byproducts by one or more native or heterologous fatty acid derivative enzymes. For example, the 3-oxo fatty acids can be converted to 3-oxo fatty aldehydes by a CAR, which can then be converted to, for example, a 3-oxo fatty alcohol by an alcohol dehydrogenase, or to a 3-oxo fatty amine by a transaminase or aminotransferase. The 3-oxo fatty acids could also be converted to 3-hydroxy fatty acids and derivatives thereof by an enzyme capable of reducing the 3-oxo group to a 3-hydroxy group. The 3-oxo-acyl-CoAs can be converted to 3-oxo fatty acid esters by an ester synthase; or to 3-oxo fatty aldehydes by an acyl-CoA-reductase; or to 3-oxo fatty alcohols by an acyl-CoA-reductase and an alcohol dehydrogenase, or by a fatty-alcohol forming acyl-CoA reductase; or to 3-oxo fatty alcohol actetate esters by an acyl-CoA-reductase, an alcohol dehydrogenase, and an alcohol acetyl-CoA transferase, or by a fatty-alcohol forming acyl-CoA reductase and an alcohol acetyl-CoA transferase.

However, as shown in FIG. 3 (and described elsewhere herein), expression of a heterologous 3-oxoacyl-CoA reductase, β-ketoacyl-CoA-reductase, or 3-hydroxy acyl-CoA dehydrogenase (e.g., having EC 1.1.1.35, EC 1.1.1.36, or EC 1.1.1.157); and a heterologous R-3-hydroxy-acyl-CoA dehydratase or a heterologous R-specific enoyl-CoA hydratase, optionally in combination with a heterologous trans-2-enoyl-CoA reductase, can drive or increase the production of the non-3-oxo fatty acid derivatives (i.e., the fatty acids and derivatives thereof that do not contain a 3-oxo group, or the “target products”). The production of 3-oxo fatty acids and derivatives thereof (or 3-oxo group containing fatty acids and derivatives thereof), i.e., the “byproducts” is also reduced, eliminated, or substantially eliminated. For example, the 3-oxo-acyl-CoAs can be converted to 3-hydroxy-acyl-CoAs by one of a 3-oxoacyl-CoA reductase, β-ketoacyl-CoA-reductase, or 3-hydroxy acyl-CoA dehydrogenase (e.g., having EC 1.1.1.35, EC 1.1.1.36, or EC 1.1.1.157), and the 3-hydroxy-acyl-CoAs can be converted to 2-trans-enoyl-CoAs (alternatively termed trans-2-enoyl-CoAs) by either the R-3-hydroxy-acyl-CoA dehydratase (EC 4.2.1.134 or EC 4.2.1.55) or R-specific enoyl-CoA hydratase (EC 4.2.1.119 or EC 4.2.1.17). The 2-trans-enoyl-CoA is then reduced to the corresponding acyl-CoA by the trans-2-enoyl-CoA reductase (EC 1.3.1.44), and the acyl-CoA can then be converted to a fatty acid or a fatty acid derivative by the appropriate fatty acid derivative enzyme(s). For example, the acyl-CoA can be converted to: A) a fatty acid alkyl ester by an ester synthase (in the presence of an alcohol); B) a fatty alcohol by an acyl-CoA reductase and an alcohol dehydrogenase, or by a fatty-alcohol-forming acyl-CoA reductase; or C) a fatty alcohol acetate by the additional presence of an alcohol acetyl-CoA transferase (in the presence of acetyl-CoA) (in addition to the enzymes required for the production of a fatty alcohol). Other fatty acid derivatives can also be produced from the acyl-CoAs, as described elsewhere and as known in the art. For example, an acyl-CoA thioesterase can convert the acyl-CoA to the corresponding free fatty acid or an acyl-CoA reductase can convert the acyl-CoA to the corresponding fatty aldehyde. In some embodiments, an acyl-CoA thioesterase can convert the acyl-CoA to the corresponding free fatty acid, which can then be converted to a fatty aldehyde by a carboxylic acid reductase (CAR); or a fatty alcohol by a CAR and an alcohol dehydrogenase (ADH); or an α,ω-diol by a CAR, ADH, and an omega-hydroxylase/oxygenase; or an omega-hydroxy fatty acid by an omega-hydroxylase/oxygenase; or an α,ω-diacid by an omega-hydroxylase/oxygenase, an alcohol dehydrogenase/oxidase, and an aldehyde dehydrogenase/oxidase; or to an omega-amino fatty acid by an omega-hydroxylase/oxygenase; or an α,ω-diacid by an omega-hydroxylase/oxygenase, an alcohol dehydrogenase/oxidase, and an aminotransferase/transaminase. Alternatively, the acyl-CoA can be converted to a fatty ester by an ester synthase/acyltransferase, and the fatty ester can be converted to an omega-hydroxy fatty ester by an omega-hydroxylase/oxygenase; or to a mixed acid/ester (half acid ester) by an omega-hydroxylase/oxygenase, an alcohol dehydrogenase/oxidase, and an aldehyde dehydrogenase/oxidase; or to a mixed amino/ester by an omega-hydroxylase/oxygenase, an alcohol dehydrogenase/oxidase, and an aminotransferase/transaminase, etc.

In these modified biochemical pathways (or bypass pathways; see, FIG. 2 and FIG. 3), R-3-hydroxy acyl-CoA dehydratase and R-specific enoyl-CoA hydratase are interchangeable, as they can catalyze or carry out the same reversible enzymatic reaction, i.e., the reversible interconversion of a 3-hydroxy acyl-CoA and 2-trans-enoyl-CoA. Under certain physiological conditions such enzymes (i.e., a R-3-hydroxy acyl-CoA dehydratase) may favor the dehydration of a 3-hydroxy-acyl-CoA to a 2-trans-enoyl-CoA (or a trans-2-enoyl-CoA), or under certain physiological conditions, such an enzyme (i.e., a trans-2-enoyl-CoA hydratase) can favor the hydration of a 2-trans-enoyl-CoA to a 3-hydroxy acyl-CoA. In the pathways involving 3-oxo intermediates, the 3-oxoacyl-CoA reductase, β-ketoacyl-CoA-reductase, or 3-hydroxy acyl-CoA dehydrogenase are interchangeable, as they can catalyze or carry out the same reversible enzymatic reaction, i.e., the reversible interconversion of a 3-oxo-acyl-CoA and a 3-hydroxy acyl-CoA.

Since the bypass pathways depicted in both FIG. 2 and FIG. 3 include 3-hydroxy-acyl-CoA intermediates, that are converted to trans-2-enoyl-CoA intermediates by a R-3-hydroxy acyl-CoA dehydratase or a R-specific enoyl-CoA hydratase, both pathways can reduce or eliminate or substantially eliminate the production of 3-hydroxy fatty acid and 3-hydroxy fatty acid derivative byproducts. The addition of a 3-oxoacyl-CoA reductase, a β-ketoacyl-CoA-reductase, or a 3-hydroxy acyl-CoA dehydrogenase allows for the further reduction or elimination or substantial elimination of 3-oxo fatty acid and 3-oxo fatty acid derivative byproducts. Thus, the alternative or bypass pathway depicted in FIG. 2 can be used to reduce or eliminate 3-hydroxy byproducts, while the alternative or bypass pathway depicted in FIG. 3 can be used to reduce or eliminate both 3-oxo byproducts and 3-hydroxy byproducts.

Thus, the pathways and methods described herein effectively create a “bypass mechanism” to direct the biosynthetic pathway away from the production of 3-hydroxy fatty acids and derivatives thereof, and/or away from the production of 3-oxo fatty acids and derivatives thereof, and towards the conversion of the 3-hydroxy fatty acid and/or 3-oxo fatty acid byproducts (and precursors thereof, e.g., the 3-hydroxy-acyl-ACPs, 3-keto-acyl-ACPs, 3-hydroxy fatty acids, 3-oxo fatty acids, 3-oxo-acyl-CoAs, 3-hydroxy-acyl-CoAs, and/or trans-2-enoyl-CoAs) to the target products (that do not contain a 3-hydroxy or 3-oxo group), via acyl-CoA. Thus, the pathways, recombinant cells/microbes, and methods provided herein reduce or eliminate or substantially eliminate the byproducts, i.e., the 3-hydroxy group and/or 3-oxo group containing fatty acids and derivatives thereof, such as, e.g., 3-hydroxy fatty esters, 3-oxo fatty acids, 1,3 fatty diols, 3-oxo fatty alcohols, 3-oxo-fatty alcohol acetate esters, and/or fatty alcohol 1,3-diacetates, or other byproducts produced by the acyl-ACP dependent fatty acid biosynthesis pathway. In some exemplary embodiments, the “bypass mechanism” is employed to reduce, eliminate, or substantially eliminate byproducts of an acyl-ACP independent fatty acid biosynthesis pathway, wherein the pathway includes 3-hydroxy fatty acid and 3-hydroxy-acyl-CoA intermediates, and/or includes 3-oxo fatty acid, 3-oxo-acyl-CoA, and/or 3-hydroxy-acyl-CoA intermediates.

Elimination, substantial elimination, and/or reduction of 3-hydroxy and/or 3-oxo byproducts begins with the activation of 3-hydroxy fatty acids and/or 3-oxo fatty acids to the corresponding 3-hydroxy acyl-CoAs and 3-oxo acyl-CoAs, respectively, by acyl-CoA synthetase (EC 6.2.1.3). Such 3-hydroxy fatty acids and 3-oxo fatty acids are generated from 3-hydroxy-acyl-ACPs and 3-keto-acyl-ACPs, respectively, by the activity of an acyl-ACP thioesterase, which can be native to the cell, or can be heterologously expressed. In some embodiments, acyl-CoA synthetase activity that is native to the production host cell is sufficient to carry out the activation of 3-hydroxy fatty acids and/or 3-oxo fatty acids to the corresponding 3-hydroxy acyl-CoAs and 3-oxo-acyl-CoAs, respectively. Alternatively, the acyl-CoA synthetase can be heterologously expressed.

Additionally or alternatively, the pathways, recombinant cells/microbes, and/or methods provided herein can be used to reduce (or eliminate or substantially eliminate) byproducts created by any biochemical pathway that involves 3-hydroxy fatty acid, 3-oxo fatty acid, 3-oxo-acyl-CoA, and/or 3-hydroxy-acyl-CoA intermediates. For example, biosynthetic pathways for the production of a number of fatty acid derivative target products involve 3-hydroxy fatty acid, 3-oxo fatty acid, 3-oxo-acyl-CoA, and/or 3-hydroxy-acyl-CoA intermediates. Exemplary biosynthetic pathways that involve 3-hydroxy fatty acid, 3-oxo fatty acid, 3-oxo-acyl-CoA, and/or 3-hydroxy-acyl-CoA intermediates include, e.g., biosynthetic pathways for the production of fatty aldehydes (where the production of 3-hydroxy fatty aldehydes and.or 3-oxo fatty aldehydes is reduced, eliminated, or substantially eliminated), biosynthetic pathways for the production of fatty amines (where the production of 3-hydroxy fatty amines and/or 3-oxo fatty amines is reduced, eliminated, or substantially eliminated), biosynthetic pathways for the production of ω-hydroxy fatty esters (where the production of ω-hydroxy fatty esters with a 3-hydroxyl group or a 3-oxo group is reduced, eliminated, or substantially eliminated), biosynthetic pathways for the production of ω-carboxy fatty esters (where the production of ω-carboxy fatty esters with a 3-hydroxyl group or a 3-oxo group is reduced, eliminated, or substantially eliminated), biosynthetic pathways for the production of a/o-fatty diesters esters (where the production of α/ω-fatty diesters esters with a 3-hydroxyl group or 3-oxo group is reduced, eliminated, or substantially eliminated), etc.

The pathways, recombinant cells/microbes, and/or methods, provided herein, can also allow for the production of fatty acid derivatives of various chain lengths, e.g., even- or odd-chain C6 to C18, with various degrees of saturation or unsaturation (e.g., saturated and/or monounsaturated fatty acid derivatives), and with or without branching (e.g., straight-chain and/or branched-chain strain fatty acid derivatives), etc.

Thus, described herein is a recombinant cell, or microorganism, or microbe, comprising one or more of: A) an acyl-ACP thioesterase and an acyl-CoA synthetase; B) a heterologous R-3-hydroxy-acyl-CoA dehydratase or heterologous R-specific enoyl-CoA hydratase; and optionally, C) a heterologous trans-2-enoyl-CoA reductase. Also described herein is a recombinant cell, or microorganism, or microbe, comprising one or more of: A) an acyl-ACP thioesterase and an acyl-CoA synthetase; B) a heterologous a 3-oxoacyl-CoA reductase, a heterologous β-ketoacyl-CoA-reductase, or a heterologous 3-hydroxy acyl-CoA dehydrogenase; C) a heterologous R-3-hydroxy-acyl-CoA dehydratase or a heterologous R-specific enoyl-CoA hydratase; and optionally, D) a heterologous trans-2-enoyl-CoA reductase The acyl-ACP thioesterase and/or the acyl-CoA synthetase can be native (i.e., from the same species) to the recombinant cell, microbe, or microorganism, or can be heterologous (i.e., from a different species). A native enzyme can be endogenously expressed (i.e., the encoding gene or nucleic acid sequence is from within the cell) or exogenously expressed (i.e., the encoding gene or nucleic acid sequence is added to the cell from outside the cell). Additionally or alternatively, a native enzyme can be expressed or it can be overexpressed.

The trans-2-enoyl-CoA reductase can be from Treponema denticola or from Euglena gracilis. Alternatively, the trans-2-enoyl-CoA reductase can be any one described in Table 2 (and/or in any one of SEQ ID NOs: 14-25), or can be any other enzyme that functions as a trans-2-enoyl-CoA reductase, i.e., any polypeptide or enzyme with activity to convert trans-2-enoyl-CoA to the corresponding acyl-CoA, and/or any polypeptide or enzyme that can be described by EC 1.3.1.44. Additionally or alternatively, the trans-2-enoyl-CoA reductase can be TER or FabV from Treponema denticola, Euglena gracilis, Enterovibrio coralii, Lactobacillus oligofermentans, Paucilactobacillus oligofermentans, Vibrio spp., Treponema leithinolyticum, Treponema pedis, Treponema vincentii, Treponema sp., Treponema brennaborense, or Treponema parvum. One skilled in the art can easily determine if an enzyme or polypeptide has this function.

In a particular embodiment, the R-3-hydroxy-acyl-CoA dehydratase or R-specific enoyl-CoA hydratase can be PhaJ1, PhaJ3, PhaJ4, MaoC9, or MaoC (or an MaoC family dehydratase). Alternatively, the R-3-hydroxy-acyl-CoA dehydratase or R-specific enoyl-CoA hydratase may be any one described in Table 1 (and/or in any one of SEQ ID NOs: 1-13), or can be any other enzyme or polypeptide that functions as a R-3-hydroxy-acyl-CoA dehydratase or a R-specific enoyl-CoA hydratase, i.e., one that has activity to convert a 3-hydroxy-acyl-CoA to a trans-2-enoyl-CoA, and/or any polypeptide or enzyme that can be described by EC 4.2.1.119 or EC 4.2.1.17. One skilled in the art can easily determine if an enzyme or polypeptide has this function.

In some embodiments, the 3-oxoacyl-CoA reductase, β-ketoacyl-CoA-reductase, or 3-hydroxy acyl-CoA dehydrogenase may be from Ralstonia eutropha (Cupriavidus necator), Cupriavidus taiwanensis, Bacillus, Burkholderia, Pseudomonas, or Megasphaera. Alternatively, the 3-oxoacyl-CoA reductase, β-ketoacyl-CoA-reductase, or 3-hydroxy acyl-CoA dehydrogenase can be any one described in Table 2A (and/or in any one of SEQ ID NOs: 38-45), or can be any other enzyme that functions as a 3-oxoacyl-CoA reductase, β-ketoacyl-CoA-reductase, or 3-hydroxy acyl-CoA dehydrogenase, i.e., any polypeptide or enzyme with activity to convert a 3-oxo-acyl-CoA to the corresponding 3-hydroxy-acyl-CoA, and/or any polypeptide or enzyme that can be described by EC 1.1.1.35, EC 1.1.1.36, or EC 1.1.1.157. For example, the 3-oxoacyl-CoA reductase, β-ketoacyl-CoA-reductase, or 3-hydroxy acyl-CoA dehydrogenase may be from, but are not limited to, for example, Cupriavidus necator, Ralstonia eutropha, Cupriavidus necator H16, Cupriavidus taiwanensis, Bacillus sp. SG-1, Burkholderia multivorans, Pseudomonas putida, or Megasphaera elsdenii.

One skilled in the art can easily determine if an enzyme or polypeptide has this function.

In some embodiments, the recombinant cell or microbe further comprises one or more additional enzymes for the production of fatty acids and derivatives thereof (i.e., one or more fatty acid derivative enzymes and/or one or more fatty acid biosynthetic/biosynthesis enzymes), including, but not limited to, an ester synthase, a β-keto-acyl-ACP synthase (I, II, and/or III), an acyl-CoA thioesterase, an acyl-ACP thioesterase, an acyl-CoA synthetase, an alcohol dehydrogenase, an alcohol-O-acetyl-transferase, an acyl-CoA reductase, a fatty-alcohol-forming acyl-CoA reductase, a carboxylic acid reductase, a desaturase, an omega-hydroxylase, a transaminase (or aminotransferase), an amine dehydrogenase, a CoA-ligase/transferase, an alcohol-O-acetyl transferase, an aldehyde decarbonylase, an aldehyde oxidative deformylase, a decarboxylase, one or more subunits (e.g., AccA, AccB, AccC, and/or AccD) of an acetyl-CoA carboxylase (AccABCD), an OleA, an OleBCD, an OleABCD, an OleACD, an aldehyde dehydrogenase, and/or FadR. One or more (or all) of the additional enzymes can be native or heterologous, and can be endogenous/endogenously expressed or exogenous/exogenously expressed.

The recombinant cell or microbe may be a bacterium, cyanobacterium, yeast, or algae. When the recombinant cell or microbe is a bacterium, it may be a y-proteobacterium, such as Escherichia coli, Salmonella spp., Vibrio natriegens, Pseudomonas aeruginosa, Pseudomonas putida, Pseudomonas fluorescens, Xanthomonas axonopodis, Pseudomonas syringae, Pseudomonas citronellolis, Pseudomonas mendocina, Pseudomonas plecoglossicida, Pseudomonas mosselii, Pseudomonas fulva, Xyella fastidiosa, Marinobacter aquaeolei, Yersinia pestis, Bacillus spp., Lactobacillus spp., Zymomonas spp., Streptomyces spp., or Vibrio cholerae. In particular, the y-proteobacterium may be Escherichia coli.

Additionally or alternatively, the recombinant cell or microbe may be a cyanobacterium, such as, for example, Synechococcus elongatus PCC7942 or Synechocystis sp. PCC6803.

Additionally or alternatively, the recombinant cell or microbe may be a yeast, such as, for example, Saccharomyces cerevisiae, Scheffersomyces stipitis, Schizosaccharomyces pombe, Kluyveromyces marxianus, K. lactis, Pichia pastoris, Hansenula polymorpha, or Yarrowia lipolytica.

Additionally or alternatively, the recombinant cell or microbe may be an algae, such as, for example, Botryococcus braunii, Nannochloropsis gaditina, Chlamydomonas reinhardtii, Chlorella vulgaris., Spirulina platensis, Ostreococcus tauri, Phaeodactylum tricornutum, Symbiodinium sp., algal phytoplanktons, cyanobacteria mats, Saccharina japonica, Chlorococum spp., or Spirogyra spp.

Additionally or alternatively, the recombinant cell or microbe can comprise a heterologous acyl-ACP thioesterase from Umbellularia californica (e.g., FatB1) or a heterologous acyl-ACP thioesterase from Cuphea hookeriana (e.g., FatB2). Additionally or alternatively, the recombinant cell or microbe can comprise the heterologous R-specific enoyl-CoA hydratase PhaJ1 from Pseudomonas putida, PhaJ4 from P. putida, PhaJ3 from Pseudomonas aeruginosa, or PhaJ4 from P. aeruginosa. Additionally or alternatively, the recombinant cell or microbe may be E. coli, comprising a heterologous acyl-ACP thioesterase from Umbellularia californica (e.g., FatB1) or a heterologous acyl-ACP thioesterase from Cuphea hookeriana (e.g., FatB2), a native acyl-CoA synthetase that is overexpressed (FadD) or a heterologous acyl-CoA synthetase from Pseudomonas putida (e.g., FadD3), and a heterologous R-specific enoyl-CoA hydratase, such as one of PhaJ1 from Pseudomonas putida, PhaJ4 from P. putida, PhaJ3 from Pseudomonas aeruginosa, or PhaJ4 from P. aeruginosa. In some embodiments, these recombinant cells or microbes do not contain a heterologous trans-2-enoyl-CoA reductase. In some embodiments, the recombinant cell or microbe additionally comprises a heterologous 3-oxoacyl-CoA reductase, β-ketoacyl-CoA-reductase, or 3-hydroxy acyl-CoA dehydrogenase, such as one from Ralstonia eutropha (Cupriavidus necator), Cupriavidus taiwanensis, Bacillus, Burkholderia, Pseudomonas, or Megasphaera. Additionally, the recombinant cell or microbe may further comprise an ester synthase, a β-keto-ACP-synthase I (FabB), and FadR, any of which may be native or heterologous. FadE expression can also be optionally attenuated or deleted in the recombinant cell or microbe, as compared to a corresponding control or reference or wildtype cell or microbe.

Additionally or alternatively, the recombinant cell or microbe may be E. coli comprising the heterologous acyl-ACP thioesterase FatB1 from Umbellularia californica, the heterologous acyl-CoA synthetase FadD3 that is overexpressed, the heterologous R-specific enoyl-CoA hydratase PhaJ4 from P. putida, and the heterologous trans-2-enoyl-CoA reductase TER from T. denticola. In some embodiments, the recombinant cell or microbe may further comprise a heterologous 3-oxoacyl-CoA reductase, β-ketoacyl-CoA-reductase, or 3-hydroxy acyl-CoA dehydrogenase, such as one from Ralstonia eutropha (Cupriavidus necator), Cupriavidus taiwanensis, Bacillus, Burkholderia, Pseudomonas, or Megasphaera. Additionally, the recombinant cell or microbe may further comprise a heterologous ester synthase, and FadR, and FadE expression can optionally be attenuated or deleted in the recombinant cell microbe, as compared to a corresponding control or reference or wildtype cell or microbe.

Additionally or alternatively, the recombinant cell or microbe may be E. coli comprising a heterologous acyl-ACP thioesterase (e.g., FatB2) from Cuphea hookeriana, aheterologous acyl-CoA synthetase (e.g., FadD3) from P. putida, a heterologous R-specific enoyl-CoA hydratase (e.g., PhaJ4) from P. putida, and a heterologous trans-2-enoyl-CoA reductase (e.g., TER) from T. denticola. In some embodiments, the recombinant cell or microbe may further comprise a heterologous 3-oxoacyl-CoA reductase, β-ketoacyl-CoA-reductase, or 3-hydroxy acyl-CoA dehydrogenase, such as one from Ralstonia eutropha (Cupriavidus necator), Cupriavidus taiwanensis, Bacillus, Burkholderia, Pseudomonas, or Megasphaera. Additionally, the recombinant cell or microbe may further comprise a heterologous ester synthase, and an overexpressed FadR, and FadE expression can optionally be attenuated or deleted in the recombinant cell or microbe as compared to a corresponding control or reference or wildtype cell or microbe.

Additionally or alternatively, the recombinant cell or microbe may comprise acyl-CoA synthetase (FadD) from E. coli and/or FadD3 from P. putida. Additionally or alternatively, the recombinant cell or microbe may be E. coli comprising a heterologous acyl-ACP thioesterase from C. hookeriana or U. californicia, a deregulated (such as an attenuated or deleted), native acyl-CoA synthetase (FadD) and/or an overexpressed heterologous acyl-CoA synthetase (e.g., FadD3 from P. putida), a heterologous R-specific enoyl-CoA hydratase (e.g., PhaJ from P. putida or P. aeruginosa), and a heterologous trans-2-enoyl-CoA reductase (e.g., TER from Treponema denticola). In some embodiments, the recombinant cell or microbe may further comprise a heterologous 3-oxoacyl-CoA reductase, β-ketoacyl-CoA-reductase, or 3-hydroxy acyl-CoA dehydrogenase, such as one from Ralstonia eutropha (Cupriavidus necator), Cupriavidus taiwanensis, Bacillus, Burkholderia, Pseudomonas, or Megasphaera. Additionally, the recombinant cell or microbe may further comprise a heterologous carboxylic acid reductase and a heterologous alcohol dehydrogenase, and FadE expression can optionally be attenuated or deleted in the recombinant cell or microbe, as compared to a corresponding control or reference or wildtype cell or microbe. Alternatively, the recombinant cell or microbe may further comprise a heterologous acyl-CoA reductase, a heterologous carboxylic acid reductase, a heterologous alcohol dehydrogenase, and a heterologous alcohol-O-acetyl transferase.

The recombinant cells or microbes described above can produce one or more fatty acid derivatives and/or a composition comprising one or more fatty acid derivatives, where the fatty acid derivatives may be, but are not limited to, for example, free fatty acids, fatty esters, fatty alcohols, fatty alcohol acetates, fatty alcohol acetate esters, fatty aldehydes, fatty amines, fatty amides, fatty diols, fatty triols, fatty tetrols, ω-hydroxy fatty acids, ω-hydroxy fatty esters, ω-carboxy fatty acids, ω-carboxy fatty esters, α,ω-fatty diacids, α,ω-fatty diols, and α,ω-fatty diesters. Additionally, the recombinant cells or microbes can produce a reduced amount (relatively less) of byproducts, such as 3-hydroxy fatty acids and derivatives thereof (including, e.g., 3-hydroxy fatty acid esters, 1,3-diols, and/or fatty alcohol 1,3-diacetates (1,3-fatty diol acetates)), as compared to cells or microbes that do not possess, or express, or comprise one of a heterologous R-3-hydroxy-acyl-CoA dehydratase or a heterologous R-specific enoyl-CoA hydratase; and also may not possess, express, or comprise a heterologous trans-2-enoyl-CoA reductase. In some embodiments, the recombinant cells or microbes can produce a reduced amount (relatively less) of 3-hydroxy fatty acids and derivatives thereof and/or a reduced amount of trans-2-fatty acids and derivatives thereof, as compared to cells or microbes that do not possess, or express, or comprise a heterologous R-3-hydroxy-acyl-CoA dehydratase or a heterologous R-specific enoyl-CoA hydratase; and also do not possess, express, or comprise a heterologous trans-2-enoyl-CoA reductase. In some embodiments, the recombinant cells or microbes do not produce any byproducts, such as 3-hydroxy fatty acids and derivatives thereof. In some embodiments, the 3-hydroxy fatty acids and derivatives thereof may be, but are not limited to, for example, 3-hydroxy fatty acids; 3-hydroxy fatty acid esters; 1,3-diols; fatty alcohol 1,3-diacetates (1,3-fatty diol acetates); 3-hydroxy fatty aldehydes; 3-hydroxy fatty amines; 3-hydroxy fatty amides; fatty diols with a 3-hydroxy group; fatty triols with a 3-hydroxy group; fatty tetrols with a 3-hydroxy group; ω-hydroxy fatty acids with a 3-hydroxy group, ω-carboxy fatty acids with a 3-hydroxy group, ω-hydroxy fatty esters with a 3-hydroxy group, ω-carboxy fatty esters with a 3-hydroxy group, α,ω-fatty diacids with a 3-hydroxy group, α,ω-fatty diesters with a 3-hydroxy group, and/or α,ω-fatty diols with a 3-hydroxy group. In some embodiments, the trans-2-fatty acids or derivatives thereof may be, but are not limited to, for example, trans-2-fatty acids; trans-2-fatty acid esters; trans-2-fatty alcohols; trans-2-fatty alcohol acetates; trans-2-fatty aldehydes; trans-2-fatty amines; trans-2-fatty amides; trans-2-fatty 1,3-diols; trans-2-fatty diols; trans-2-fatty triols; trans-2-fatty tetrols; trans-2-ω-hydroxy fatty acids, trans-2-ω-carboxy fatty acids, trans-2-ω-hydroxy fatty esters; trans-2-ω-carboxy fatty esters; trans-2-α,ω-fatty diacids; trans-2-α,ω-fatty diesters; and/or trans-2-α,ω-fatty diols. Additionally, the recombinant cells or microbes can produce a reduced amount (relatively less) of byproducts, such as 3-oxo fatty acids and derivatives thereof (including, e.g., 3-oxo fatty acid esters, 3-oxo fatty alcohols, and/or 3-oxo fatty alcohol acetates), as compared to cells or microbes that do not possess, or express, or comprise a heterologous 3-oxoacyl-CoA reductase, a heterologous β-ketoacyl-CoA-reductase, or a heterologous 3-hydroxy acyl-CoA dehydrogenase; and a heterologous R-3-hydroxy-acyl-CoA dehydratase or a heterologous R-specific enoyl-CoA hydratase; and also may not possess, express, or comprise a heterologous trans-2-enoyl-CoA reductase. In some embodiments, the recombinant cells or microbes can produce a reduced amount (relatively less) of as 3-oxo fatty acids and derivatives thereof and/or a reduced amount of trans-2-fatty acids and derivatives thereof, as compared to cells or microbes that do not possess, or express, or comprise a comprise a heterologous 3-oxoacyl-CoA reductase, a heterologous β-ketoacyl-CoA-reductase, or a heterologous 3-hydroxy acyl-CoA dehydrogenase; and a heterologous R-3-hydroxy-acyl-CoA dehydratase or a heterologous R-specific enoyl-CoA hydratase; and also do not possess, express, or comprise a heterologous trans-2-enoyl-CoA reductase. In some embodiments, the recombinant cells or microbes do not produce any byproducts, such as 3-oxo fatty acids and derivatives thereof. In some embodiments, the 3-oxoacyl-CoA reductases may be, but are not limited to, for example, β-ketoacyl-CoA-reductases, and/or the 3-hydroxy acyl-CoA dehydrogenases may be, but are not limited to, for example, PhaB, HbD, PaaH1, FabG, PhhB, and/or MELS_1448. In some embodiments, the 3-oxo fatty acids or derivatives thereof may be, but are not limited to, for example, 3-oxo fatty acids; 3-oxo fatty acid esters; 3-oxo fatty alcohols; 3-oxo fatty alcohol acetate esters; 3-oxo fatty aldehydes; 3-oxo fatty amines; 3-oxo fatty amides; 3-oxo fatty diols; 3-oxo fatty triols; 3-oxo fatty tetrols; ω-hydroxy fatty acids with a 3-oxo group; ω-carboxy fatty acids with a 3-oxo group; ω-hydroxy fatty esters with a 3-oxo group; ω-carboxy fatty esters with a 3-oxo group; α,ω-fatty diacids with a 3-oxo group; α,ω-fatty diesters with a 3-oxo group; and/or α,ω-fatty diols with a 3-oxo group.

Additionally or alternatively, the recombinant cell or microbe may produce a plurality of fatty acids or derivatives thereof, or a composition comprising the same, that is/are free or substantially free, as defined herein, of unwanted byproducts, such as 3-hydroxy, trans-2-fatty acids, and/or 3-oxo fatty acids and derivatives thereof.

In some exemplary embodiments, the host cell (e.g., a recombinant microbe; or a recombinant bacterium, proteobacterium, cyanobacterium, yeast, or algae) may further comprise genetic manipulations and alterations to enhance or otherwise fine tune the production of the target fatty acids or derivatives thereof. The optional genetic manipulations can be used interchangeably from one host cell to another, depending on what other heterologous enzymes and what native enzymatic pathways are present in the host cell. Some optional genetic manipulations include one or more of the following modifications described below.

The gene encoding acyl-CoA dehydrogenase (e.g., FadE) can optionally be attenuated or deleted in the recombinant cells, microbes, or microorganisms provided herein. FadE (Acyl-CoA dehydrogenase) catalyzes the first step in fatty acid utilization/degradation (β-oxidation cycle), which is the oxidation of acyl-CoA to 2-enoyl-CoA (see e.g., Campbell, J. W. and Cronan, J. E. Jr (2002) J. Bacteriol. 184 (13): 3759-3764; and Lennen, R. M. and Pfleger, B. F (2012) Trends Biotechnol. 30 (12): 659-667). Since FadE initiates the β-oxidation cycle, when E. coli lacks FadE, it cannot grow on fatty acids as a carbon source (see e.g., Campbell, J. W. and Cronan supra). The same effect can be achieved by attenuating or deleting other enzymes from the B-oxidation cycle, e.g., FadA, which is a 3-ketoacyl-CoA thiolase, or FadB, which is a dual 3-hydroxyacyl-CoA-dehydrogenase/dehydratase.

However, when a microbe such as E. coli is grown on a carbon source other than fatty acids, e.g., when it is grown on sugar, acetate, etc., FadE attenuation is optional, because under such conditions, FadE expression is repressed by FadR. Therefore, when cells are grown on a simple carbon source, such as, e.g., glucose, the FadE gene product is already attenuated. Accordingly, when grown on a carbon source other than fatty acids, a FadE mutation/deletion or attenuation is optional.

In some embodiments, the fatty acid biosynthetic pathway in the production host uses the precursors acetyl-CoA and malonyl-CoA. E. coli or other host organisms engineered to overproduce these components can serve as the starting point for subsequent genetic engineering steps to provide the specific output product (such as, fatty acids, fatty esters, hydrocarbons, fatty alcohols, etc.). Several different modifications can be made, either in combination or individually, to the host cell or strain, to obtain increased acetyl-CoA, malonyl-CoA, fatty acid, and/or fatty acid derivative production. See, for example, U.S. Patent Application Publication 2010/0199548, which is incorporated herein by reference in its entirety. For example, to increase malonyl-CoA production, one or more of the acetyl-CoA carboxylase subunits, including AccA, AccB, AccC, and/or AccD, can be expressed or overexpressed in the recombinant cell or microbe.

Other exemplary modifications of a host cell include, e.g., overexpression of non-native and/or native and/or variants of genes involved in the synthesis of acyl-ACP. In general, increasing acyl-ACP synthesis increases the amount of acyl-ACP, which is the substrate of thioesterases, ester synthases, and acyl-ACP reductases. Exemplary enzymes that increase acyl-ACP production include, e.g., enzymes that make up the “fatty acid synthase” (FAS). As is known in the art (see e.g., U.S. 2010/0199548) FAS enzymes are a group of enzymes that catalyze the initiation and elongation of acyl chains. The acyl carrier protein (ACP) along with the enzymes in the FAS pathway control the length, degree of saturation, and branching of the fatty acids produced. FAS pathway enzymes include, for example, AccABCD, FabD, FabH, FabG, FabA, FabB, FabZ, FabF, FabI, FabK, FabL, FabM, FabQ, FabV, FabX, FabR, and FadR (see, e.g., Table A below for a description of these and other enzymes), and homologs thereof and corresponding enzymes with the same activities that are derived from other organisms or species. Depending upon the desired product, one or more of these genes can be attenuated, deleted, downregulated, expressed, upregulated, or over-expressed, or otherwise modified or deregulated. The functions or exemplary uses for FAS genes (e.g., accA, accB, accC, accD, fabA, fabB, fabD, fabF, fabG, fabH, fabl, fabR, fabV, fabZ, fabK, fabL, fabM, fabX) are provided in Table A below. Table A also provides the functions or exemplary uses genes encoding other enzymes, including, for example, certain fatty acid derivative genes (e.g., acyl-CoA synthetases, thioesterases, ester synthases, alcohol dehydrogenases, acyl-CoA reductases, etc.). Any one or more of the genes listed in Table A can be expressed or overexpressed in the recombinant cells or microbes provided herein, including heterologously expressed or overexpressed. Additionally or alternatively, the expression or activity of any one or more of the genes listed in Table A can be altered, deregulated, or modified, for example, by attenuation, downregulation, or deletion of one or more genes and their encoded products.

In some embodiments, the recombinant cells, microbes, or microorganisms provided herein contain pathways that use a renewable feedstock, such as glucose, to produce fatty acids and derivatives thereof. Glucose is converted to an acyl-ACP by the native organism. Polynucleotides that code for polypeptides with fatty acid degradation enzyme activity can be optionally attenuated depending on the desired product. Non-limiting examples of such polypeptides are acyl-CoA synthetase (FadD) and acyl-CoA dehydrogenase (FadE). The Table below (Table A) provides a comprehensive list of enzymatic activity (infra) within the metabolic pathway, including various fatty acid degradation enzymes that can be optionally attenuated according to methods known in the art (see, e.g., U.S. Pat. No. 8,283,143).

For example, FadR (see Table A, infra) is a key regulatory factor involved in fatty acid degradation and fatty acid biosynthetic pathways (Cronan et al., Mol. Microbiol., 29 (4): 937-943 (1998)). The E. coli enzyme FadD (see Table 1, infra) and the fatty acid transport protein FadL are components of a fatty acid uptake system. FadL mediates transport of fatty acids into the bacterial cell, and FadD mediates formation of acyl-CoA esters. When no other carbon source is available, exogenous fatty acids are taken up by bacteria and converted to acyl-CoA esters, which can bind to the transcription factor FadR and depress the expression of the fad genes that encode proteins responsible for fatty acid transport (FadL), activation (FadD), and β-oxidation (FadA, FadB, and FadE,). When alternative sources of carbon are available, bacteria synthesize fatty acids as acyl-ACPs, which are used for phospholipid synthesis, but are not substrates for β-oxidation. Thus, acyl-CoA and acyl-ACP are both independent sources of fatty acids that can result in different end-products (Caviglia et al., J. Biol. Chem., 279 (12): 1163-1169 (2004)).

TABLE A
Enzymatic Activities

| | | | | | |
|---|---|---|---|---|---|
| Gene | Source | | | | |
| Designation | Organism | Enzyme Name | Accession # | EC Number | Exemplary Use |

Fatty Acid Production Increase

| | | | | | |
|---|---|---|---|---|---|
| accA | E. coli, | Acetyl-CoA | AAC73296, | 6.4.1.2 | increase Malonyl-CoA |
| | Lactococci | carboxylase, subunit A | NP_414727 | | production |
| | | (carboxyltransferase | | | |
| | | alpha) | | | |
| accB | E. coli, | Acetyl-CoA | NP_417721 | 6.4.1.2 | increase Malonyl-CoA |
| | Lactococci | carboxylase, subunit B | | | production |
| | | (BCCP: biotin carboxyl | | | |
| | | carrier protein) | | | |
| accC | E. coli, | Acetyl-CoA | NP_417722 | 6.4.1.2, | increase Malonyl-CoA |
| | Lactococci | carboxylase, subunit C | | 6.3.4.14 | production |
| | | (biotin carboxylase) | | | |
| accD | E. coli, | Acetyl-CoA | NP_416819 | 6.4.1.2 | increase Malonyl-CoA |
| | Lactococci | carboxylase, subunit D | | | production |
| | | (carboxyltransferase | | | |
| | | beta) | | | |
| fadD | E. coli W3110 | acyl-CoA synthetase | AP_002424 | 6.2.1.3 | increase Fatty acid |
| | | | | | production (when acyl- |
| | | | | | CoA intermediates are |
| | | | | | involved) |
| fabA | E. coli K12 | β-hydroxydecanoyl | NP_415474 | 4.2.1.60 | increase fatty acyl- |
| | | thioester | | | |
| | | dehydratase/isomerase | | | ACP/CoA production |
| fabB | E. coli | 3-oxoacyl-[acyl-carrier- | BAA16180 | 2.3.1.41 | increase fatty acyl- |
| | | protein] synthase I | | | ACP/CoA production |
| fabD | E. coli K12 | [acyl-carrier-protein] | AAC74176 | 2.3.1.39 | increase fatty acyl- |
| | | S-malonyltransferase | | | ACP/CoA production |
| fabF | E. coli K12 | 3-oxoacyl-[acyl- | AAC74179 | 2.3.1.179 | increase fatty acyl- |
| | | carrier-protein] | | | ACP/CoA production |
| | | synthase II | | | |
| fabG | E. coli K12 | 3-oxoacyl-[acyl- | AAC74177 | 1.1.1.100 | increase fatty acyl- |
| | | carrier protein] | | | ACP/CoA production |
| | | reductase | | | |
| fabH | E. coli K12 | 3-oxoacyl-[acyl- | AAC74175 | 2.3.1.180 | increase fatty acyl- |
| | | carrier-protein] | | | ACP/CoA production |
| | | synthase III | | | |
| fabI | E. coli K12 | enoyl-[acyl-carrier- | NP_415804 | 1.3.1.9 | increase fatty acyl- |
| | | protein] reductase | | | ACP/CoA production |
| fabR | E. coli K12 | Transcriptional | NP_418398 | none | modulate unsaturated |
| | | Repressor | | | fatty acid production |
| fabV | Vibrio cholerae | enoyl-[acyl-carrier- | YP_001217283 | 1.3.1.9 | increase fatty acyl- |
| | | protein] reductase | | | ACP/CoA production |
| fabZ | E. coli K12 | (3R)- | NP_414722 | 4.2.1.— | increase fatty acyl- |
| | | hydroxymyristol acyl | | | ACP/CoA production |
| | | carrier protein | | | |
| | | dehydratase | | | |
| fadE | E. coli K13 | acyl-CoA | AAC73325 | 1.3.99.3, | reduce fatty acid |
| | | dehydrogenase | | 1.3.99.— | degradation (if |
| | | | | | attenuated) |
| fadD | E. coli K12 | acyl-CoA synthetase | NP_416319 | 6.2.1.3 | reduce fatty acid |
| | | | | | degradation (if |
| | | | | | attenuated) |
| fadA | E. coli K12 | 3-ketoacyl-CoA | YP_02627 | 2.3.1.16 | reduce fatty acid |
| | | thiolase | | | degradation (if |
| | | | | | attenuated) |
| fadB | E. coli K12 | enoyl-CoA hydratase | NP_418288 | 4.2.1.17, | reduce fatty acid |
| | | 3-OH acyl-CoA | | 5.1.2.3, | degradation (if |
| | | epimerase/ | | 1.1.1.35 | attenuated) |
| | | dehydrogenase | | | |
| fadR | E. coli | transcriptional | NP_415705 | none | enhances fatty acid |
| | | regulatory protein | | | biosynthesis when |
| | | | | | upregulated, and enhances |
| | | | | | fatty acid degradation when |
| | | | | | attenuated |

Chain Length Control

| | | | | | |
|---|---|---|---|---|---|
| tesA (with or | E. coli | thioesterase - leader | P0ADA1 | 3.1.2.—, | C14-C18 Chain Length |
| without leader | | sequence is amino | | 3.1.1.5 | |
| sequence) | | acids 1-26 | | | |
| tesA (without | E. coli | thioesterase | AAC73596, | 3.1.2.—, | C14-C18 Chain Length |
| leader | | | NP_415027 | 3.1.1.5 | |
| sequence) | | | | | |
| fatB1 | Umbellularia | thioesterase | Q41635 | 3.1.2.14 | C12 Chain Length |
| | californica | | | | |
| fatB2 | Cuphea | thioesterase | AAC49269 | 3.1.2.14 | C8-C10 Chain Length |
| | hookeriana | | | | |
| fatB3 | Cuphea | thioesterase | AAC72881 | 3.1.2.14 | C14-C16 Chain Length |
| | hookeriana | | | | |
| fatB | Cuphea | thioesterase | CAB60830 | 3.1.2.14 | C8-C10 Chain Length |
| | lanceolata | | | | |
| fatB | Cinnamomumcan | thioesterase | Q39473 | 3.1.2.14 | C14 Chain Length |
| | phora | | | | |
| fatB | Arabidopsis | thioesterase | CAA85388 | 3.1.2.14 | C16 Chain Length |
| | thaliana | | | | |
| fatB1 | Umbellularia | thioesterase | Q41635 | 3.1.2.14 | C12 Chain Length |
| | californica | | | | |
| fatA1 | Helianthus | thioesterase | AAL79361 | 3.1.2.14 | C16:1, C18:1 Chain |
| | annuus | | | | Length |
| fatA | Arabidopsis | thioesterase | NP_189147, | 3.1.2.14 | C16:1, C18:1 Chain |
| | thaliana | | NP_193041 | | Length |
| fatA | Brassica juncea | thioesterase | CAC39106 | 3.1.2.14 | C16:1, C18:1 Chain |
| | | | | | Length |
| fatA | Cuphea | thioesterase | AAC72883 | 3.1.2.14 | C16:1, C18:1 Chain |
| | hookeriana | | | | Length |
| tesB | E. coli | thioesterase | NP_414986 | 3.1.2.14 | deletion or attenuation |
| | | | | | increases FAS derivatives |
| | | | | | when acyl-CoA |
| | | | | | intermediates are involved |
| fadM | E. coli | thioesterase | NP_414977 | 3.1.2.14 | deletion or attenuation |
| | | | | | increases FAS derivatives |
| | | | | | when acyl-CoA |
| | | | | | intermediates are involved |
| yciA | E. coli | thioesterase | NP_415769 | 3.1.2.14 | deletion or attenuation |
| | | | | | increases FAS derivatives |
| | | | | | when acyl-CoA |
| | | | | | intermediates are involved |
| ybgC | E. coli | thioesterase | NP_415264 | 3.1.2.14 | deletion or attenuation |
| | | | | | increases FAS derivatives |
| | | | | | when acyl-CoA |
| | | | | | intermediates are involved |

Saturation Level Control

| | | | | | |
|---|---|---|---|---|---|
| fabA | E. coli K12 | β-hydroxydecanoyl | NP_415474 | 4.2.1.60 | produce unsaturated fatty |
| | | thioester | | | acids |
| | | dehydratase/isomerase | | | |
| fabB | E. coli | 3-oxoacyl-[acyl- | BAA16180 | 2.3.1.41 | modulate unsaturated |
| | | carrier-protein] | | | fatty acid production |
| | | synthase I | | | |
| des | Bacillus subtilis | D5 fatty acyl | O34653 | 1.14.19.— | modulate unsaturated |
| | | desaturase | | | fatty acid production |
| fabX | H. pylori | — | Uniprot | undefined | increase unsaturation |
| | | | O25465 | | |

Ester Production

| | | | | | |
|---|---|---|---|---|---|
| AT3G51970 | Arabidopsis | long-chain-alcohol | NP_190765 | 2.3.1.26 | ester production |
| | thaliana | O-fatty- | | | |
| | | acyltransferase | | | |
| ELO1 | Pichia angusta | Fatty acid elongase | BAD98251 | 2.3.1.— | produce very long chain |
| | | | | | length fatty acids |
| plsC | Saccharomyces | acyltransferase | AAA16514 | 2.3.1.51 | ester production |
| | cerevisiae | | | | |
| DAGAT/DGAT | Arabidopsis | diacylglycerol | AAF19262 | 2.3.1.20 | ester production |
| | thaliana | acyltransferase | | | |
| hWS | Homo sapiens | acyl-CoA wax | AAX48018 | 2.3.1.20 | ester production |
| | | alcohol | | | |
| | | acyltransferase | | | |
| aft1 | Acinetobacter sp. | bifunctional wax ester | AAO17391 | 2.3.1.20 | ester production |
| | ADP1 | synthase/acyl- | | | |
| | | CoA: diacylglycerol | | | |
| | | acyltransferase | | | |
| ES9 | Marinobacter | wax ester synthase | ABO21021 | 2.3.1.20 | ester production |
| | hydrocarbonoclasticus | | | | |
| mWS | Simmondsia | wax ester synthase | AAD38041 | 2.3.1.— | ester production |
| | chinensis | | | | |

Fatty Alcohol Output

| | | | | | |
|---|---|---|---|---|---|
| | | thioesterases (see | | | increase fatty acid/fatty |
| | | above) | | | alcohol production |
| BmFAR | Bombyxmori | FAR (fatty alcohol | BAC79425 | 1.1.1.— | convert acyl-CoA to fatty |
| | | forming acyl-CoA | | | alcohol |
| | | reductase) | | | |
| acr1 | Acinetobacter sp. | acyl-CoA reductase | YP_047869 | 1.2.1.42 | reduce fatty acyl-CoA to |
| | ADP1 | | | | fatty aldehydes |
| yqhD | E. coli W3110 | alcohol | AP_003562 | 1.1.—.— | reduce fatty aldehydes to |
| | | dehydrogenase | | | fatty alcohols; increase |
| | | | | | fatty alcohol production |
| alrA | Acinetobacter sp. | alcohol | CAG70252 | 1.1.—.— | reduce fatty aldehydes to |
| | ADP1 | dehydrogenase | | | fatty alcohols |
| BmFAR | Bombyxmori | FAR (fatty alcohol | BAC79425 | 1.1.1.— | reduce fatty acyl-CoA to |
| | | forming acyl-CoA | | | fatty alcohol |
| | | reductase) | | | |
| GTNG_1865 | Geobacillustherm | Long-chain aldehyde | YP_001125970 | 1.2.1.3 | reduce fatty aldehydes to |
| | odenitrificans | dehydrogenase | | | fatty alcohols |
| | NG80-2 | | | | |
| AAR | Synechococcus | Acyl-ACP reductase | YP_400611, | 1.2.1.80 | reduce fatty acyl- |
| | elongatus | | WP_011242364 | | ACP/CoA to fatty |
| | | | | | aldehydes |
| carB | Mycobacterium | carboxylic acid | YP_889972, | 1.2.1.30 | reduce fatty acids to fatty |
| | smegmatis | reductase | WP_011730755 | | aldehyde |
| FadD | E. coli K12 | acyl-CoA synthetase | NP_416319 | 6.2.1.3 | activates fatty acids to |
| | | | | | fatty acyl-CoAs |
| atoB | Erwiniacarotovora | acetyl-CoA | YP_049388 | 2.3.1.9 | production of butanol |
| | | acetyltransferase | | | |
| hbd | Butyrivibriofibris | Beta-hydroxybutyryl- | BAD51424 | 1.1.1.157 | production of butanol |
| | olvens | CoA dehydrogenase | | | |
| CPE0095 | Clostridium | crotonasebutyryl- | BAB79801 | 4.2.1.55 | production of butanol |
| | perfringens | CoA dehydryogenase | | | |
| bcd | Clostridium | butyryl-CoA | AAM14583 | 1.3.99.2 | production of butanol |
| | beijerinckii | dehydryogenase | | | |
| ALDH | Clostridium | coenzyme A- | AAT66436 | 1.2.1.3 | production of butanol |
| | beijerinckii | acylating aldehyde | | | |
| | | dehydrogenase | | | |
| AdhE | E. coli CFT073 | aldehyde-alcohol | AAN80172 | 1.1.1.1, | production of butanol |
| | | dehydrogenase | | 1.2.1.10 | |

Fatty Alcohol Acetyl Ester Output

| | | | | | |
|---|---|---|---|---|---|
| | | thioesterases (see | | | modify output |
| | | above) | | | |
| acr1 | Acinetobacter sp. | acyl-CoA reductase | YP_047869 | 1.2.1.42 | modify output |
| | ADP1 | | | | |
| yqhD | E. Coli K12 | alcohol | AP_003562 | 1.1.—.— | modify output |
| | | dehydrogenase | | | |
| AAT | Fragaria × | alcohol O- | AAG13130 | 2.3.1.84 | modify output |
| | ananassa | acetyltransferase | | | |

Terminal Olefin Output

| | | | | | |
|---|---|---|---|---|---|
| OleT | Jeotgalicoccus | Fatty acid | HQ709266 | 1.11.2.4 | decarboxylate fatty acids |
| | sp. | decarboxylase | | | |

Product Export

| | | | | | |
|---|---|---|---|---|---|
| AtMRP5 | Arabidopsis | Arabidopsis thaliana | NP_171908 | none | modify product export |
| | thaliana | multidrug resistance- | | | amount |
| | | associated | | | |
| AmiS2 | Rhodococcus sp. | ABC transporter | JC5491 | none | modify product export |
| | | AmiS2 | | | amount |
| AtPGP1 | Arabidopsis | Arabidopsis thaliana | NP_181228 | none | modify product export |
| | thaliana | p glycoprotein 1 | | | amount |
| AcrA | CandidatusProto- | putative multidrug- | CAF23274 | none | modify product export |
| | chlamydiaamoebophila | efflux transport | | | amount |
| | UWE25 | protein acrA | | | |
| AcrB | CandidatusProto- | probable multidrug- | CAF23275 | none | modify product export |
| | chlamydiaamoebophila | efflux transport | | | amount |
| | UWE25 | protein, acrB | | | |
| TolC | Francisellatularensis | Outer membrane | ABD59001 | none | modify product export |
| | subsp. novicida | protein [Cell | | | amount |
| | | envelope biogenesis, | | | |
| AcrE | Shigellasonnei | transmembrane protein | YP_312213 | none | modify product export |
| | Ss046 | affects septum | | | amount |
| | | formation and cell | | | |
| | | membrane permeability | | | |
| AcrF | E. coli | Acriflavine resistance | P24181 | none | modify product export |
| | | protein F | | | amount |
| tll1619 | Thermosynechococcus | multidrug efflux | NP_682409.1 | none | modify product export |
| | elongatus | transporter | | | amount |
| | [BP-1] | | | | |
| tll0139 | Thermosynechococcus | multidrug efflux | NP_680930.1 | none | modify product export |
| | elongatus | transporter | | | amount |
| | [BP-1] | | | | |

Fermentation

| | | | | | |
|---|---|---|---|---|---|
| replication | | | | | increase output efficiency |
| checkpoint | | | | | |
| genes | | | | | |
| umuD | Shigellasonnei | DNA polymerase V, | YP_310132 | 3.4.21.— | increase output efficiency |
| | Ss046 | subunit | | | |
| umuC | E. coli | DNA polymerase V, | ABC42261 | 2.7.7.7 | increase output efficiency |
| | | subunit | | | |
| pntA, pntB | Shigellaflexneri | NADH: NADPH | P07001, | 1.6.1.2 | increase output efficiency |
| | | transhydrogenase (alpha | P0AB70 | | |
| | | and beta subunits) | | | |

Other

| | | | | | |
|---|---|---|---|---|---|
| fabK | Streptococcus | trans-2-enoyl-ACP | AAF98273 | 1.3.1.9 | Contributes to fatty acid |
| | pneumoniae | reductase II | | | biosynthesis |
| fabL | Bacillus | enoyl-(acyl carrier | AAU39821 | 1.3.1.9 | Contributes to fatty acid |
| | licheniformis | protein) reductase | | | biosynthesis |
| | DSM 13 | | | | |
| fabM | Streptococcus | trans-2, cis-3- | DAA05501 | 4.2.1.17 | Contributes to fatty acid |
| | mutans | decenoyl-ACP | | | biosynthesis |
| | | isomerase | | | |

In some embodiments, a host strain may overexpress one or more of the FAS genes (e.g., any one or more of those described above and/or listed in Table A). Exemplary FAS genes that may be overexpressed include, e.g., FadR from Escherichia coli (see, e.g., GenBank Accession No. NP_415705.1), FabB from Escherichia coli (see, e.g., UniProtKB Accession No. P0A953), or FabZ from Escherichia coli (see, e.g., UniProtKB Accession No. P0A6Q6) or FabZ Acinetobacter baylyi (see, e.g., UniProtKB Accession No. Q6FCG4), as well as homologs thereof and corresponding enzymes, with the same activities, that are derived from other organisms or species. In another embodiment, the host strain encompasses optional overexpression of one or more genes, including, for example, fadR, fabA, fabD, fabG, fabH, fabV, and/or fabF. Examples of such genes are fadR from Escherichia coli, fabA from Salmonella typhimurium (NP_460041), fabD from Salmonella typhimurium (NP_460164), fabG from Salmonella typhimurium (NP_460165), fabH from Salmonella typhimurium (NP_460163), fabV from Vibrio cholera (YP_001217283), and fabF from Clostridium acetobutylicum (NP_350156). In some exemplary embodiments, the overexpression of one or more of these genes, which code for enzymes and regulators in fatty acid biosynthesis, serves to further increase the titer of fatty acids and fatty acid derivative compounds under particular culture conditions.

Also provided herein are cell cultures comprising any of the recombinant cells, microbes, or microorganism, described herein.

### IV. Compositions

Further contemplated herein are fatty acid compositions (or fatty acid derivative compositions) comprising fatty acids and/or derivatives thereof, including, but not limited to, for example, fatty esters, fatty alcohols, fatty alcohol acetates, fatty aldehydes, fatty amines, fatty amides, ω-hydroxy fatty acids, ω-carboxy fatty acids, ω-hydroxy fatty esters, ω-carboxy fatty esters, α,ω-fatty diacids, α,ω-fatty diols, and α,ω-fatty diesters, that are produced by the recombinant cells or microbes (or cell cultures comprising them), or the methods or pathways described herein.

The fatty acid derivative may be a fatty acid methyl ester (FAME), a fatty acid ethyl ester (FAEE), a fatty alcohol, and/or a fatty alcohol acetate. The fatty acid derivative may be purified or otherwise isolated. The fatty acid derivative may be purified or isolated by any appropriate method, including, but not limited to, a two-step centrifugation and water-washing; decanting centrifugation and solvent extraction from a biomass; and whole broth extraction with a water immiscible solvent.

Additionally, the fatty acid (or fatty acid derivative) composition produced by the recombinant cells or microbes, or the cell cultures comprising the recombinant cells or microbes, or the methods or pathways described herein may have a reduced amount of byproducts, such as 3-hydroxy fatty acid derivative byproducts, by comparison to a fatty acid (or fatty acid derivative) composition produced by an otherwise isogenic (recombinant) cell or microbial cell that does not express one or more polypeptides or proteins having (i) R-3-hydroxy acyl-CoA dehydratase activity (EC 4.2.1.134, EC 4.2.1.55), (ii) R-specific enoyl-CoA hydratase activity (EC 4.2.1.119, EC 4.2.1.17), and/or (iii) trans-2-enoyl-CoA reductase activity (EC 1.3.1.44). Additionally or alternatively, the fatty acid (or fatty acid derivative) composition produced by the recombinant cells or microbes, or the cell cultures comprising the recombinant cells or microbes, or the methods or pathways described herein may have a reduced amount of byproducts, such as 3-oxo (and 3-hydroxy) fatty acid derivative byproducts, by comparison to a fatty acid (or fatty acid derivative) composition produced by an otherwise isogenic (recombinant) cell or microbial cell that does not express one or more polypeptides or proteins having (i) 3-oxoacyl-CoA reductase, β-ketoacyl-CoA-reductase, or 3-hydroxy acyl-CoA dehydrogenase activity (EC 1.1.1.35, EC 1.1.1.36, or EC 1.1.1.157); (ii) R-3-hydroxy acyl-CoA dehydratase activity (EC 4.2.1.134, EC 4.2.1.55); (iii) R-specific enoyl-CoA hydratase activity (EC 4.2.1.119, EC 4.2.1.17); and/or (iv) trans-2-enoyl-CoA reductase activity (EC 1.3.1.44). In some embodiments, a “reduced amount or byproducts” correspond to no byproducts, or an undetectable amount of byproducts. For example, in some embodiments, the fatty acid derivative composition is free or substantially free of 3-hydroxy, trans-2-fatty acids, and/or 3-oxo fatty acid and fatty acid derivative byproducts. For example, in some embodiments, the compositions provided herein can contain about 0.01%, 0.1%, 0.2%, 0.3%, 0.4%, 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, less, by weight of the total composition, of 3-OH group containing byproducts (and/or trans-2 fatty acid and trans-2-fatty acid derivative byproducts), compared to a composition produced by a control or reference cell, microbe, or microorganism, that does not contain the R-3-hydroxy acyl-CoA dehydratase, R-specific enoyl-CoA hydratase, and/or trans-2-enoyl-CoA reductase. In some embodiments, the composition can comprise about 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold, less, 3-OH group containing byproducts (and/or trans-2 fatty acid and trans-2-fatty acid derivative byproducts), compared to a composition produced by a control or reference cell, microbe, or microorganism, that does not contain the R-3-hydroxy acyl-CoA dehydratase, R-specific enoyl-CoA hydratase, and/or trans-2-enoyl-CoA reductase. In other embodiments, the compositions provided herein can contain about 0.01%, 0.1%, 0.2%, 0.3%, 0.4%, 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%, less, by weight of the total composition, of 3-oxo and/or 3-hydroxy group containing byproducts (and/or trans-2 fatty acid and trans-2-fatty acid derivative byproducts), compared to a composition produced by a control or reference cell, microbe, or microorganism, that does not contain the 3-oxoacyl-CoA reductase, β-ketoacyl-CoA-reductase, 3-hydroxy acyl-CoA dehydrogenase, R-3-hydroxy acyl-CoA dehydratase, R-specific enoyl-CoA hydratase, and/or trans-2-enoyl-CoA reductase. In some embodiments, the composition can comprise about 1.1-fold, 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold, less, 3-oxo and/or 3-hydroxy group containing byproducts (and/or trans-2 fatty acid and trans-2-fatty acid derivative byproducts), compared to a composition produced by a control or reference cell, microbe, or microorganism, that does not contain the 3-oxoacyl-CoA reductase, β-ketoacyl-CoA-reductase, 3-hydroxy acyl-CoA dehydrogenase, R-3-hydroxy acyl-CoA dehydratase, R-specific enoyl-CoA hydratase, and/or trans-2-enoyl-CoA reductase.

In some embodiments, the compositions provided herein can contain one or more fatty acids and/or derivatives thereof, including, but not limited to, for example, fatty esters, fatty alcohols, fatty alcohol acetates, fatty alcohol acetate esters, fatty aldehydes, fatty amines, fatty amides, ω-hydroxy fatty acids, ω-carboxy fatty acids, ω-hydroxy fatty esters, ω-carboxy fatty esters, α,ω-fatty diacids, α,ω-fatty diols, and α,ω-fatty diesters, and a reduced amount of 3-hydroxy and/or 3-oxo fatty acid and fatty acid derivative byproducts. For example, the compositions can contain less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, less than about 1%, less than about 0.5%, or less of 3-hydroxy and/or 3-oxo fatty acid and/or fatty acid derivative byproducts. In some embodiments, the compositions (or fatty acid derivative compositions) provided herein are free or substantially free of 3-hydroxy, trans-2-fatty acids, and/or 3-oxo fatty acid and/or fatty acid derivative byproducts.

In some embodiments, the target products, i.e., the fatty acids and/or derivatives thereof include one or more of a C6-C20 fatty acid and/or derivative thereof, or a combination thereof. For example, the fatty acids and derivatives thereof can include one or more of hexadecanoic acid, tetradecanoic acid, dodecanoic acid, decanoic acid, octadecanoic acid, or derivatives thereof, e.g., ethyl or methyl ester derivatives thereof. In other embodiments, the fatty acids and derivatives thereof can be monounsaturated and can include one or more of hexadecenoic acid, tetradecenoic acid, dodecenoic acid, decenoic acid, octadecenoic acid, or derivatives thereof, e.g., ethyl or methyl ester derivatives thereof. In other embodiments, the fatty acid derivatives are fatty alcohols or fatty alcohol acetate esters, for example, one or more of hexadecanol, hexadecenol, tetradecanol, tetradecenol, dodecanol, dodecenol, decanol, decenol, octanol, octenol, hexadecanyl acetate ester, hexadecenyl acetate ester, tetradecanyl acetate ester, tetradecenyl acetate ester, dodecanyl acetate ester, dodecenyl acetate ester, decanyl acetate ester, decenyl acetate ester, octanyl acetate ester, and octenyl acetate ester.

In some embodiments, the 3-hydroxy byproducts can include one or more of 3-hydroxy-octanoic acid, 3-hydroxy-decanoic acid, 3-hydroxy-dodecanoic acid, 3-hydroxy-tetradecanoic acid, 3-hydroxy-hexadecanoic acid, 3-hydroxy-octadecanoic acid, 3-hydroxy-octanoic methyl/ethyl ester, 3-hydroxy-decanoic methyl/ethyl ester, 3-hydroxy-dodecanoic methyl/ethyl ester, 3-hydroxy-tetradecanoic methyl/ethyl ester, 3-hydroxy-hexadecanoic methyl/ethyl ester, 3-hydroxy-octadecanoic methyl/ethyl ester, 3-hydroxy-octanol, 3-hydroxy-decanol, 3-hydroxy-dodecanol, 3-hydroxy-tetradecanol, 3-hydroxy-hexadecanol, 3-hydroxy-octadecanol, 3-hydroxy-octanyl acetate ester, 3-hydroxy-decanyl acetate ester, 3-hydroxy-dodecanyl acetate ester, 3-hydroxy-tetradecanyl acetate ester, 3-hydroxy-hexadecanyl acetate ester, and 3-hydroxy-octadecanyl acetate ester, as well as monounsaturated versions thereof.

In some embodiments, the 3-oxo byproducts can include one or more of 3-oxo-octanoic acid, 3-oxo-decanoic acid, 3-oxo-dodecanoic acid, 3-oxo-tetradecanoic acid, 3-oxo-hexadecanoic acid, 3-oxo-octadecanoic acid, 3-oxo-octanoic methyl/ethyl ester, 3-oxo-decanoic methyl/ethyl ester, 3-oxo-dodecanoic methyl/ethyl ester, 3-oxo-tetradecanoic methyl/ethyl ester, 3-oxo-hexadecanoic methyl/ethyl ester, 3-oxo-octadecanoic methyl/ethyl ester, 3-oxo-octanol, 3-oxo-decanol, 3-oxo-dodecanol, 3-oxo-tetradecanol, 3-oxo-hexadecanol, 3-oxo-octadecanol, 3-oxo-octanyl acetate ester, 3-oxo-decanyl acetate ester, 3-oxo-dodecanyl acetate ester, 3-oxo-tetradecanyl acetate ester, 3-oxo-hexadecanyl acetate ester, and 3-oxo-octadecanyl acetate ester, as well as monounsaturated versions thereof.

### V. Methods of Preferentially Producing Fatty Acid Derivatives

Methods for producing fatty acids and derivatives thereof (and fatty acid or fatty acid derivative compositions) and reducing the amount of, or eliminating or substantially eliminating, hydroxylated byproducts, such as 3-hydroxy fatty acids and derivatives thereof, and/or trans-2-fatty acids and derivatives thereof are described herein. Also provided herein are methods for producing fatty acids and derivatives thereof (and fatty acid or fatty acid derivative compositions) and reducing the amount of, or eliminating or substantially eliminating, 3-oxo fatty acid and/or 3-oxo fatty acid derivative byproducts. In some embodiments, the methods result in the reduction, elimination, or substantial elimination of both 3-hydroxy byproducts and 3-oxo byproducts. For example, provided herein are methods for producing a composition comprising fatty acids and/or derivatives thereof, wherein the composition comprises a reduced amount of, or is free or substantially free of, 3-hydroxy, trans-2-fatty acids, and/or 3-oxo fatty acid and/or fatty acid derivative byproducts. In certain embodiments, the method comprises culturing a recombinant cell or microbe, or a cell culture comprising a recombinant cell or microbe, described herein, in the presence of a carbon source. The method may further comprise the step of isolating or otherwise purifying the fatty acid or derivative thereof from the culture or fermentation broth. Additionally, an alcohol (such as methanol or ethanol) may be added to the culture to produce a fatty acid alkyl ester (e.g., fatty acid methyl ester or fatty acid ethyl ester). In a particular embodiment, the fatty acid alkyl ester may be hexadecanoic acid ethyl ester, hecadecanoic acid methyl ester, tetradecanoic acid ethyl ester, tetradecanoic acid methyl ester, dodecanoic acid ethyl ester, dodecanoic acid methyl ester, decanoic acid ethyl ester, decanoic acid methyl ester, octanoic acid methyl ester, octanoic acid ethyl ester, or a combination thereof.

A fatty acid and/or a derivative thereof, as well as a composition comprising a fatty acid and/or a derivative thereof, prepared by the methods described herein, is provided. The fatty acid or derivative thereof may be isolated and/or purified by any known conventional method. For example, by a two-step centrifugation and water-washing; decanting centrifugation and solvent extraction from a biomass; and/or a whole broth extraction with a water immiscible solvent.

### VI. Modified Biosynthetic Pathways

Provided herein are modified biosynthetic pathways, comprising: i) an acyl-ACP thioesterase and an acyl-CoA synthetase; and ii) a heterologous R-3-hydroxy acyl-CoA dehydratase or a heterologous R-specific enoyl-CoA hydratase, wherein the modified biosynthetic pathway produces a fatty acid derivative composition comprising fatty acids and derivatives thereof, and comprising a reduced amount of 3-hydroxy fatty acids and 3-hydroxy fatty acid derivatives, as compared to a corresponding biosynthetic pathway that does not comprise a heterologous R-3-hydroxy acyl-CoA dehydratase or a heterologous R-specific enoyl-CoA hydratase. Also provided herein are modified biosynthetic pathways, comprising: i) an acyl-ACP thioesterase and an acyl-CoA synthetase; ii) a heterologous R-3-hydroxy acyl-CoA dehydratase or a heterologous R-specific enoyl-CoA hydratase; and iii) a heterologous trans-2-enoyl-CoA reductase, wherein the modified biosynthetic pathway produces a fatty acid derivative composition comprising fatty acids and derivatives thereof, and comprising a reduced amount of 3-hydroxy fatty acids and derivatives thereof, as compared to a corresponding biosynthetic pathway that does not comprise a heterologous R-3-hydroxy acyl-CoA dehydratase or a heterologous R-specific enoyl-CoA hydratase, and a heterologous trans-2-enoyl-CoA reductase. In some embodiments, the fatty acid derivative compositions prepared or produced by the modified biosynthetic pathways are free or substantially free of 3-hydroxy fatty acids and derivatives thereof. In some embodiments, the fatty acid derivative compositions prepared or produced by the modified biosynthetic pathways further comprise a reduced amount of trans-2-fatty acids and derivatives thereof, or are free or substantially free of trans-2-fatty acids and derivatives thereof.

Also provided herein is a modified biosynthetic pathway, comprising: i) an acyl-ACP thioesterase and an acyl-CoA synthetase; ii) a heterologous 3-oxoacyl-CoA reductase, a heterologous β-ketoacyl-CoA-reductase, or a heterologous 3-hydroxy acyl-CoA dehydrogenase; and iii) a heterologous R-3-hydroxy acyl-CoA dehydratase or a heterologous R-specific enoyl-CoA hydratase, wherein the modified biosynthetic pathway produces a fatty acid derivative composition comprising fatty acids and derivatives thereof, and comprising a reduced amount of 3-hydroxy fatty acids and 3-hydroxy fatty acid derivatives, and a reduced amount of 3-oxo fatty acids and derivatives thereof, as compared to a corresponding biosynthetic pathway that does not comprise a heterologous 3-oxoacyl-CoA reductase, a heterologous β-ketoacyl-CoA-reductase, or a heterologous 3-hydroxy acyl-CoA dehydrogenase; and/or does not comprise a heterologous R-3-hydroxy acyl-CoA dehydratase or a heterologous R-specific enoyl-CoA hydratase.

In certain embodiments, provided is a modified biosynthetic pathway, comprising: i) an acyl-ACP thioesterase and an acyl-CoA synthetase; ii) a heterologous 3-oxoacyl-CoA reductase, a heterologous-ketoacyl-CoA-reductase, or a heterologous 3-hydroxy acyl-CoA dehydrogenase; iii) a heterologous R-3-hydroxy acyl-CoA dehydratase or a heterologous R-specific enoyl-CoA hydratase; and iv) a heterologous trans-2-enoyl-CoA reductase, wherein the modified biosynthetic pathway produces a fatty acid derivative composition comprising fatty acids and derivatives thereof, and comprising a reduced amount of 3-hydroxy fatty acids and derivatives thereof, and a reduced amount of 3-oxo fatty acids and derivatives thereof, as compared to a corresponding biosynthetic pathway that does not comprise ii), iii), and/or iv). The fatty acid derivative composition can be free or substantially free of 3-hydroxy fatty acids and derivatives thereof and is free or substantially free of 3-oxo fatty acids and derivatives thereof. In some embodiments, the fatty acid derivative composition can further comprise a reduced amount of trans-2-fatty acids and derivatives thereof, or can be free or substantially free of trans-2-fatty acids and derivatives thereof. In some embodiments, the modified biosynthetic pathway is a fatty acid biosynthetic pathway. The recombinant microbes provided herein can comprise any of the modified biosynthetic pathways described herein. Such recombinant microbes can produce a composition comprising fatty acids and derivatives thereof, and comprising a reduced amount of 3-hydroxy fatty acids and derivatives thereof, and a reduced amount of 3-oxo fatty acids and derivatives thereof, as compared to a corresponding microbe that does not comprise the modified biosynthetic pathway.

The fatty acid or derivative thereof, prepared by the modified biosynthetic pathways or the recombinant microbes containing the modified biosynthetic pathways described herein, can be a fatty acid, a fatty ester, a fatty alcohol, a fatty alcohol acetate ester, a fatty aldehyde, a fatty amine, a fatty amide, a fatty diol, a fatty triol, a fatty tetrol, an ω-hydroxy fatty acid, an o-carboxy fatty acid, an ω-hydroxy fatty ester, an ω-carboxy fatty ester, an α,ω-fatty diacid, an α,ω-fatty diester, or an α,ω-fatty diol, or a combination thereof. As described elsewhere herein, the 3-hydroxy fatty acid or derivative thereof can be a 3-hydroxy fatty acid; a 3-hydroxy fatty acid ester; a 1,3-diol; a fatty alcohol 1,3-diacetate (1,3-fatty diol acetate); a 3-hydroxy fatty aldehyde; a 3-hydroxy fatty amine; a 3-hydroxy fatty amide; a fatty diol with a 3-hydroxy group; a fatty triol with a 3-hydroxy group; a fatty tetrol with a 3-hydroxy group; an ω-hydroxy fatty acid with a 3-hydroxy group, an ω-carboxy fatty acid with a 3-hydroxy group, an ω-hydroxy fatty ester with a 3-hydroxy group, an ω-carboxy fatty ester with a 3-hydroxy group, an α,ω-fatty diacid with a 3-hydroxy group, an α,ω-fatty diester with a 3-hydroxy group, or an α,ω-fatty diol with a 3-hydroxy group, or a combination thereof; and/or the 3-oxo fatty acid or derivative thereof can be a 3-oxo fatty acid; a 3-oxo fatty acid ester; a 3-oxo fatty alcohol; a 3-oxo fatty alcohol acetate ester; a 3-oxo fatty aldehyde; a 3-oxo fatty amine; a 3-oxo fatty amide; a 3-oxo fatty diol; a 3-oxo fatty triol; a 3-oxo fatty tetrol; an ω-hydroxy fatty acid with a 3-oxo group, an ω-carboxy fatty acid with a 3-oxo group, an ω-hydroxy fatty ester with a 3-oxo group, an ω-carboxy fatty ester with a 3-oxo group, an α,ω-fatty diacid with a 3-oxo group, an α,ω-fatty diester with a 3-oxo group, or an α,ω-fatty diol with a 3-oxo group, or a combination thereof; and/or the trans-2-fatty acid or derivative thereof can be a trans-2-fatty acid; a trans-2-fatty acid ester; a trans-2-fatty alcohol; a trans-2-fatty alcohol acetate; a trans-2-fatty aldehyde; a trans-2-fatty amine; a trans-2-fatty amide; a trans-2-fatty 1,3-diol; a trans-2-fatty diol; a trans-2-fatty triol; a trans-2-fatty tetrol; a trans-2-ω-hydroxy fatty acid, a trans-2-ω-carboxy fatty acid, a trans-2-o-hydroxy fatty ester; a trans-2-o-carboxy fatty ester; a trans-2-α,ω-fatty diacid; a trans-2-α,ω-fatty diester; or a trans-2-α,o-fatty diol, or a combination thereof.

The modified biosynthetic pathways described herein can produce a fatty acid derivative composition comprising less than 50%, less than 45%, less than 40%, less than 35%, less than 30%, less than 25%, less than 20%, less than 15%, less than 10%, less than 5%, less than 4%, less than 3%, less than 2%, less than 1%, less than 0.5%, or less, by weight of the composition, of i) 3-hydroxy fatty acids and derivatives thereof; ii) trans-2-fatty acids and derivatives thereof; or iii) 3-oxo fatty acids and derivatives thereof; or a combination thereof.

### VII. Uses

The recombinant cell or microbe described herein may be used for a variety of purposes. In a particular embodiment, the recombinant cell or microbe may be used to produce fatty acids and/or derivatives thereof.

Also provided herein are uses of the recombinant microbes, cell cultures, methods, or the modified biosynthetic pathways described herein, for the preparation of a fatty acid derivative, or a fatty acid derivative composition. In some embodiments, provided are uses of the recombinant microbes, cell cultures, methods, or the modified biosynthetic pathways described herein, for the preparation of a fragrance, flavor, pheromone, fuel, nutritional supplement, dietary supplement, pharmaceutical, or nutraceutical, ingredient or product, or a precursor thereof.

The fatty acids and/or derivatives thereof may be prepared by the cultured and/or fermented recombinant cell or microbe, and used in a composition. The fatty acid and/or derivative thereof may be a fermentation product of the recombinant cell or microbe, or of a cell culture comprising the recombinant cell or microbe. Alternatively, the composition may comprise one or more (e.g., two, three, four, five, or more) particular species of fatty acid derivatives. In particular, the composition may be a fragrance, flavor, pheromone, fuel, nutritional supplement, dietary supplement, nutraceutical, or pharmaceutical, or a precursor thereof.

Additionally or alternatively, the fatty acid and/or derivative thereof may be prepared at a time and/or location that is different than when and/or where the composition is prepared. For example, the fatty acid and/or derivative thereof may be produced by the recombinant cell or microbe (or cell culture comprising the same) in one location (e.g., a first facility, city, state, or country), transported to another location (e.g., a second facility, city, state, or country), and incorporated into the composition comprising the fatty acid and/or derivative thereof.

Additionally or alternatively, the fatty acid or derivative thereof may be purified, for example prior to its use in a composition. The fatty acid or derivative thereof may be purified to a purity of at least about 60% free (e.g., at least about 65% free, at least about 70% free, at least about 75% free, at least about 80% free, at least about 85% free, at least about 90% free, at least about 95% free, at least about 96% free, at least about 97% free, at least about 98% free, or at least about 99% free) from other components with which it is associated.

Additionally or alternatively, the fatty acid or derivative thereof may be insoluble or highly insoluble in water. In such cases, the fatty acid or derivative thereof may be in a separate phase from the environment in which the recombinant cell or microbe (or cell culture) resides (e.g., fermentation broth). The fatty acid or derivative thereof may be solid at room temperature. Alternatively, the fatty acid or derivative thereof (e.g., fatty alcohol derivatives) can be a liquid at room temperature.

Additional purification steps may be required, depending on the final product applications and specifications. These steps may include saponification, bleaching, and eventually distillation, if high purity of a single chain length is required. All these are standard unit operations used regularly in the industry.

In particular, purification of the fatty acid or derivatives thereof involves isolating and recovering the fatty acid or derivatives thereof.

Two different approaches may be applied:

One approach includes recovery of the solid phase of biomass plus product via decanting centrifugation, followed by solvent extraction of the product from the biomass with an appropriate solvent (i.e., methanol or ethanol). The fatty acids and derivatives thereof dissolve in the solvent and the biomass is removed either by centrifugation or filtration. The recovery of the fatty acids and derivatives thereof is then completed by evaporating the solvent. Depending on the application, the product can be further used as a solution in the solvent, or as a solid. Other purification steps, including distillation, can be applied to meet final specifications.

Another approach includes recovery of the product via whole broth extraction with a water immiscible solvent. In this approach, the fermentation broth is contacted in either batch or continuous schemes with an appropriate solvent (i.e., butyl acetate, medium chain alcohols or esters) to allow for the complete dissolution of the product in the solvent. The light organic solvent phase can be separated from the water phase in a similar way as those described for the recovery of the long chain alcohols. Once a clear solvent phase has been obtained, the final product is again recovered by solvent evaporation.

Additionally or alternatively, the fatty acid and/or derivative thereof may be prepared by the recombinant cell or microbe (or cell culture comprising the recombinant cell or microbe), or a composition comprising the fatty acid and/or derivative thereof may be prepared by the recombinant cell or microbe (or cell culture comprising the recombinant cell or microbe), which is incorporated into a product. This product may be made by combining, mixing, or otherwise using the fatty acid and/or derivative thereof produced by the recombinant cell or microbe (or cell culture), in combination with other or more additional components, to prepare the product. The product may comprise one or more (e.g., two, three, four, five, or more) fatty acids and/or derivatives thereof prepared by the recombinant cell or microbe or cell culture. In a particular embodiment, the product is a pheromone or a precursor thereof, a fragrance or a precursor thereof, a pharmaceutical agent or a precursor thereof, a flavor or a precursor thereof, a nutritional/dietary supplement or a precursor thereof, or a nutraceutical or a precursor thereof.

Thus, also provided herein are uses of the fatty acids and derivatives thereof, and uses of the compositions, such as the fatty acid derivative compositions, for the preparation of a fragrance, flavor, pheromone, fuel, nutritional supplement, dietary supplement, pharmaceutical, or nutraceutical, ingredient or product, or a precursor thereof.

Also provided herein is a fragrance, flavor, pheromone, fuel, nutritional supplement, dietary supplement, pharmaceutical, or nutraceutical, ingredient or product, or a precursor thereof, prepared by any of the recombinant microbes, cell cultures, methods and/or modified biosynthetic pathways described herein. The fragrance, flavor, pheromone, fuel, nutritional supplement, dietary supplement, pharmaceutical, or nutraceutical, ingredient or product, or a precursor thereof, comprises the fatty acids or derivatives thereof, or compositions, such as the fatty acid derivative compositions, described herein.

### EXAMPLES

The following examples are provided to further illustrate the invention disclosed herein but should not be construed as in any way limiting its scope.

As discussed above (and shown, for example, in FIG. 1), unwanted fatty acid species byproducts, namely those containing a 3-hydroxy (3-OH) group (i.e., 3-OH group containing fatty acids and derivatives thereof, collectively referred to herein as “3-OH byproducts” or “3-OH fatty acid species byproducts”) are produced by the native acyl-ACP dependent fatty acid derivative biosynthetic pathway. This is due to the formation and accumulation of 3-hydroxy-acyl-ACP (3-OH-acyl-ACP) intermediates (in addition to acyl-ACP intermediates), and the subsequent conversion of the 3-OH-acyl-ACPs to 3-hydroxy fatty acids and derivatives thereof by various different fatty acid derivative enzymes. For example, native/endogenous thioesterases, which can be promiscuous, can convert the 3-OH-acyl-ACPs to the corresponding 3-OH fatty acids. The 3-OH fatty acids can be converted to 3-OH-acyl-CoAs by an acyl-CoA synthetase, and the 3-OH-acyl-CoAs can then be converted to 3-OH fatty esters by ester synthases, or to 1,3-fatty diols by the combination of an acyl-CoA reductase and an alcohol dehydrogenase (see, e.g., FIG. 1). Alternatively, the 3-OH-acyl-CoAs can be converted to 3-OH fatty aldehydes by an acyl-CoA reductase, or can be converted to 1,3-fatty diols by a fatty alcohol forming acyl-CoA reductase. The 1,3-fatty diols can further be converted to fatty alcohol 1,3-diacetates by the action of an alcohol acetyl-CoA transferase, for example (see, e.g., FIG. 1). Alternatively, the 3-OH fatty acids can be reduced to 3-OH fatty aldehydes by a carboxylic acid reductase; the 3-OH fatty aldehydes can be reduced to 1,3-fatty diols by an alcohol dehydrogenase; and the 1,3-fatty diols can further be converted to fatty alcohol 1,3-diacetates by the action of an alcohol acetyl-CoA transferase. The 3-OH fatty aldehydes can be converted to 3-OH fatty amines by a transaminase or aminotransferase. The same fatty acid derivative enzymes act on the corresponding intermediates lacking the 3-OH group, to produce fatty acids and derivatives thereof without a 3-OH group. Other fatty acid derivative enzymes can produce other fatty acid derivatives (e.g., fatty amines, fatty amides, omega-hydroxy fatty acids, omega-hydroxy fatty esters, etc.), as described elsewhere herein, and as known in the art. For example, an omega-hydroxylase (ω-hydroxylase) can add a hydroxy group to the omega (@) position of a fatty acid, a 3-OH fatty acid, a fatty ester, a 3-OH fatty ester, a fatty alcohol, or a 1,3-fatty alcohol. Additionally or alternatively, the aldehyde group on a fatty aldehyde or a 3-OH fatty aldehyde, or on a bifunctional or multifunctional fatty acid derivative (such as, e.g., an omega-carboxy fatty acid; an omega-carboxy, 3-hydroxy-fatty acid; an omega-carboxy fatty ester, an omega-carboxy,3-hydroxy fatty ester) can be converted to an amine group by an aminotransferase/transaminase, or to a carboxylic acid group by an aldehyde dehydrogenase/oxidase (see, e.g., WO 2014/201474 and WO 2019/217226, each of which is incorporated herein by reference in its entirety).

As shown in FIG. 1, and described elsewhere herein, the 3-OH-acyl-ACP intermediates can also be converted to trans-2-enoyl-ACP by the action of enzymes having 3-hydroxy-acyl-ACP dehydratase activity (e.g., FabA and/or FabZ), and the trans-2-enoyl-ACP is converted to acyl-ACP by enzymes having enoyl-ACP reductase activity (e.g., FabI, FabL, FabV). The acyl-ACPs, which do not contain a 3-OH group, can then be converted to the corresponding fatty acids and derivatives thereof by the appropriate fatty acid derivative enzyme(s).

As shown in FIG. 2, the production of 3-OH-containing fatty acids and derivatives thereof (i.e., 3-OH fatty acid species or 3-OH byproducts) can be decreased (or eliminated or substantially eliminated) by converting 3-hydroxy-acyl-CoAs to the corresponding trans-2-enoyl-CoAs by the activity of an enoyl-CoA hydratase (e.g., an R-specific enoyl-CoA hydratase) or by a 3-hydroxy-acyl-CoA dehydratase (e.g., an R-3-hydroxy-acyl-CoA dehydratase). The trans-2-enoyl-CoAs can then be converted to the corresponding acyl-CoAs (not containing a 3-OH group) by an enzyme having trans-2-enoyl-CoA reductase, and the acyl-CoAs can be converted to different fatty acid derivatives not containing a 3-OH group, by the appropriate fatty acid derivative enzymes. For example, the acyl-CoAs can be converted to fatty acid esters by an ester synthase; or to fatty aldehydes by an acyl-CoA reductase; or to fatty alcohols by an acyl-CoA reductase and an alcohol dehydrogenase or by a fatty alcohol forming acyl-CoA reductase; or to fatty alcohol acetates by an acyl-CoA reductase, an alcohol dehydrogenase, and an alcohol acetyl-CoA transferase, or by a fatty alcohol forming acyl-CoA reductase and an alcohol acetyl-CoA transferase, etc. (see, e.g., FIG. 2). The acyl-CoAs can also be converted to free fatty acids by a thioesterase (e.g., an acyl-CoA thioesterase), and the fatty acids can then be converted to different fatty acid derivatives by the appropriate fatty acid derivative enzymes, for example, to fatty aldehydes by a carboxylic acid reductase (CAR); to fatty alcohols by a CAR and an alcohol dehydrogenase (ADH); to fatty amines by a CAR and a transaminase; to omega-hydroxy fatty acids by an omega-hydroxylase; to α,ω-diols by a CAR, ADH, and omega-hydroxylase, etc. (see, e.g., WO 2014/201474 and WO 2019/217226, each of which is incorporated herein by reference in its entirety).

As shown in FIG. 3 and described elsewhere herein, the acyl-ACP dependent fatty acid biosynthetic pathway can also result in the production of unwanted byproducts containing a 3-oxo group. These 3-oxo byproducts result from the conversion of 3-keto-acyl-ACP intermediates of the acyl-ACP dependent fatty acid biosynthetic pathway to the corresponding 3-oxo fatty acids by a thioesterase. The 3-oxo fatty acids can then be converted to 3-oxo-acyl-CoA intermediates by an acyl-CoA synthetase. The 3-oxo fatty acids and 3-oxo-acyl-CoAs result in the generation of unwanted 3-oxo fatty acid derivatives, as described herein, such as for example, 3-oxo fatty esters, 3-oxo fatty alcohols, 3-oxo fatty alcohol acetate esters, 3-oxo fatty aldehydes, etc. The expression of a 3-oxoacyl-CoA reductase, β-ketoacyl-CoA-reductase or 3-hydroxy acyl-CoA dehydrogenase converts 3-oxo-acyl-CoA to 3-hydroxy-acyl-CoA, and the 3-hydroxy-acyl-CoA can then be converted to trans-2-enoyl-CoA as described above for FIG. 2, i.e., by the expression of a an enoyl-CoA hydratase (e.g., an R-specific enoyl-CoA hydratase) or a 3-hydroxy-acyl-CoA dehydratase (e.g., an R-3-hydroxy-acyl-CoA dehydratase). The trans-2-enoyl-CoAs can then be converted to the corresponding acyl-CoAs by an enzyme having trans-2-enoyl-CoA reductase, and the acyl-CoAs can be converted to different fatty acid derivatives not containing a 3-OH or 3-oxo group, by the appropriate fatty acid derivative enzymes. Thus, the pathway comprising i) a 3-oxoacyl-CoA reductase, β-ketoacyl-CoA-reductase or 3-hydroxy acyl-CoA dehydrogenase; ii) an R-specific enoyl-CoA hydratase or R-3-hydroxy-acyl-CoA dehydratase; and iii) optionally a trans-2-enoyl-CoA reductase (see, e.g., FIG. 3), reduces, eliminates, or substantially eliminates the production of both 3-oxo and 3-hydroxy byproducts, since both the 3-hydroxy fatty acids and 3-oxo fatty acids are converted to 3-hydroxy-acyl-CoA intermediates.

Thus, in order to increase the amount (e.g., titer, yield, and/or productivity) of the target or desired fatty acids and/or fatty acid derivative products, i.e., fatty acid species not containing a 3-hydroxy group, and/or to decrease the amount of (e.g., titer, yield, and/or productivity of), or to eliminate or substantially eliminate the production of, the corresponding 3-hydroxy versions of the fatty acids and/or derivatives thereof, i.e., the 3-OH byproducts, recombinant microbes that express an R-specific enoyl-CoA hydratase, with or without a trans-2-enoyl-CoA reductase, were generated and cultured, and the fatty acid species produced by the recombinant strains were analyzed, and compared to the fatty acid species produced by control strains that did not express an R-specific enoyl-CoA hydratase or a trans-2-enoyl-CoA reductase. Examples 2-6 below describe exemplary strains engineered to produce compositions with reduced amounts of 3-OH fatty acid species byproducts. The particular fatty acid derivatives produced by the recombinant strains described below are exemplary; the expression of the R-specific enoyl-CoA hydratase and/or trans-2-enoyl-CoA reductase results in the increased production of acyl-thioesters (acyl-CoAs) without a 3-OH group. The acyl-CoAs can be converted to fatty acid derivatives other than fatty esters, fatty alcohols, or fatty alcohol acetates, by the action of the appropriate endogenous/native, or heterologous fatty acid derivative enzymes, as described above and elsewhere herein.

To increase the amount (e.g., titer, yield, and/or productivity) of the target or desired fatty acids and/or fatty acid derivative products, i.e., fatty acid species not containing a 3-hydroxy and/or 3-oxo group, and/or to decrease the amount of (e.g., titer, yield, and/or productivity of), or to eliminate or substantially eliminate the production of, the corresponding 3-hydroxy and/or 3-oxo versions of the fatty acids and/or derivatives thereof, i.e., the 3-OH and/or 3-oxo byproducts, recombinant microbes that express i) a 3-oxoacyl-CoA reductase, β-ketoacyl-CoA-reductase or 3-hydroxy acyl-CoA dehydrogenase; and ii) an R-specific enoyl-CoA hydratase or R-3-hydroxy-acyl-CoA dehydratase; and iii) optionally a trans-2-enoyl-CoA reductase are describes in Example 7 below. As discussed above, the particular fatty acid derivatives produced by the recombinant strains described below are exemplary, and other fatty acid derivatives can be produced by the same strains as described elsewhere herein, and as known in the art.

### Example 1: General Protocols

### Small Scale Fermentation

A 40 μL LB culture (from an LB culture growing in a 96 well plate) was used to inoculate 360 μL of LB media which was then incubated for approximately 4 hours at 32° C. with shaking. 80 μL of the LB seed was used to inoculate 320 μL of N-LIM media (see, Table 3), optionally containing alcohol (either methanol or ethanol; 2% v/v) when the strains were engineered to produce fatty esters. After growing at 32° C. for 2 hours, the cultures were induced with IPTG (final concentration 1 mM). The cultures were then incubated at 32° C. with shaking for 20 hours (unless noted otherwise), after which they were extracted following the standard extraction protocol detailed below.

TABLE 3
N-LIM Media Formulation
N-LIM Media Formulation

| | | |
|---|---|---|
| 1 | x | 5x Salt Soln. with NH₄Cl |
| 1 | x | 1000x Trace Vitamins |
| 1 | mg/L | 10 mg/mL Thiamine |
| 1 | mM | 1M MgSO₄ |
| 0.1 | mM | 1M CaCl₂ |
| 40 | g/L | 500 g/L glucose |
| 1 | x | 1000x Trace minerals |
| 10 | mg/L | 10 g/L Fe Citrate |
| 100 | μg/mL | 100 mg/ml spectinomycin |
| 100 | mM | 2M BisTris (pH 7.0) |
| 0.5 | mM | Aminolevulinic acid |

### Fatty Acid Species Standard Extraction and Analytical Protocols

To each well to be extracted, 80 μL of 1M HCl, followed by 400 μL of butyl acetate, containing 500 mg/L 1-undecanol or 500 mg/L undecanoic acid as internal standard (IS), was added. The 96 well plates were then heat-sealed and shaken for 15 minutes at 2000 rpm. After shaking, the plates were centrifuged for 10 minutes at 4500 rpm at room temperature to separate the aqueous and organic layers. 50 μL of the organic layer was transferred to a 96 well plate and derivatized with 50 μL of trimethylsiloxy/N,O-Bis(trimethylsilyl) trifluoroacetamide (TMS/BSTFA). The plate was subsequently heat-sealed and stored at −20° C. until the sample was evaluated by either Gas Chromatography with Flame Ionization Detection (GC-FID) or Gas Chromatography-Mass Spectrometry (GC-MS).

### Fatty Acid Species Analytics:

The GC-MS parameters used to generate chromatograms and mass spectra for compound identification were as follows:

TABLE 4
GC-MS Parameters

| Sample volume | 1 | μL |
|---|---|---|
| Column | DB-1HT, 15 m × 250 μm × 0.1 μm | |
| Initial temperature | 50° C. for 5 minutes | |
| Final Temperature | 300° C., held for 5.24 minutes | |
| Temperature increase rate | 25° | C./minute |
| Total run time | 24 | minutes |
| Column flow rate | 1.2 | mL/min |
| Inlet temperature | 300° | C. |

| | |
|---|---|
| Split ratio | 20:1 |
| Analyzing software | ChemStation E.02.01.1177 |

The mass spectrometry parameters are shown in Table 5.

TABLE 5
Mass Spectrometry Parameters

| | |
|---|---|
| Transfer line temperature | 300° C. |
| MS source temperature | 230° C. |
| MS Quad temperature | 150° C. |
| Autosampler | CombiPAL (CTC analytics) |

The GC-FID parameters used to quantify each compound are shown in Table 6:

TABLE 6
GC-FID Parameters

| Sample volume | 1 | μL |
|---|---|---|
| Column | UFC Rtx-1, 5M × 0.1 mm × 0.1 μM | |
| | (Thermo Fisher Ultrafast TRACE GC) | |
| Initial oven temperature | 100° C. (0.2 minutes) | |
| Final oven temperature | 320° C. (0.5 minutes) | |
| Temperature increase rate | 100° | C./min |
| Total run time | 2.5 | minutes |
| Column flow rate | 0.5 | mL/min |
| Inlet temperature | 300° | C. |
| Flame ionization detector | 300° | C. |
| temperature | | |

The protocols detailed above represent standard conditions, and a person having ordinary skill in the art appreciates that the protocol may be modified as necessary, for example, to optimize the analytical results.

### Example 2: Production of Fatty Acid Derivatives with Reduced 3-Hydroxy Byproducts by Recombinant Strains Expressing a Heterologous R-Specific Enoyl-CoA Hydratase

This Example illustrates the production of a fatty acid derivative composition with a reduced amount of 3-hydroxy (3-OH) fatty acid derivative byproducts. The fatty acid derivative composition is exemplified herein by a fatty ester composition, particularly, a fatty acid methyl ester (FAME) composition, with a reduced amount of 3-OH FAME byproducts. This was achieved herein by engineering (or genetically modifying) bacterial strains (exemplified herein by E. coli) to express various different heterologous R-specific enoyl-CoA hydratases (e.g., MaoC family dehydratases).

An E. coli MG1655 derivative strain was engineered to lack (i.e., knockout or delete) acyl-CoA dehydrogenase (FadE) (SEQ ID NO: 26) and to overexpress acyl-CoA synthetase (FadD) (SEQ ID NO: 27). As described elsewhere herein, FadE attenuation or deletion is optional. A plasmid derived from a pCL1920-derivative vector, containing an SC101 replicon, a spectinomycin resistance marker, and nucleic acid sequences encoding (i) an acyl-ACP thioesterase (FatB1) from Umbellularia californica (UniProtKB Accession No. Q41635) (SEQ ID NO: 28), (ii) an ester synthase from Limnobacter (UniProtKB Accession No. Δ6GSQ9) (SEQ ID NO: 29), (iii) a B-keto-acyl-ACP synthase I from E. coli (FabB; UniProtKB Accession No. POA953) (SEQ ID NO: 30), and (iv) a transcriptional regulator (FadR from E. coli) (SEQ ID NO: 31; GenBank Accession No. WP_000234823), all placed under the control of an IPTG-inducible Ptrc promoter, was transformed into the engineered E. coli strain to produce control strain sAS.410.

Four different R-specific enoyl-CoA hydratase (phaJ) genes were each separately cloned into a pACYC-derivative vector (comprising a p15A replicon and a kanamycin resistance marker): phaJ1 from Pseudomonas putida (“PhaJ1_P.put”) (encoding SEQ ID NO: 1); phaJ4 from P. putida (“PhaJ4_P.put”) (encoding SEQ ID NO: 2); phaJ3 from P. aeruginosa (“PhaJ3_P.aer”) (encoding SEQ ID NO: 3); and phaJ4 from P. aeruginosa (“PhaJ4_P.aer”) (encoding SEQ ID NO: 4). The R-specific enoyl-CoA hydratase genes were placed under the control of an IPTG-inducible Ptrc promoter and transformed into the sAS.410 strain described above, to produce four strains that each expressed a heterologous R-specific enoyl-CoA hydratase, namely sAS.411 (expressing PhaJ1_P.put), sAS.412 (expressing PhaJ4_P.put), SAS.413 (expressing PhaJ4_P.aer), and sAS.414 (expressing PhaJ3_P.aer).

Each of strains sAS.411, sAS.412, sAS.413, and sAS.414, and control strain sAS.410, were subjected to small scale fermentation in the presence of methanol (see, Example 1), and product (fatty acid species) analysis was performed as described in Example 1. All strains produced fatty acid methyl esters (FAME), 3-hydroxy fatty acid methyl esters (3-OH FAME), free fatty acids (FFA), and 3-hydroxy free fatty acids (3-OH FFA). The predominant chain length of the fatty acid species produced was C12 (due to the specificity/selectivity of the FatB1 thioesterase), and the most abundant product was dodecanoic acid methyl ester.

The composition of FAME and 3-OH FAME produced by each of the five strains described above is shown in Table 7 below. The titer of total FAME, which includes both FAME without a 3-hydroxy (3-OH) group and 3-OH FAME (byproduct), produced by each strain, is provided, as well as the titer of 3-OH FAME, and the percentage of the total FAME titer that corresponded to 3-OH FAME. As shown in Table 7, all strains expressing a PhaJ R-specific enoyl-CoA hydratase produced more total FAME (i.e., a higher titer), and less 3-OH FAME, as compared to control strain sAS.410, which did not express an R-specific enoyl-CoA hydratase. PhaJ4 from P. putida (“PhaJ4_P.put”), as well as PhaJ3 and PhaJ4 from P. aeruginosa (“PhaJ3_P.aer” and “PhaJ4_P.aer”, respectively), were most effective at reducing the amount (both titer and percentage) of 3-OH FAME byproducts. These results indicate that the expression of an R-specific enoyl-CoA hydratase, which converts 3-hydroxy-acyl-CoAs to trans-2-enoyl-CoAs, decreases the amount of 3-hydroxy group containing fatty acid species (i.e., byproducts) produced.

All strains that expressed an R-specific enoyl-CoA hydratase also produced small amounts of trans-2-unsaturated FAMEs (e.g., trans-2-dodecenoic acid methyl ester; data not shown). This indicates that a small amount of the trans-2-enoyl-CoA intermediates, generated by the activity of the R-specific enoyl-CoA hydratase, were directly converted to the corresponding trans-2 fatty esters by the activity of the encoded ester synthase, instead of being converted to the corresponding acyl-CoAs first. This, in turn, indicates that the trans-2-enoyl-CoA intermediates accumulated at a rate faster than their conversion to acyl-CoAs, indicating that, in these strains, trans-2-enoyl-CoA reductase activity was limiting.

TABLE 7
Fatty Acid Derivative Compositions Produced by Recombinant Strains
with and without R-specific Enoyl-CoA Hydratase Expression.

| | | Total FAME | Total 3-OH | % of |
|---|---|---|---|---|
| | Enoyl-CoA | Titer* | FAME Titer | 3-OH |
| Strain | Hydratase | (mg/L) | (mg/L) | FAME |
| sAS.410 | none | 797 | 289 | 36 |
| (Control) | | | | |
| sAS.411 | PhaJ1_P.put | 835 | 183 | 22 |
| sAS.412 | PhaJ4_P.put | 849 | 32 | 4 |
| pAS.413 | PhaJ4_P.aer | 807 | 39 | 5 |
| sAS.414 | PhaJ3_P.aer | 872 | 31 | 4 |

*Total FAME Titer includes FAME without a 3-OH group and 3-OH FAME.

This example demonstrates that expression of an enoyl-CoA hydratase reduces 3-hydroxy fatty acid ester byproducts in recombinant strains that express an acyl-ACP dependent fatty acid biosynthetic pathway.

The results also demonstrate that four different enoyl-CoA hydratases, from two different species, having amino acid sequences with percent identities ranging from approximately 29% to approximately 78%, are all capable of producing fatty acid derivative compositions with reduced amounts (e.g., titers and percentages) of 3-hydroxy fatty acid derivative byproducts. For example, alignments of the sequences of PhaJ1_P.put (SEQ ID NO: 1), PhaJ4_P.put (SEQ ID NO:2), PhaJ3_P.aer (SEQ ID NO:3), and PhaJ4_P.aer (SEQ ID NO: 4), using BLASTP, resulted in the following sequence identities:

| | PhaJ1_P.put | PhaJ4_P.put | PhaJ3_P.aer |
|---|---|---|---|
| PhaJ1_P.put | N/A | 31.53% | 39.68% |
| PhaJ4_P.put | 31.53% | N/A | 38.71% |
| PhaJ3_P.aer | 39.68% | 38.71% | N/A |
| PhaJ4_P.aer | 30.77% | 78.15% | 29.00% |

Due to the low percent identities between the amino acid sequences of the four different enoyl-CoA hydratases utilized in this Example, it is expected that other homologs of PhaJ or other enoyl-CoA reductases, from a wide variety of species, that can catalyze the conversion of 3-OH acyl-CoA to the corresponding trans-2-enoyl-CoA, can also be expressed in the recombinant cells and microbes provided herein, to produce fatty acid derivative compositions with reduced amounts 3-hydroxy fatty acid derivative byproducts.

The production of FAME is exemplary and was achieved by fermentation/culturing in the presence of methanol; other fatty acid ester derivatives can readily be produced by the strains described herein, by the addition of the appropriate alcohol to the culture media. For example, fatty acid ethyl esters can be produced by growing the strains in the presence of ethanol instead of methanol (see, e.g., Example 4 below), or fatty acid propyl esters can be produced by growing the strains in the presence of propanol, and so forth.

This Example describes recombinant cells/microbes that produce fatty acid esters by engineering them to express an ester synthase. The production of fatty esters, however, also is exemplary. Additionally, or alternatively, other fatty acid derivatives can be produced by the strains described herein by the activities of other fatty acid derivative enzymes. Such fatty acid derivative enzymes can be native or endogenous to the recombinant cells/microbes, and can be optionally overexpressed, or they can be heterologous (i.e., derived from another species), and can be expressed or overexpressed in any of the recombinant cells, microbes, or strains described herein. For example, free fatty acids can be produced when a thioesterase (e.g., an acyl-CoA thioesterase) is expressed; fatty alcohols can be produced when one or more fatty alcohol biosynthetic polypeptides are expressed (e.g., a carboxylic acid reductase (CAR) or an acyl-ACP reductase (AAR) and an alcohol dehydrogenase (ADH), or a fatty alcohol-forming acyl-CoA reductase (FAR)); fatty aldehydes can be produced when one or more fatty aldehyde biosynthetic polypeptides are expressed (e.g., a CAR or an AAR); fatty amines can be produced when a transaminase and one or more fatty aldehyde biosynthetic polypeptides are expressed; an omega-hydroxy fatty acid can be produced when a thioesterase and an omega-hydroxylase are expressed, etc.

### Example 3: Production of Fatty Acid Derivatives with Reduced 3-Hydroxy Byproducts by Recombinant Strains Expressing a Heterologous Enoyl-CoA Hydratase and a Heterologous Trans-2-Enoyl-CoA Reductase

As discussed above (see, Example 2), recombinant strains expressing a heterologous enoyl-CoA hydratase were capable of reducing the amount of 3-hydroxy fatty acid derivative byproducts, indicating that native, endogenous enzymes in these strains can convert trans-2-enoyl-CoA to acyl-CoA. However, the strains also produced small amounts of trans-2-fatty acid derivatives from the trans-2-enoyl-CoA intermediates, indicating that the activity of trans-2-enoyl-CoA reductase was limiting. To determine whether the expression of a heterologous trans-2-enoyl-CoA reductase, in addition to the enoyl-CoA-hydratase, further reduces the amount of 3-OH fatty acid derivatives produced, recombinant strains that expressed both enzymes were generated and cultured, and the compositions of fatty acid species produced were analyzed.

This Example illustrates the production of a fatty acid derivative composition with a reduced amount of 3-hydroxy (3-OH) fatty acid derivative byproducts, by recombinant microorganisms engineered to express a heterologous enoyl-CoA hydratase and a heterologous trans-2-enoyl-CoA reductase. The fatty acid derivative composition is exemplified herein by a fatty ester composition, specifically, a fatty acid methyl ester (FAME) composition, with a reduced amount of 3-OH FAME byproducts. However, as described herein, other types of esters (e.g., FAEEs), can be produced by adjusting the fermentation/culture conditions (e.g., by adding ethanol instead of methanol; see, e.g., Example 4 below), and other fatty acid derivatives can readily be produced by expressing the appropriate fatty acid derivative enzymes (see, e.g., Examples 5 and 6 below).

Specifically, this Example demonstrates the production of a fatty acid methyl ester (FAME) composition, with a reduced amount of 3-OH FAME byproduct, by engineering bacterial strains (exemplified herein by E. coli) to express a heterologous R-specific enoyl-CoA hydratase and a heterologous trans-2-enoyl-CoA reductase.

An E. coli MG1655 derivative strain (was engineered to lack (i.e., knockout or delete) acyl-CoA dehydrogenase (FadE; SEQ ID NO:26) and to overexpress acyl-CoA synthetase (FadD; SEQ ID NO:27). This base strain was designated stEP.957. As described elsewhere herein, FadE attenuation or deletion is optional. A plasmid derived from a pCL1920-derivative vector, containing an SC101 replicon, a spectinomycin resistance marker, and nucleic acid sequences encoding (i) an acyl-ACP thioesterase (FatB1) from Umbellularia californica (UniProtKB Accession No. Q41635; SEQ ID NO:28), (ii) an ester synthase from Limnobacter (UniProtKB Accession No. A6GSQ9; SEQ ID NO:29), (iii) a β-keto-acyl-ACP synthase I from E. coli (FabB, UniProtKB Accession No. POA953; SEQ ID NO:30), and (iv) a transcriptional regulator (FadR from E. coli; SEQ ID NO:31), all placed under the control of an IPTG-inducible Ptrc promoter, was transformed into the engineered E. coli strain to produce control strain stEP.973.

The R-specific enoyl-CoA hydratase genes phaJ4 from P. putida (“PhaJ4_P.put”), and phaJ4 from P. aeruginosa (“PhaJ4_P.aer”) each were separately cloned into a pCL1920-derivative vector (comprising an SC101 replicon and a spectinomycin resistance marker), with or without the trans-2-enoyl-CoA reductase gene ter (or fabV) from Treponema denticola (UniProtKB Accession No. Q73Q47; SEQ ID NO:14; “TER_T.den”). The R-specific enoyl-CoA hydratase gene, and, where applicable, the trans-2-enoyl-CoA reductase gene, were placed under the control of an IPTG-inducible PT5 promoter, and each of the vectors was transformed into the stEP.973 strain to produce strains that express a heterologous R-specific enoyl-CoA hydratase, (sAS.437 and sAS.419), and strains that express both a heterologous R-specific enoyl-CoA hydratase and a trans-2-enoyl-CoA reductase (sAS.444 and sAS.445). As summarized in Table 8 below, control strain stEP.973 did not contain an enoyl-CoA hydratase or a trans-2-enoyl-CoA reductase; strain sAS.437 contained the enoyl-CoA hydratase PhaJ4 from P. putida; strain sAS.419 contained the enoyl-CoA hydratase PhaJ4 from P. aeruginosa; strain sAS.444 contained the enoyl-CoA hydratase PhaJ4 from P. aeruginosa and the trans-2-enoyl-CoA reductase from T. denticola; and strain sAS.445 contained the enoyl-CoA hydratase PhaJ4 from P. putida and the trans-2-enoyl-CoA reductase from T. denticola.

Each of strains sAS.419, sAS.437, sAS.444, and sAS.445, and control strain (stEP.973), were subjected to small scale fermentation in the presence of methanol and product analysis as described in Example 1. All strains produced fatty acid methyl esters (FAME). The predominant chain length of the fatty acid derivatives was C12, and the most abundant product was dodecanoic acid methyl ester. The composition of FAME and 3-OH FAME produced by each of the five strains described above is shown in Table 8. The titer of total FAME, which includes both FAME without a 3-hydroxy (3-OH) group and 3-OH FAME byproduct, produced by each strain, is provided, as well as the titer of 3-OH FAME byproduct, and the percentage of the total FAME titer that corresponds to 3-OH FAME byproduct.

As shown in Table 8, the two strains that expressed an R-specific enoyl-CoA hydratase (but did not express a trans-2-enoyl-CoA reductase) (i.e., strains sAS.419 and sAS.437) produced a decreased amount (titer and percentage of total FAME) of 3-OH FAME byproduct relative to the control stEP.973 strain. The two strains that expressed an R-specific enoyl-CoA hydratase together with a trans-2-enoyl-CoA reductase (i.e., strains sAS.444 and sAS.445) did not produce any detectable 3-OH FAME byproduct and still maintained high FAME productivity (titer). These results indicate that the expression of a heterologous trans-2-enoyl-CoA reductase, in addition to a heterologous enoyl-CoA hydratase, further decreases or eliminates (or substantially eliminates) the amount of 3-hydroxy fatty acid species (i.e., byproducts) produced.

Strains sAS.444 and sAS.445 also did not produce detectable levels of trans-2-unsaturated fatty acid derivatives (exemplified herein by trans-2-unsaturated FAMEs) (data not shown), indicating that, in these strains, trans-2-enoyl-CoA reductase activity was no longer limiting.

TABLE 8
Fatty Acid Derivative Compositions Produced byRecombinant
Strains with and without R-specific Enoyl-CoA Hydratase
and Trans-2-enoyl-CoA Reductase Expression.

| | | | | Total | |
|---|---|---|---|---|---|
| | | Trans-2- | Total | 3-OH FAME | 3-OH FAME |
| | Enoyl-CoA | Enoyl-CoA | FAME | (Byproduct) | (Byproduct) |
| Strain | Hydratase | Reductase | Titer* (mg/L) | Titer (mg/L) | % |
| stEP.973 | none | none | 1660 | 262 | 15.8 |
| (control) | | | | | |
| sAS.437 | PhaJ4_P.put | none | 1575 | 72 | 4.6 |
| sAS.419 | PhaJ4_P.aer | none | 1836 | 60 | 3.3 |
| sAS.445 | PhaJ4_P.put | TER_T.den | 1376 | 0 | 0.0 |
| sAS.444 | PhaJ4_P.aer | TER_T.den | 1142 | 0 | 0.0 |

*Total FAME Titer includes FAME without a 3-OH group and 3-OH FAME byproduct.

This example demonstrates that expression of an R-specific enoyl-CoA hydratase and a trans-2-enoyl-CoA reductase significantly reduces or eliminates (or substantially eliminates) 3-hydroxy fatty acid ester byproducts produced by recombinant strains that expresses an acyl-ACP dependent fatty acid biosynthetic pathway for the production of a fatty acid derivative target product.

The production of FAME is exemplary and was achieved by fermentation/culturing in the presence of methanol; other fatty acid ester derivatives can readily be produced by the strains described herein by addition of the appropriate alcohol to the culture media. For example, fatty acid ethyl esters can be produced by growing the strains in the presence of ethanol instead of methanol (see, e.g., Example 4 below), or fatty acid propyl esters can be produced by growing the strains in the presence of propanol, and so forth. This Example describes recombinant cells/microbes that produce fatty acid esters by engineering them to express an ester synthase. The production of fatty esters, however, is exemplary. As discussed above, other fatty acid derivatives can be produced by the strains described herein, by the expression of the appropriate fatty acid derivative enzymes.

### Example 4: Production of FAEE with Reduced Byproducts by Recombinant Strains Expressing a Heterologous R-Specific Enoyl-CoA Hydratase and a Heterologous Trans-2-Enoyl-CoA Reductase

This Example demonstrates, as described above and elsewhere herein, that other fatty acid esters, besides FAME, can be produced by the recombinant cells or microbes provided herein, by culturing in the presence of another alcohol besides methanol.

In particular, this Example demonstrates the production of a fatty acid ester composition, exemplified herein by a fatty acid ethyl ester (FAEE) composition, with a reduced amount of 3-hydroxy (3-OH) fatty acid ester byproducts, exemplified herein by 3-OH FAEE, by engineering bacterial strains (exemplified herein by E. coli) to heterologously express an enoyl-CoA hydratase and a trans-2-enoyl-CoA reductase.

An E. coli MG1655 derivative strain was engineered to lack (i.e., delete or knockout) acyl-CoA dehydrogenase (FadE; SEQ ID NO:26), to overexpress the transcriptional regulator FadR (from E. coli; SEQ ID NO:31), and to overexpress a heterologous acyl-CoA synthetase (FadD3 from P. putida; UniProtKB Accession No. Q88PT5; SEQ ID NO:32). As described elsewhere herein, FadE attenuation or deletion is optional. This base strain, denoted KTT.529, also contained a plasmid derived from a pCL1920-derivative vector, containing an p15A replicon, a kanamycin resistance marker, and a nucleic acid sequence/gene encoding an ester synthase from Limnobacter (UniProtKB Accession No. A6GSQ9; SEQ ID NO:29).

Two additional plasmids derived from a pCL1920-derivative vector, containing an SC101 replicon, and a spectinomycin resistance marker, were constructed. One contained nucleic acid sequences encoding only an acyl-ACP thioesterase from Cuphea hookeriana (FatB2; UniProtKB Accession No. Q39514) (SEQ ID NO: 33), and another also contained nucleic acid sequences encoding the R-specific enoyl-CoA hydratase PhaJ4 from P. putida (“PhaJ4_P.put”) and the trans-2-enoyl-CoA reductase TER (or FabV) from T. denticola (“TER_T.den”). The first plasmid (containing the thioesterase) was transformed into strain base strain KTT.529 to generate control strain sAZ.1148, and the two plasmids were transformed into base strain KTT.529 to generate strain KTT.552 (“PhaJ-Ter” strain).

The resulting strain KTT.552 (“PhaJ-Ter” strain), and the control strain sAZ.1148, were subjected to small scale fermentation in the presence of ethanol and product analysis as described in Example 1. As shown in Table 9, control strain sAZ.1148 produced fatty acid ethyl esters (FAEE) and 3-hydroxy fatty acid ethyl ester (3-OH FAEE) byproducts, whereas strain KTT.552, which heterologously expressed both an R-specific enoyl-CoA hydratase and a trans-2-enoyl-CoA reductase, produced almost exclusively fatty acid ethyl esters (FAEE) and only trace amounts (˜0.2%) of 3-hydroxy fatty acid ethyl ester (3-OH-FAEE) byproducts. The predominant chain length of the fatty acid derivatives produced by both strains was C8 (due to the specificity/selectivity of the FatB2 thioesterase), and the most abundant product was octanoic acid ethyl ester.

TABLE 9
FAEE Composition Produced by Recombinant strains
with and without Enoyl-CoA Hydratase and
Trans-2-Enoyl-CoA Reductase Expression.

| | | | | Total | |
|---|---|---|---|---|---|
| | | | Total | 3-OH | % 3-OH |
| | | Trans-2- | FAEE | FAEE | FAEE |
| | Enoyl-CoA | Enoyl-CoA | Titer* | Titer | (of Total |
| Strain | Hydratase | Reductase | (mg/L) | (mg/L) | FAS**) |
| AZ.1148 | none | none | 2780 | 1520 | 35.3 |
| (Control) | | | | | |
| KTT.552 | PhaJ4_P.put | TER_T.den | 6310 | 14 | 0.2 |

*Total FAEE Titer includes FAEE without a 3-OH group and 3-OH FAEE byproduct.
**FAS = fatty acid species

Thus, this example demonstrates that expression of an R-specific enoyl-CoA hydratase and a trans-2-enoyl-CoA reductase significantly reduces or eliminates (or substantially eliminates) 3-hydroxy fatty acid ethyl ester byproducts in recombinant strains (or cells, microbes, or microorganisms) that express an acyl-ACP dependent fatty acid biosynthetic pathway for the production of a fatty acid derivative target product (e.g., fatty acid ethyl ester (FAEE) target product). As discussed above, the production of fatty acid ethyl esters is exemplary, and the production of other types of fatty acid esters can be achieved by culturing (or growing or fermenting) the recombinant strains/cells/microbes/microorganisms in the presence of the appropriate alcohol (e.g., methanol for FAME, or propanol for fatty acid propyl esters, etc.). Additionally or alternatively, other fatty acid derivative products, such as, but not limited to, for example, free fatty acids, fatty alcohols, fatty alcohol acetate esters, fatty aldehydes, fatty amines, fatty amides, omega-hydroxy fatty acids, omega-hydroxy fatty acid esters, diacids, diols, and diesters, can be produced by recombinant cells/strains/microbes/microorganisms that express, or are engineered to express or overexpress, the appropriate fatty acid derivative biosynthetic pathways or enzymes.

### Example 5: Production of Fatty Alcohol Compositions with Reduced Byproducts by Recombinant Strains Expressing a Heterologous Enoyl-CoA Hydratase and a Heterologous Trans-2-Enoyl-CoA Reductase

As discussed above and elsewhere herein, the recombinant cells, strains, microbes, or microorganisms provided herein can be engineered to produce different fatty acid derivatives, such as, for example, free fatty acids, fatty esters, fatty alcohols, fatty aldehydes, fatty diols, fatty diacids, fatty diesters, etc., with reduced byproducts, namely, the 3-hydroxy versions of the fatty acid derivatives. Such recombinant cells, strains, microbes, or microorganisms express, or are engineered to express or overexpress, the appropriate fatty acid derivative biosynthetic pathways or fatty acid derivative enzymes that are required to produce a particular fatty acid derivative. Such pathways and enzymes are described elsewhere herein and/or are known in the art.

The following example illustrates the production of a fatty alcohol composition with a reduced amount (e.g., titer, yield, and/or percentage) of 1,3 fatty diol byproducts, by engineering bacterial strains (exemplified herein by E. coli) to express a heterologous enoyl-CoA hydratase and a heterologous trans-2-enoyl-CoA reductase.

To achieve the production of fatty alcohols, the recombinant cell or microorganism can express an acyl-CoA synthetase, which converts a fatty acid to a fatty acyl-CoA; an acyl-CoA reductase, which converts the fatty acyl-CoA to a fatty aldehyde; and an alcohol dehydrogenase, which converts the fatty aldehyde to a fatty alcohol. Alternatively, a fatty alcohol forming acyl-CoA reductase can be expressed to convert the acyl-CoA to a fatty alcohol. The recombinant cell or microorganism can further express an acyl-ACP thioesterase, which converts an acyl-ACP to a fatty acid. Any one or more, or all, of the acyl-ACP thioesterase, acyl-CoA synthetase, acyl-CoA reductase, a fatty alcohol forming acyl-CoA reductase, and/or alcohol dehydrogenase can be a native sequence (i.e., naturally occurring in the recombinant cell or microorganism) that is expressed or overexpressed, or can be a heterologous sequence (i.e., derived from a different species than the recombinant cell or microorganism) that is expressed or overexpressed, or a combination thereof.

For example, a bacterial strain, such as an E. coli MG1655 derivative strain, is engineered to overexpress an acyl-CoA synthetase (e.g., FadD), such as by replacing the native promoter with a synthetic or heterologous promoter, or to express or overexpress a heterologous acyl-CoA synthetase, such as FadD3 from P. putida (UniProtKB Accession No. Q88PT5; SEQ ID NO: 32). The strain can optionally be engineered to lack (i.e., delete or knockout) acyl-CoA dehydrogenase (FadE; SEQ ID NO:26). A heterologous acyl-CoA reductase from Acinetobacter (e.g., UniProtKB Accession No. Q6F7B8; SEQ ID NO:36) or a heterologous acyl-CoA reductase from Marinobacter (GenBank Accession No. ABM19582; SEQ ID NO:37), and an alcohol dehydrogenase from Acinetobacter baylyi (UniProtKB Accession No. Q6F6R9; SEQ ID NO: 34) are cloned into a pACYC-derivative vector comprising a p15A replicon and a kanamycin resistance marker (the “first plasmid”), under the control of an IPTG-inducible Ptrc promoter. The vector additionally can encode one or more of (i) a heterologous acyl-ACP thioesterase (e.g., FatB1 from Umbellularia californica, or FatB2 from Cuphea hookeriana); (ii) transcriptional regulator FadR; and (iii) a β-keto-acyl-ACP synthase I. This vector is transformed into the engineered bacterial strain, e.g., the engineered E. coli MG1655 derivative strain, to produce a control strain.

A second plasmid, which contains an SC101 replicon and a spectinomycin resistance marker, is prepared that comprises an operon comprising an enoyl-CoA hydratase gene, such as, for example, phaJ from P. putida or P. aeruginosa (see, e.g., Examples 2-4), or any one of the R-3-hydroxy-acyl-CoA dehydratases or R-specific enoyl-CoA hydratases listed in Table 1, and a trans-2-enoyl-CoA reductase gene, such as, for example, ter (or fabV) from T. denticola (“TER_T.den”) (see, e.g., Examples 3 and 4), or any one of the trans-2-enoyl-CoA reductases listed in Table 2, under the control of an IPTG-inducible PT5 promoter. The two plasmids are transformed into the E. coli MG1655 derivative strain described above.

The resulting strain (containing both plasmids), and a control strain that does not express the R-specific enoyl-CoA hydratase and the trans-2-enoyl-CoA reductase (i.e., that does not contain the second plasmid), are subjected to small scale fermentation and product analysis as described in Example 1.

The expression of an acyl-CoA synthetase, an acyl-CoA reductase, and an alcohol dehydrogenase (and an acyl-ACP thioesterase) in the control strain results in the production of a fatty alcohol composition which contains 3-hydroxy derivative byproducts, i.e., 1,3-fatty diols. The strain that also expresses the enoyl-CoA hydratase and the trans-2-enoyl-CoA reductase produces a fatty alcohol composition with a reduced or undetectable amount of 1,3-fatty diol byproducts. The biosynthetic fatty alcohol pathways and enzymes described in this Example are exemplary; the same fatty alcohol composition can be produced by utilizing the same enzymes/pathway described herein from different organisms/species, or by utilizing a different biosynthetic pathway or different enzymes. For example, a fatty alcohol composition also can be produced by expressing or overexpressing one or more of an acyl-CoA thioesterase, a carboxylic acid reductase, and an alcohol dehydrogenase, or by expressing or overexpressing a fatty alcohol forming acyl-CoA reductase.

This example demonstrates that expression of an R-specific enoyl-CoA hydratase and a trans-2-enoyl-CoA reductase reduces the amount (e.g., titer, yield, percentage) of, or eliminates or substantially eliminates, 1,3 fatty diol byproducts present in a composition of fatty alcohols produced by a recombinant strain that expresses an acyl-ACP dependent fatty acid biosynthetic pathway.

### Example 6: Production of Fatty Alcohol Acetate Compositions with Reduced Byproducts by Recombinant Strains Expressing a Heterologous Enoyl-CoA Hydratase and a Heterologous Trans-2-Enoyl-CoA Reductase

The following example illustrates the production of a fatty alcohol acetate composition with a reduced amount (e.g., titer, yield, and/or percentage) of 1,3 fatty alcohol diacetate byproducts, by engineering bacterial strains (exemplified herein by E. coli strains) to express a heterologous R-specific enoyl-CoA hydratase and a heterologous trans-2-enoyl-CoA reductase.

To achieve the production of fatty alcohol acetates, the recombinant cell or microorganism can express an acyl-CoA synthetase, which converts a fatty acid to a fatty acyl-CoA; an acyl-CoA reductase, which converts the fatty acyl-CoA to a fatty aldehyde; an alcohol dehydrogenase, which converts the fatty aldehyde to a fatty alcohol; and an alcohol-O-acetyl-transferase, which converts the fatty alcohol to a fatty alcohol acetate. The recombinant cell or microorganism can further express an acyl-ACP thioesterase, which converts an acyl-ACP to a fatty acid. Alternatively, the acyl-CoA can be converted to a fatty alcohol by expressing a fatty alcohol forming acyl-CoA reductase. Any one or more, or all, of the acyl-ACP thioesterase, acyl-CoA synthetase, acyl-CoA reductase, alcohol dehydrogenase, fatty alcohol forming acyl-CoA reductase, and/or alcohol-O-acetyl-transferase can be a native sequence (i.e., naturally occurring in the recombinant cell or microorganism) that is expressed or overexpressed, or can be a heterologous sequence (i.e., derived from a different species than the recombinant cell or microorganism) that is expressed or overexpressed, or a combination thereof.

For example, a bacterial strain, such as an E. coli MG1655 derivative strain, is engineered to overexpress an acyl-CoA synthetase (e.g., FadD), such as by replacing the native promoter with a synthetic or heterologous promoter, or is engineered to express or overexpress a heterologous acyl-CoA synthetase, such as FadD3 from P. putida (UniProtKB Accession No. Q88PT5; SEQ ID NO:32). The strain can optionally be engineered to lack (i.e., delete or knockout) acyl-CoA dehydrogenase (FadE; SEQ ID NO:26). A heterologous acyl-CoA reductase from Acinetobacter baylyi (UniProtKB Accession No. Q6F7B8; SEQ ID NO:36) or a heterologous acyl-CoA reductase from Marinobacter aquaeolei (GenBank Accession No. ABM19582; SEQ ID NO:37), an alcohol dehydrogenase from Acinetobacter baylyi (UniProtKB Accession No. Q6F6R9; SEQ ID NO:34), and an alcohol-O-acetyl-transferase from Saccharomyces (UniProtKB Accession No. Q6XBT3; SEQ ID NO:35), are cloned into pACYC-derivative vector comprising a p15A replicon and a kanamycin resistance marker (the “first plasmid”), under the control of an IPTG-inducible Ptrc promoter. The vector additionally can encode one or more of (i) a heterologous acyl-ACP thioesterase (e.g., FatB1 from Umbellularia californica, or FatB2 from Cuphea hookeriana); (ii) transcriptional regulator FadR; and (iii) a β-keto-acyl-ACP synthase I. This vector is transformed into the engineered E. coli strain to produce a control strain.

A second plasmid, which contains an SC101 replicon and a spectinomycin resistance marker, is prepared that comprises an operon comprising an enoyl-CoA hydratase gene, such as, for example, phaJ from P. putida or from P. aeruginosa (see, e.g., Examples 2-4), or any one of the R-3-hydroxy-acyl-CoA dehydratases or R-specific enoyl-CoA hydratases listed in Table 1, and a trans-2-enoyl-CoA reductase gene, such as, for example, ter (fabV) from T. denticola (“TER_T.den”) (see, e.g., Examples 3 and 4), or any one of the trans-2-enoyl-CoA reductases listed in Table 2, placed under the control of an IPTG-inducible PT5 promoter. The two plasmids are transformed into the E. coli MG1655 derivative strain described above. The genes, encoded proteins/enzymes, and/or biosynthetic fatty alcohol pathways and enzymes described herein are exemplary; the native genes and enzymes for a particular recombinant host cell or microbe also can be used, for example, by overexpression in the native host. Alternatively or additionally, heterologous genes/enzymes from other species (e.g., homologs or enzymes with the same activities), and/or a different biosynthetic pathway or different enzymes, can be expressed in the recombinant host cell or microbe or strain. For example, a fatty alcohol composition also can be produced by expressing or overexpressing one or more of an acyl-CoA thioesterase, a carboxylic acid reductase, and an alcohol dehydrogenase, or by expressing or overexpressing a fatty alcohol forming acyl-CoA reductase, and the fatty alcohols can be converted to fatty alcohol acetates by expressing or overexpressing an alcohol-O-acetyl-transferase.

The resulting strain (containing both plasmids) and the control strain that does not express the R-specific enoyl-CoA hydratase and the trans-2-enoyl-CoA reductase (i.e., that does not contain the second plasmid), are subjected to small scale fermentation and product analysis as described in Example 1.

The expression of fatty alcohol biosynthetic enzymes (e.g., an acyl-ACP thioesterase, an acyl-CoA synthetase, an acyl-CoA reductase, and an alcohol dehydrogenase), and an alcohol-O-acetyl-transferase, in the control strain, results in the production of a fatty alcohol acetate composition which contains 3-hydroxy derivative byproducts, i.e., 1,3 fatty alcohol diacetate byproducts. The strain that also expresses the enoyl-CoA hydratase and the trans-2-enoyl-CoA reductase produces a fatty alcohol acetate composition with a reduced (or undetectable) amount of 1,3 fatty alcohol diacetate byproducts.

This example demonstrates that expression of an R-specific enoyl-CoA hydratase and a trans-2-enoyl-CoA reductase reduces the amount (e.g., titer, yield, and/or percentage) of, or eliminates or substantially eliminates, 1,3 fatty alcohol diacetate byproducts present in a composition of fatty alcohol acetates produced in a recombinant strain with an acyl-ACP dependent fatty acid biosynthetic pathway.

### Example 7: Production of Fatty Acid Derivatives with 3-Oxo (3-Keto) Byproducts by Recombinant Strains and Reduction of the 3-Oxo Byproducts by Expression of a Heterologous 3-Keto-Acyl-CoA Reductase, a Heterologous Enoyl-CoA Hydratase, and a Heterologous Trans-2-Enoyl-CoA Reductase

As discussed above, recombinant cells or microbes comprising an acyl-ACP dependent fatty acid biosynthetic pathway can result in the production of 3-oxo byproducts (from 3-keto-acyl-ACP intermediates), in addition to the production of 3-hydroxy byproducts (from 3-hydroxy-acyl-ACP intermediates). The expression of an enoyl-CoA hydratase (or a 3-hydroxy-acyl-CoA dehydratase) and optionally, a trans-2-enoyl-CoA reductase, cannot reduce or eliminate the production of 3-oxo byproducts, because the enoyl-CoA hydratase (or 3-hydroxy-acyl-CoA dehydratase) cannot convert 3-oxo acyl-CoA to trans-2-enoyl-CoA. Instead, the expression of an additional enzyme, such as a β-keto-(3-keto)-acyl-CoA reductase (or a 3-oxoacyl-CoA reductase, or a 3-hydroxy-acyl-CoA dehydrogenase), is necessary to convert the 3-oxo-acyl-CoAs to 3-hydroxy-acyl-CoAs, which can then be converted by the enoyl-CoA hydratase (or 3-hydroxy-acyl-CoA dehydratase) to trans-2-enoyl-CoAs. The rest of the byproduct reduction or bypass pathway, towards acyl-CoAs is the same, since both the pathways to reduce 3-hydroxy byproducts and 3-oxo byproducts go through a 3-hydroxy-acyl-CoA intermediate.

The following example illustrates the production of 3-oxo (or 3-keto) byproducts, such as 3-oxo-octanoic acid ethyl ester (3-keto-octanoic acid ethyl ester) and 3-oxo-decanoic acid ethyl ester (3-keto-decanoic acid ethyl ester), by strains expressing a heterologous enoyl-CoA hydratase and a heterologous trans-2-enoyl-CoA reductase, as well as an acyl-ACP dependent biosynthetic pathway and the fatty acid derivative enzymes necessary for production of fatty acid esters. Also illustrated in this Example is the the reduction (or elimination or substantial elimination) of the 3-oxo byproducts, by engineering the strains to further express a heterologous 3-keto-acyl-CoA reductase.

An E. coli MG1655 derivative strain was engineered to encode an attenuated acyl-CoA dehydrogenase (FadE; SEQ ID NO:26); a heterologous acyl-CoA synthetase (FadD3 from P. putida; UniProtKB Accession No. Q88PT5; SEQ ID NO:32); a heterologous enoyl-CoA hydratase PhaJ4 from P. putida (“PhaJ4_P.put”); and a trans-2-enoyl-CoA reductase (TER or FabV) from T. denticola (“TER_T.den”); and to overexpress the transcriptional regulator FadR. As described elsewhere herein, FadE attenuation or deletion is optional.

A plasmid comprising an SC101 replicon and a spectinomycin resistance marker was prepared that contained: a first operon comprising a gene encoding an ester synthase from Limnobacter (UniProt_A6GSQ9; SEQ ID NO:29) under the control of an IPTG-inducible PT5 promoter; and a second operon containing a gene encoding an acyl-ACP thioesterase from Cuphea hookeriana (FatB2, UniProt_Q39514; SEQ ID NO:33), under the IPTG-inducible Ptrc promoter. This plasmid was then transformed into the engineered E. coli MG1655 derivative strain described above, to generate strain sven.1169.

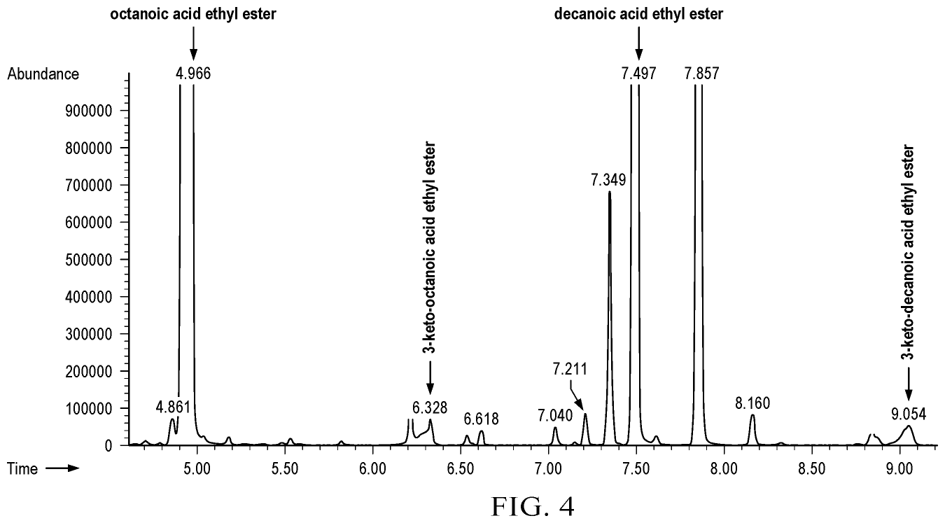

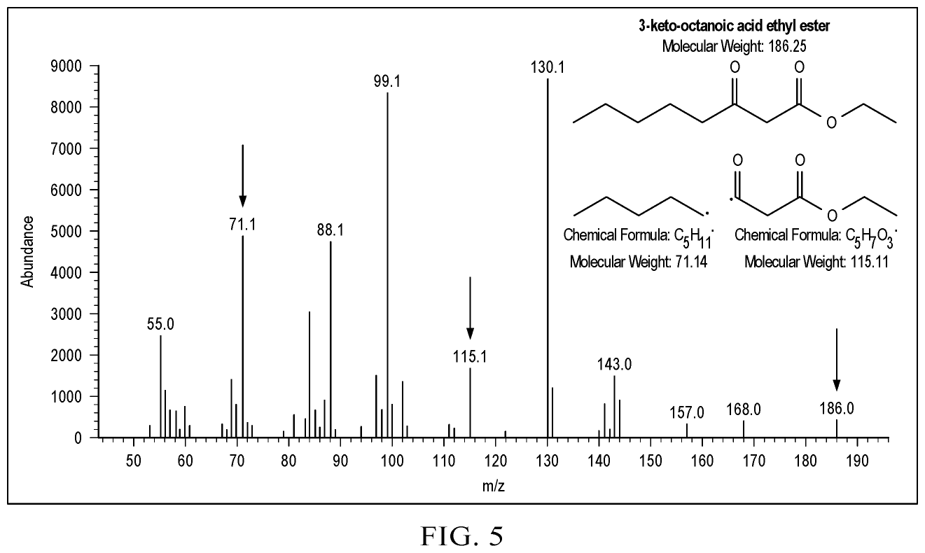

The resulting strain sven. 1169 was subjected to small scale fermentation in the presence of ethanol and to product analysis as described in Example 1. Strain sven.1169 produced mainly fatty acid ethyl esters (FAEE), particularly, octanoic acid ethyl ester (2 g/L titer) and decanoic acid ethyl ester (0.6 g/L titer), and produced small amounts of 3-hydroxy fatty acid ethyl esters (3-OH FAEE). However, the strain also produced a total of about 2% of 3-keto-octanoic acid ethyl ester and 3-keto-decanoic acid ethyl ester byproducts. FIG. 4 shows a GC chromatograph of fatty acid ethyl esters produced by strain sven.1169. The major peaks with retention times of approximately 4.966 minutes and 7.497 minutes were identified as octanoic acid ethyl ester and decanoic acid ethyl ester, respectively, while the minor peaks at retention times of approximately 6.328 minutes and 9.054 minutes were identified as 3-keto-octanoic acid ethyl ester (3-oxo-octanoic acid ethyl ester) and 3-keto-decanoic acid ethyl ester (3-oxo-decanoic acid ethyl ester), respectively, based on their unique ion fragmentation patterns by LC-MS. For example, FIG. 5 shows the characteristic ion fragments of 3-keto-octanoic acid ethyl ester, which has a molecular weight of 186.25 g/mol, and produces characteristic ion fragments with molecular weights of approximately 71.1 and 115.1 g/mol.

In order to reduce or eliminate (or substantially eliminate) the production of the 3-oxo byproducts by strain sven.1169, another plasmid, comprising a p15A replicon and a kanamycin resistance marker, and encoding a heterologous β-keto-acyl-CoA reductase, for example one from Cupriavidus necator (see, e.g., UniProtKB Accession No. P14697; SEQ ID NO: 38) or from Cupriavidus taiwanensis (see, e.g., UniProtKB Accession No. B3R6T4; SEQ ID NO: 41), under the control of an IPTG-inducible Ptrc promoter, is prepared and transformed into strain sven.1169. The B-keto-acyl-CoA reductases described herein as exemplary; any polypeptide or enzyme with activity to convert 3-oxo-acyl-CoAs to the corresponding 3-hydroxy-acyl-CoAs can be used (i.e., with activity belonging to EC 1.1.1.35, EC 1.1.1.36, or EC 1.1.1.157) can be used, such as, but not limited to, the enzymes listed in Table 2A or in any one of SEQ ID NOs: 38-45.

The resulting strain is subjected to small scale fermentation in the presence of ethanol and product analysis as described in Example 1. The strain produces mainly fatty acid ethyl esters (FAEE), e.g., octanoic acid ethyl ester and decanoic acid ethyl ester, and in contrast to strain sven.1169, the strain expressing the heterologous B-keto-acyl-CoA reductase does not produce or produces only insignificant amounts of 3-oxo byproducts, such as 3-oxo FAEE byproducts, e.g., 3-oxo-octanoic acid ethyl ester and 3-keto-decanoic acid ethyl ester byproducts. The strain also does not produce, or produces only insignificant amounts of 3-hydroxy byproducts, such as 3-hydroxy fatty acid ethyl esters.

Thus, this example demonstrates that expression of a β-keto-acyl-CoA reductase, an R-specific enoyl-CoA hydratase, and a trans-2-enoyl-CoA reductase significantly reduces or eliminates (or substantially eliminates) the production of 3-hydroxy fatty acid derivative byproducts (exemplified herein by 3-hydroxy FAEEs), and significantly reduces or eliminates (or substantially eliminates) the production of 3-oxo fatty acid derivative byproducts (exemplified herein by 3-oxo FAEEs), in recombinant strains that expresses an acyl-ACP dependent fatty acid biosynthetic pathway for the production of a fatty acid derivative target product (e.g., fatty acid ethyl ester (FAEE) target product).

All references, including publications, patent applications, and patents, cited herein, are hereby incorporated by reference to the same extent as if each reference were individually and specifically indicated to be incorporated by reference and were set forth in its entirety herein.

Particular embodiments of this invention are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Sequence Table

| SEQ ID | | |
|---|---|---|
| NO: | Description | Sequence |
| 1 | MaoC family | MSQVTNTPYEALEVGQKAEYKKSVEERDIQLFAAMSGDHNPVHLDAEFAAKSM |
| | dehydratase | FRERIAHGMFSGALISAAVACTLPGPGTIYLGQQMSFQKPVKIGDTLTVRLEILEK |
| | [PhaJ1_P.put] | LPKFKVRIATNVYNQNDELVVAGEAEILAPRKQQTVELVSPPNFVAS |
| 2 | MaoC family | MPHVPVTELSQYVGKELGHSEWLKIDQQRINLFAEATGDFQFIHVDPEKAAKTP |
| | dehydratase | FGGTIAHGFLTLSLIPKLIEDILVLPQGLKMVVNYGLDSVRFIQPVKVDSRVRLKV |
| | [PhaJ4_P.put] | KLGEVVEKKPGQWLLKAIATLEIEGEEKPAYIAESLSLCFV |
| 3 | MaoC family | MPTAWLDLPAPPALPGLFLRAALRRGIRGKALPERGLRSQVTVDPKHLERYRQV |
| | dehydratase | CGFRDDGLLPPTYPHILAFPLQMALLTDKRFPFPLLGLVHLENRIDVLRALGGLG |
| | [PhaJ3_P.aer | PFTVSVAVENLQPHDKGATFSIVTRLEDQLGLLWVGDSKVLCRGVKVPGEIPPK |
| | | AEQEPLPLEPVDNWKAPADIGRRYARAAGDYNPIHLSAPSAKLFGFPRAIAHGL |
| | | WNKARSLAALGERLPASGYRVEVRFQKPVLLPASLTLLASAAAADGQFSLRGK |
| | | DDLPHMAGHWSRLQG |
| 4 | MaoC family | MPFVPVAALKDYVGKDLGHSEWLTIDQERVDQFAECTGDHQFIHVDPEKAAKT |
| | dehydratase | PFGGTIAHGFLSLSLIPKLMEGLLVLPEGLKMAVNYGLDTVRFIQPVRVGSRVRL |
| | [PhaJ4_P.aer] | GLTLLDVNEKNPGQWLIKARATLEIEGQEKPAYIAETLSLCFV |
| 5 | MaoC family | MPRVFEHPEQLLACVGETLGESEWLSVEQSRIDRFAEATGDHQWIHVDPVRAAS |
| | dehydratase | GPYGACIAHGYLTLALVNLFLPQIVEVRGIAMGVNYGCDRVRFPNVVKAGARV |
| | [Pseudomonas | RGRAELVAAEAVKGAVQATIRVSVEIDGEDRPGCVVDTISRYYPA |
| | citronellolis] | |
| 6 | MaoC family | MPSVPVAELKDYVGKELGKSEWLTIDQQRINQFAECTGDHQFIHVDPEKAKLTP |
| | dehydratase | FGTTIAHGFLSLSLVPMLMEKIMIMPQGLKMAVNYGLDSVRFIQPVKVNSKVRL |
| | [Pseudomonas | VVTLTDANEKNPGQWLLKARAVLEIEGQEKPAYIAEPLTLCFV |
| | mendocina] | |
| 7 | MaoC9 domain | MSRQWHDLHNPAARASLYLRAASKRSISGERLPDDGLRCFIRVQPGNLGAYRRL |
| | protein | CHFPDDGRLPGTYPHVMAFALQLQLMTADDFPFPLLGLVHLHNRIEVLRPLGAV |
| | [Pseudomonas | EGLRFAVYTHNLQRHAKGGTFDLVTEAEDGIGLLWRETSRMLVRGLALEGEAS |
| | putida KT2440] | EPADPEPDPLAEATRWYADSDIGRRYAKVCGDYNPIHLSAVSARLFGFPTAIAHG |
| | | MWTKAMALAALRGHLPHSGYAFEVDFRKPVRLPSEVVLSASEAGPSGQLRLDG |
| | | HGDVLHMVGRWESL |
| 8 | MaoC dehydratase | MRPGLDGGLSMSRHWHDLHSPASRTSLYLRAASKRSISGDQLPDDGLRCFIRVQ |
| | [Pseudomonas | PGNLAAYRRLCHFTDDGRLPGTYPHVMAFTLQLQLMTAPNFPFPLLGLVHLHN |
| | plecoglossicida] | RIEVLRPLGGIEGLRFAVHAGNLQAHAKGGTFDLVTEAEDGIGLLWRETSRMLV |
| | | RGLKLESQAGEPAEDEPQALPEATRWYADSDIGRRYAKVCGDYNPIHLSAASAR |
| | | LFGFPTAIAHGMWSKAMALAALRGHLPHSGFAFEVDFRKPVRLPSEVVLGASEA |
| | | GTTGELRLDGHGGVLHMVGRWDRL |
| 9 | MaoC family | MPHVPVTELSQYVGRELGRSAWLKIDQQRINLFAEATGDFQFIHVDPEKAAKTP |
| | dehydratase | FGSTIAHGFLTLSLIPKLIEDILVLPEGLKMVVNYGLDSVRFIQPVKVDSQVRLKV |
| | [Pseudomonas | DLAEVTEKRPGQWLLKAIATLEIDGEEKPAFIAESLSLCFV |
| | plecoglossicida] | |
| 10 | (R)-specific enoyl- | MPYVPVTELSQYVGKELGRSQWLKIDQERINLFAEATGDFQFIHVDPVKAAKTP |
| | CoA hydratase | FGSTIAHGFLTLSLIPKLMEDILVLPEGLKMVVNYGLDSVRFIQPVKVDSQVRLK |
| | [Pseudomonas | VDLTDATEKKPGQWLLKATVTLEIEGEEKPAYIAEPLSLCFV |
| | mosselii] | |
| 11 | MaoC family | MPLVPVTELSQYVGKELGCSDWLKIDQERINLFAEATGDFQFIHVDPVKAAKTP |
| | dehydratase | FGCTIAHGFLTLSLISKLIEDVVVLPEGNKMVVNYGLDSVRFIQPVKVDSKVRLK |
| | [Pseudomonas | VELTEATEKKPGQWLLKSKVALEIEGEEKPAYIAEPLILCFV |
| | plecoglossicida] | |
| 12 | MaoC family | MPLVPVTALTQYVGKSLGHSAWLTIDQQRIDRFAEATGDHQFIHVDPQRAAQTA |
| | dehydratase | FGGTIAHGFLTLSLIPTLIEDILVLPQGLKMVVNYGLDSVRFIQPVKVDSQVRLHV |
| | [Pseudomonas | ELADVQQKRPGQWLLKAIATLEIKDEDKPAYRAESLSLCFV |
| | fulva] | |
| 13 | MaoC family | MPLVPVTALTQYIGKSLGHSAWLTIDQQRIDRFAEATGDHQFIHVDPQRAAQTV |
| | dehydratase | FGCTIAHGFLTLSLIPALIEDILVLPQGLKMVVNYGLGSVRFIQPVKVDSQVRLHV |
| | [Pseudomonas | ELADVQQKRPGQWLLKAIATLEIKDEDKPAYRAESLSLCFV |
| | fulva] | |
| 14 | trans-2-enoyl-CoA | MIVKPMVRNNICLNAHPQGCKKGVEDQIEYTKKRITAEVKAGAKAPKNVLVLG |
| | reductase family | CSNGYGLASRITAAFGYGAATIGVSFEKAGSETKYGTPGWYNNLAFDEAAKRE |
| | protein | GLYSVTIDGDAFSDEIKAQVIEEAKKKGIKFDLIVYSLASPVRTDPDTGIMHKSVL |
| | [Treponema | KPFGKTFTGKTVDPFTGELKEISAEPANDEEAAATVKVMGGEDWERWIKQLSKE |
| | denticola] | GLLEEGCITLAYSYIGPEATQALYRKGTIGKAKEHLEATAHRLNKENPSIRAFVS |
| | | VNKGLVTRASAVIPVIPLYLASLFKVMKEKGNHEGCIEQITRLYAERLYRKDGTI |
| | | PVDEENRIRIDDWELEEDVQKAVSALMEKVTGENAESLTDLAGYRHDFLASNGF |
| | | DVEGINYEAEVERFDRI |
| 15 | Trans-2-enoyl-CoA | MSCPASPSAAVVSAGALCLCVATVLLATGSNPTALSTASTRSPTSLVRGVDRGL |
| | reductase [Euglena | MRPTTAAALTTMREVPQMAEGFSGEATSAWAAAGPQWAAPLVAAASSALALW |
| | gracilis] | WWAARRSVRRPLAALAELPTAVTHLAPPMAMFTTTAKVIQPKIRGFICTTTHPIG |
| | | CEKRVQEEIAYARAHPPTSPGPKRVLVIGCSTGYGLSTRITAAFGYQAATLGVFL |
| | | AGPPTKGRPAAAGWYNTVAFEKAALEAGLYARSLNGDAFDSTTKARTVEAIKR |
| | | DLGTVDLVVYSIAAPKRTDPATGVLHKACLKPIGATYTNRTVNTDKAEVTDVSI |
| | | EPASPEEIADTVKVMGGEDWELWIQALSEAGVLAEGAKTVAYSYIGPEMTWPV |
| | | YWSGTIGEAKKDVEKAAKRITQQYGCPAYPVVAKALVTQASSAIPVVPLYICLL |
| | | YRVMKEKGTHEGCIEQMVRLLTTKLYPENGAPIVDEAGRVRVDDWEMAEDVQ |
| | | QAVKDLWS |
| | | QVSTANLKDISDFAGYQTEFLRLFGFGIDGVDYDQPVDVEADLPSAAQQ |
| 16 | trans-2-enoyl-CoA | MIIKPVIQGVVARSAHPLGCQQAVLNQIRYVQAAPQISGGPKKVLVLGASSGFGL |
| | reductase family | ASRISLAFGGAKADTIGISFEKGPSEKGAGTAGWYNNVFFRQEAEKHGLIAKNFV |
| | protein | GDAFSSEMRKQVIDYIKKEFGGHVDLVVYSLATGIRPNPETGELWRSSIKTLDKP |
| | [Enterovibrio | LSGPTIDLERDVMLPLDVGVATEDELDATVKVMGGEDWESWIAVLSDAGVLAK |
| | coralii] | GCKTLAYSYIGPEFTYPIYHRGTLGKAKDHLHKTADVLNQTLQDLEGEAYVAV |
| | | CKALVTKASVFIPAFSPYILSLFKVMKENGTHEGCIEQMQRLFTERLYRNGDHVP |
| | | VDNARLIRMDDWELAPETQSAVAALMAEMTADNFQHVGDYQGYKQDFLQLN |
| | | GFDFAGIDYDMPVDMAFLMSLRP |
| 17 | trans-2-enoyl-CoA | MKIDKKFKGNVARSVNPYGCHQEVLNQIKYVKENGHYDGAKKALIIGGSSSYG |
| | reductase family | LASRITQAFGSGADTISVAFERPIKDETMLGTAGWWNNIYFKQEAEKAGLIAKNF |
| | protein | NGDCFTEKMKEQVSEYIKDEFGGKIDLLVYSVAAPKRANPENTDEVWRSQMKPI |
| | [Lactobacillus | GKDVTGYNVNLEQDALVEQTIEAATPEEVADTVHVMGGEDWELWVKFLKDQG |
| | oligofermentans] | LLADGFKTILYSYIGSPVTYDFYHEGTLGKAKDAAEESSHRIGKIIEDINGESLISV |
| | | SKAVTTKASSVIPIFPVYCIALYKVMQEKGTHETPIMHKDRLFRDMVYGNKREID |
| | | DHGRLRPDSWELDEDTQRKTLDLMNKITPENFNTDLTGYETFKKEFLQINGFEV |
| | | EGSNNETVDFDEISKLEP |
| 18 | trans-2-enoyl-CoA | MLGKMIKIDKKFKGNVARSVNPYGCHQEVLNQIKYVKENGHYDGAKKALIIGG |
| | reductase family | SSSYGLASRITQAFGSGADTISVAFERPIKDETMLGTAGWWNNIYFKQEAEKAGL |
| | protein | IAKNFNGDCFTEKMKEQVSEYIKDEFGGKIDLLVYSVAAPKRANPENTDEVWRS |
| | [Paucilactobacillus | QMKPIGKDVTGYNVNLEQDALVEQTIEAATPEEVADTVHVMGGEDWELWVKF |
| | oligofermentans] | LKDQGLLADGFKTILYSYIGSPVTYDFYHEGTLGKAKDAAEESSHRIGKIIEDING |
| | | ESLISVSKAVTTKASSVIPIFPVYCIALYKVMQEKGTHETPIMHKDRLFRDMVYG |
| | | NKREIDDHGRLRPDSWELDEDTQRKTLDLMNKITPENFNTDLTGYETFKKEFLQI |
| | | NGFEVEGSNNETVDFDEISKLEP |
| 19 | trans-2-enoyl-CoA | MIIKPRIRGFICTTTHPVGCEANVKEQIAYTKAQGPIKNAPKRVLVVGASSGYGLS |
| | reductase family | SRIAAAFGGGASTIGVFFEKEGTEKKPGTAGFYNSVAFEKLAREEGLYAKSLNG |
| | protein [Vibrio | DAFSNEAKQKTIDLIKEDLGQVDMVVYSLASPVRKMPETGELIRSALKPIGETYT |
| | harveyi] | STAVDTNKDVIIEASVEPATEQEIQDTVTVMGGEDWELWINALSEAGVLADGCK |
| | | TVAYSYIGTELTWPIYWDGALGKAKMDLDRAAAALNEKLSTTGGSANVAVLKS |
| | | VVTQASSAIPVMPLYIAMVFKKMREEGVHEGCMEQIFRMFSQRLYKEDGSAAE |
| | | VDEVNRLRLDDWELREDIQQHCRDLWPQITSENLKELTDYVEYKEEFLKLFGFG |
| | | VDGVDYEADVNPAVETDFTQI |
| 20 | trans-2-enoyl-CoA | MIIKPMVRGNICLNAHPAGCKKSVEDQIAYTKKGGKRTSALKNALILGCSNGYG |
| | reductase family | LASRITASFGYGAATIGVSFEKAGSETKWGTPGWYNNLAFDQAAKEAGLTAVTI |
| | protein | DGDAFSDGIKETVIGEAKNRGMKFDLVVYSLASPVRVDPDTGVMHKSVLKPFG |
| | [Treponema | KAFCGKTLDPFTGKLSEITAEPATDEEAAATVKVMGGEDWQRWMDKLYKAGV |
| | lecithinolyticum] | LADGCITVAYSYIGPDATQALYRKGTIGKAKEHLEATAHTLNKEMAAFGGKAF |
| | | VSVNKGLVTRASAVIPVIPLYLASLFKVMKQKGNHEGCIEQMNRLFDERLYRSD |
| | | KQIPVDSENRIRIDDWELSDDVQSAVRALMEQVTDENSEKLTDLEGYRHDFLAA |
| | | NGFDIAGVDYQADIERFDRL |
| 21 | trans-2-enoyl-CoA | MIIKPMVRGNVCLNSHPKGCKKAVEHQIAYTKKRSADFSSGTVKPKNALILGCS |
| | reductase family | NGYGLASRITAAFGYGAATIGVSFEKAGSETKWGTPGWYNNLAFDGAAKAEGL |
| | protein | VSITIEGDAFSDAVKAEVIKKAKEHGMNFDLVIYSLASPLRIDPDTGVTYKSVLK |
| | [Treponema pedis] | PFGKAFTGKTLDPFTGKLSEITAEPATEEEAAATVKVMGGEDWQRWIEQLSEAG |
| | | VLAEGCITAAYSYIGPEATQALYRKGTIGKAKEHLEKTAHILNDKMKTFNGKAF |
| | | VSVNKGLVTRASAVIPVIPLYLASLFKVMKEKGNHEGCIEQINRLFSERLYTKDG |
| | | KIPVDGENRIRIDDWELDDEVQAAVDKIMEKVTDDNSETLTDLAGYRHDFLAA |
| | | NGFDIDGIDYDCDVERCDGI |
| 22 | trans-2-enoyl-CoA | MSMKPMLRSNICLNAHPQGCKKAVEDQIAYTKKRAKSHPAGTAVPKNVLIVGC |
| | reductase family | SGGYGLASRITAAFGYGATTIGVSYEKAGSEKKWGTPGWYNNLAVDTAAKEAG |
| | protein | LVSVTIDGDAFSDAIKTQVIDEAKKLGIKFDLIIYSVASSVRTDPETGVTYRSALK |
| | [Treponema | PFGKPFTGKTLDPFTGALTEITAEPATDEEAFATVKVMGGEDWQRWIEKLGAAN |
| | vincentii] | LLAQGCITVAYSYIEPEATQALYRKGTIGKAKEHLEATAHALNTKLAALKGQAF |
| | | VSVNKGLVTRASAVIPVIPLYLASLFKVMKEKGTHEGCIEQINRLFDSRLYTTDG |
| | | VIPTDSEHRIRIDDWELDESVQSAVAEIMATVTDETSRERTDVDEYRHDFLAING |
| | | FDIAGIDYDAEIDRFDRI |
| 23 | trans-2-enoyl-CoA | MVIKPMIRSNICLNAHPAGCKKAVEDQIEYVKAQAAKRAETGKSVLKSSKYAPK |
| | reductase family | NVLVLGCSNGYGLASRITAAFGYGASTIGVSFEKAGSETKWGTPGWYNNLAFD |
| | protein | AAAKKENIKSITIDGDAFSHAVKQQVVDQAKAQNMKFDLVVYSLASPVRVDPD |
| | [Treponema sp.] | TNVMYKSVLKPIGKVFSGMTIDPFTGELKEISAEPANEEEIINTQKVMGGEDWAL |
| | | WIEKLEKENVLAENCITLAYSYVGPEVSHAIYRSGTIGKAKEHLEKTAQELTKK |
| | | MASYGGRAYVSVNKGLVTRASAVIPIIPLYLSVLFKVMKAKGTHEGCIEQIDRLF |
| | | AERLYISEDRTKTPIPVDEENRIRIDDLEMDSEVQAEVSRIMPTVTSENSAELTDL |
| | | EGYRHDFLATSGFDIDGVDYEADIEAFDRI |
| 24 | trans-2-enoyl-CoA | MIIKPMIRSNICINAHPVGCAKETERQIAYVQSRKANRGTKTAAEGGKAPKAVLV |
| | reductase family | LGCSTGYGLASRITAAFEYGAATIGVSFEKEGSEKKGGTPGWYNNLAFDRASKK |
| | protein | AGIPSVTLNMDAFSDDCRKAVVAEARKMGVAFDLVVYSLASPVRTDPETGVLY |
| | [Treponema | KSVLKPFGKPFSGQTVDMMSGELSVMSAEPANDDEAAQTVKVMGGEDWERWI |
| | brennaborense] | DQLAEAGVLAKGCKTVAYSYIGPALSHAIYRDGTIGGAKKHLEKTARALDSKLS |
| | | QKLGGEAFVSVNKGLVTRSSAVIPIIPLYLSVLFKVMKEKGTHEGCIEQMERLFA |
| | | ERLYTDGSVPVDAEHLIRMDDWELDPAVQAEVDKRLAQVTQDTLASLGDMDG |
| | | YRHDFLATNGFDVAGVDYDADVERMDVV |
| 25 | trans-2-enoyl-CoA | MVIKPMVRSNICINAHPVGCAKETENQIEYVKAQKKKRGIKNVKEGGKGPKTVL |
| | reductase family | VLGCSTGYGLASRIVSAFAYGADTIGVSYEKEATETKGGTPGWYNNTAFDAAA |
| | protein | KKAGLVSITINADAYADETRSQVIGEAKKLGRKFDLIIYSLASPVRTDPDTKVLY |
| | [Treponema | KSVIKPRGKPYSGKYIDIMSQTLKESSEEPATEEEIANTVKVMGGDDWRRWIKQ |
| | parvum] | LSEADVLAKGCRTVAYSYIGPELSHAIYRDGTIGTAKLDLEKAALDLNEELKSSV |
| | | GGGAFISVNKGLVTRSSAVIPIISLYLSILFKVMKEKGTHEGCIEQIERLFAERLYT |
| | | GADNSASDVPTDSEHRIRIDDWELADDVQKRCRERMAAVTQENLSQLCDLEGY |
| | | KHDFLATNGFDIAGVDYDKDVARMDSIS |
| 26 | acyl-CoA | MMILSILATVVLLGALFYHRVSLFISSLILLAWTAALGVAGLWSAWVLVPLAIIL |
| | dehydrogenase | VPFNFAPMRKSMISAPVFRGFRKVMPPMSRTEKEAIDAGTTWWEGDLFQGKPD |
| | (FadE) from E. coli | WKKLHNYPQPRLTAEEQAFLDGPVEEACRMANDFQITHELADLPPELWAYLKE |
| | | HRFFAMIIKKEYGGLEFSAYAQSRVLQKLSGVSGILAITVGVPNSLGPGELLQHY |
| | | GTDEQKDHYLPRLARGQEIPCFALTSPEAGSDAGAIPDTGIVCMGEWQGQQVLG |
| | | MRLTWNKRYITLAPIATVLGLAFKLSDPEKLLGGAEDLGITCALIPTTTPGVEIGR |
| | | RHFPLNVPFQNGPTRGKDVFVPIDYIIGGPKMAGQGWRMLVECLSVGRGITLPS |
| | | NSTGGVKSVALATGAYAHIRRQFKISIGKMEGIEEPLARIAGNAYVMDAAASLIT |
| | | YGIMLGEKPAVLSAIVKYHCTHRGQQSIIDAMDITGGKGIMLGQSNFLARAYQG |
| | | APIAITVEGANILTRSMMIFGQGAIRCHPYVLEEMEAAKNNDVNAFDKLLFKHIG |
| | | HVGSNKVRSFWLGLTRGLTSSTPTGDATKRYYQHLNRLSANLALLSDVSMAVL |
| | | GGSLKRRERISARLGDILSQLYLASAVLKRYDDEGRNEADLPLVHWGVQDALY |
| | | QAEQAMDDLLQNFPNRVVAGLLNVVIFPTGRHYLAPSDKLDHKVAKILQVPNA |
| | | TRSRIGRGQYLTPSEHNPVGLLEEALVDVIAADPIHQRICKELGKNLPFTRLDELA |
| | | HNALVKGLIDKDEAAILVKAEESRLRSINVDDFDPEELATKPVKLPEKVRKVEA |
| | | A |
| 27 | acyl-CoA | MKKVWLNRYPADVPTEINPDRYQSLVDMFEQSVARYADQPAFVNMGEVMTFR |
| | synthetase (FadD) | KLEERSRAFAAYLQQGLGLKKGDRVALMMPNLLQYPVALFGILRAGMIVVNVN |
| | from E. coli | PLYTPRELEHQLNDSGASAIVIVSNFAHTLEKVVDKTAVQHVILTRMGDQLSTA |
| | | KGTVVNFVVKYIKRLVPKYHLPDAISFRSALHNGYRMQYVKPELVPEDLAFLQY |
| | | TGGTTGVAKGAMLTHRNMLANLEQVNATYGPLLHPGKELVVTALPLYHIFALT |
| | | INCLLFIELGGQNLLITNPRDIPGLVKELAKYPFTAITGVNTLFNALLNNKEFQQL |
| | | DFSSLHLSAGGGMPVQQVVAERWVKLTGQYLLEGYGLTECAPLVSVNPYDIDY |
| | | HSGSIGLPVPSTEAKLVDDDDNEVPPGQPGELCVKGPQVMLGYWQRPDATDEII |
| | | KNGWLHTGDIAVMDEEGFLRIVDRKKDMILVSGFNVYPNEIEDVVMQHPGVQE |
| | | VAAVGVPSGSSGEAVKIFVVKKDPSLTEESLVTFCRRQLTGYKVPKLVEFRDELP |
| | | KSNVGKILRRELRDEARGKVDNKA |
| 28 | Acyl-ACP | MATTSLASAFCSMKAVMLARDGRGMKPRSSDLQLRAGNAPTSLKMINGTKFSY |
| | thioesterase | TESLKRLPDWSMLFAVITTIFSAAEKQWTNLEWKPKPKLPQLLDDHFGLHGLVF |
| | (FatB1) from | RRTFAIRSYEVGPDRSTSILAVMNHMQEATLNHAKSVGILGDGFGTTLEMSKRD |
| | Umbellularia | LMWVVRRTHVAVERYPTWGDTVEVECWIGASGNNGMRRDFLVRDCKTGEILT |
| | californica | RCTSLSVLMNTRTRRLSTIPDEVRGEIGPAFIDNVAVKDDEIKKLQKLNDSTADYI |
| | (UniProt_Q41635) | QGGLTPRWNDLDVNQHVNNLKYVAWVFETVPDSIFESHHISSFTLEYRRECTRD |
| | | SVLRSLTTVSGGSSEAGLVCDHLLQLEGGSEVLRARTEWRPKLTDSFRGISVIPA |
| | | EPRV |
| 29 | ester synthase from | MARNIPLLDASWLYVESKEAPMHVGSMAIFTVPEGETSQQAIARIVQMLRNSLE |
| | Limnobacter sp. | FAPPFNYRLSSPRLLTLMPKWIEADKIDLDYHFRHSALPAPGGERELGTLISRLHS |
| | (UniProt_A6GSQ9) | HPLDFRKPLWEMHLIEGLYGNRFALYTKMHHSLMDGVGGMRLMERIFGKSAK |
| | | ESMNLPAPWSVGTISRKKKNSEPQHFADQAREAWEAAKLSGQSLPAAGRALMD |
| | | LMREAVKPTDPALATPFSGPKSIVNKRVGGARRLATQTYPLERVRAVAEAAKVS |
| | | VNDIFLAICSSSIRRYLLERDALPSESLTAGLPVSVRPADDLDGGNAISFIIANLYT |
| | | TEADPLTRLKEIRRSTQLAKANLQAMPKEAINNYTIMLMAPMMLQLVSGLGGLT |
| | | RPIFNTVISNVPGPSRDLYFSGCRLEQFYPISLIPHGQALNITVVSYSGQFNVAFTG |
| | | DHDALPSMQRLSVYTGEALEELEAALGVKWASKPVVKPVTEKRPVAAKKPAV |
| | | RKPATAKVGAGKPVKAPED |
| 30 | β-keto-acyl-ACP | MKRAVITGLGIVSSIGNNQQEVLASLREGRSGITFSQELKDSGMRSHVWGNVKL |
| | synthase I from E. | DTTGLIDRKVVRFMSDASIYAFLSMEQAIADAGLSPEAYQNNPRVGLIAGSGGGS |
| | coli (FabB, | PRFQVFGADAMRGPRGLKAVGPYVVTKAMASGVSACLATPFKIHGVNYSISSA |
| | UniProt_POA953) | CATSAHCIGNAVEQIQLGKQDIVFAGGGEELCWEMACEFDAMGALSTKYNDTP |
| | | EKASRTYDAHRDGFVIAGGGGMVVVEELEHALARGAHIYAEIVGYGATSDGAD |
| | | MVAPSGEGAVRCMKMAMHGVDTPIDYLNSHGTSTPVGDVKELAAIREVFGDKS |
| | | PAISATKAMTGHSLGAAGVQEAIYSLLMLEHGFIAPSINIEELDEQAAGLNIVTET |
| | | TDRELTTVMSNSFGFGGTNATLVMRKLKD |
| 31 | transcriptional | MVIKAQSPAGFAEEYIIESIWNNRFPPGTILPAERELSELIGVTRTTLREVLQRLAR |
| | regulator (FadR) | DGWLTIQHGKPTKVNNFWETSGLNILETLARLDHESVPQLIDNLLSVRTNISTIFI |
| | from E. coli | RTAFRQHPDKAQEVLATANEVADHADAFAELDYNIFRGLAFASGNPIYGLILNG |
| | | MKGLYTRIGRHYFANPEARSLALGFYHKLSALCSEGAHDQVYETVRRYGHESG |
| | | EIWHRMQKNLPGDLAIQGR |
| 32 | acyl-CoA | MLQTRIIKPAEGAYAYPLLIKRLLMSGSRYEKTREIVYRDQMRLTYPQLNERIAR |
| | synthetase | LANVLTEAGVKAGDTVAVMDWDSHRYLECMFAIPMIGAVVHTINVRLSPEQIL |
| | (medium-chain- | YTMNHAEDRVVLVNSDFVGLYQAIAGQLTTVDKTLLLTDGPDKTAELPGLVGE |
| | fatty-acid CoA | YEQLLAAASPRYDFPDFDENSVATTFYTTGTTGNPKGVYFSHRQLVLHTLAEAS |
| | ligase) from P. | VTGSIDSVRLLGSNDVYMPITPMFHVHAWGIPYAATMLGMKQVYPGRYEPDML |
| | putida (FadD3) | VKLWREEKVTFSHCVPTILQMLLNCPNAQGQDFGGWKIIIGGSSLNRSLYQAAL |
| | (UniProt_Q88PT5) | ARGIQLTAAYGMSETCPLISAAHLNDELQAGSEDERVTYRIKAGVPVPLVEAAIV |
| | | DGEGNFLPADGETQGELVLRAPWLTMGYFKEPEKSEELWQGGWLHTGDVATL |
| | | DGMGYIDIRDRIKDVIKTGGEWVSSLDLEDLISRHPAVREVAVVGVADPQWGER |
| | | PFALLVARDGHDIDAKALKEHLKPFVEQGHINKWAIPSQIALVTEIPKTSVGKLD |
| | | KKRIRQDIVQWQASNSAFLSTL |
| 33 | acyl-ACP | MVAAAASSAFFPVPAPGASPKPGKFGNWPSSLSPSFKPKSIPNGGFQVKANDSAH |
| | thioesterase from | PKANGSAVSLKSGSLNTQEDTSSSPPPRTFLHQLPDWSRLLTAITTVFVKSKRPD |
| | Cuphea hookeriana | MHDRKSKRPDMLVDSFGLESTVQDGLVFRQSFSIRSYEIGTDRTASIETLMNHLQ |
| | (FatB2, | ETSLNHCKSTGILLDGFGRTLEMCKRDLIWVVIKMQIKVNRYPAWGDTVEINTR |
| | UniProt_Q39514) | FSRLGKIGMGRDWLISDCNTGEILVRATSAYAMMNQKTRRLSKLPYEVHQEIVP |
| | | LFVDSPVIEDSDLKVHKFKVKTGDSIQKGLTPGWNDLDVNQHVSNVKYIGWILE |
| | | SMPTEVLETQELCSLALEYRRECGRDSVLESVTAMDPSKVGVRSQYQHLLRLED |
| | | GTAIVNGATEWRPKNAGANGAISTGKTSNGNSVS |
| 34 | alcohol | MTTNVIHAYAAMQAGEALVPYSFDAGELQPHQVEVKVEYCGLCHSDVSVLNN |
| | dehydrogenase | EWHSSVYPVVAGHEVIGTITQLGSEAKGLKIGQRVGIGWTAESCQACDQCISGQ |
| | form Acinetobacter | QVLCTGENTATIIGHAGGFADKVRAGWQWVIPLPDELDPTSAGPLLCGGITVFDP |
| | baylyi(UniProt_Q6 | ILKHQIQAIHHVAVIGIGGLGHMAIKLLKAWGCEITAFSSNPNKTDELKAMGAD |
| | F6R9) | HVVNSRDDAEIKSQQGKFDLLLSTVNVPLNWNAYLNTLAPNGTFHFLGVVMEPI |
| | | PVPVGALLGGAKSLTASPTGSPAALRKLLEFAARKNIAPQIEMYPMSELNEAIER |
| | | LHSGQARYRIVLKADF |
| 35 | alcohol-O-acetyl- | MNEIDEKNQAPVHQECLKEMIQNGHARRMGSVEDLYVALNRQNLYRNFCTYG |
| | transferase from | ELSDYCTRDQLTLALREICLKNPTLLHIVLPTRWPNHENYYRSSEYYSRPHPVHD |
| | Saccharomyces sp. | YISVLQELKLSGVVLNEQPEYSAVMKQILEEFKNSKGSYTAKIFKLTTTLTIPYFG |
| | (UniProt_Q6XBT3) | PTGPSWRLICLPEEHTEKWKKFIFVSNHCMSDGRSSIHFFHDLRDELNNIKTPPKK |
| | | LDYIFKYEEDYQLLRKLPEPIEKVIDFRPPYLFIPKSLLSGFIYNHLRFSSKGVCMR |
| | | MDDVEKTDDVVTEIINISPTEFQAIKANIKSNIQGKCTITPFLHVCWFVSLHKWGK |
| | | FFKPLNFEWLTDIFIPADCRSQLPDDDEMRQMYRYGANVGFIDFTPWISEFDMN |
| | | DNKENFWPLIEHYHEVISEALRNKKHLHGLGFNIQGFVQKYVNIDKVMCDRAIG |
| | | KRRGGTLLSNVGLFNQLEEPDAKYSICDLAFGQFQGSWHQAFSLGVCSTN |
| | | VKGMNIVVASTKNVVGSQESLEELCSIYKALLLGP |
| 36 | Acyl-CoA | MNKKLEALFRENVKGKVALITGASSGIGLTIAKRIAAAGAHVLLVARTQETLEE |
| | reductase from | VKAAIEQQGGQASIFPCDLTDMNAIDQLSQQIMASVDHVDFLINNAGRSIRRAVH |
| | Acinetobacter | ESFDRFHDFERTMQLNYFGAVRLVLNLLPHMIKRKNGQIINISSIGVLANATRFSA |
| | baylyi (UniProtKB | YVASKAALDAFSRCLSAEVLKHKISITSIYMPLVRTPMIAPTKIYKYVPTLSPEEA |
| | Q6F7B8) | ADLIVYAIVKRPKRIATHLGRLASITYAIAPDINNILMSIGFNLFPSSTAALGEQEK |
| | | LNLLQRAYARLFPGEHW |
| 37 | Acyl-CoA | MNYFLTGGTGFIGRFLVEKLLARGGTVYVLVREQSQDKLERLRERWGADDKQV |
| | reductase from | KAVIGDLTSKNLGIDAKTLKSLKGNIDHVFHLAAVYDMGADEEAQAATNIEGTR |
| | Marinobacter sp. | AAVQAAEAMGAKHFHHVSSIAAAGLFKGIFREDMFEEAEKLDHPYLRTKHESE |
| | | KVVREECKVPFRIYRPGMVIGHSETGEMDKVDGPYYFFKMIQKIRHALPQWVPT |
| | | IGIEGGRLNIVPVDFVVDALDHIAHLEGEDGNCFHLVDSDPYKVGEILNIFCEAG |
| | | HAPRMGMRIDSRMFGFIPPFIRQSIKNLPPVKRITGALLDDMGIPPSVMSFINYPTR |
| | | FDTRELERVLKGTDIEVPRLPSYAPVIWDYWERNLDPDLFKDRTLKGTVEGKVC |
| | | VVTGATSGIGLATAEKLAEAGAILVIGARTKETLDEVAASLEAKGGNVHAYQCD |
| | | FSDMDDCDRFVKTVLDNHGHVDVLVNNAGRSIRRSLALSFDRFHDFERTMQLN |
| | | YFGSVRLIMGFAPAMLERRRGHVVNISSIGVLTNAPRFSAYVSSKSALDAFSRCA |
| | | AAEWSDRNVTFTTINMPLVKTPMIAPTKIYDSVPTLTPDEAAQMVADAIVYRPK |
| | | RIATRLGVFAQVLHALAPKMGEIIMNTGYRMFPDSPAAAGSKSGEKPKVSTEQV |
| | | AFAAIMRGIYW |
| 38 | acetoacetyl-CoA | MTQRIAYVTGGMGGIGTAICQRLAKDGFRVVAGCGPNSPRREKWLEQQKALG |
| | reductase / phaB | FDFIASEGNVADWDSTKTAFDKVKSEVGEVDVLINNAGITRDVVFRKMTRAD |
| | from Cupriavidus | WDAVIDTNLTSLFNVTKQVIDGMADRGWGRIVNISSVNGQKGQFGQTNYSTA |
| | necator | KAGLHGFTMALAQEVATKGVTVNTVSPGYIATDMVKAIRQDVLDKIVATIPVK |
| | | RLGLPEEIASICAWLSSEESGFSTGADFSLNGGLHMG |
| 39 | 3-hydroxybutyryl- | MSIRTVGIVGAGTMGNGIAQACAVVGLNVVMVDISDAAVQKGVATVAGSLDR |
| | CoA | LIKKEKLTEADKASALARIKGSTSYDDLKATDIVIEAATENYDLKVKILKQIDAIV |
| | dehydrogenase / | GENVIIASNTSSISITKLAAVTSRADRFIGMHFFNPVPVMALVELIRGLQTSDITHA |
| | hbd from | AVEALSKQLGKYPITVRNSPGFVVNRILCPMINEAFCVLGEGLASPEEIDEGMKL |
| | Cupriavidus | GCNHPIGPLALADMIGLDTMLAVMEVLYTEFADPKYRPAMLMREMVAAGYLG |
| | necator | RKTGRGVYVYSK |
| 40 | 3-hydroxyacyl- | MSIRTVGIVGAGTMGNGIAQACAVVGLNVVMVDISDAAVQKGVATVASSLDR |
| | CoA | LIKKEKLTEADKASALARIKGSTSYDDLKATDIVIEAATENYDLKVKILKQIDGI |
| | dehydrogenase / | VGENVIIASNTSSISITKLAAVTSRADRFIGMHFFNPVPVMALVELIRGLQTSDTT |
| | paaHl from | HAAVEALSKQLGKYPITVKNSPGFVVNRILCPMINEAFCVLGEGLASPEEIDEG |
| | Cupriavidus | MKLGCNHPIGPLALADMIGLDTMLAVMEVLYTEFADPKYRPAMLMREMVAA |
| | necator | GYLGRKTGRGVYVYSK |
| 41 | Putative 3-oxoacyl- | MKLQGRVAIITGAAAGIGFATAQRFAEDGAIVVLCDVQEARVREAAARLAATG |
| | [acyl-carrier- | ATVSAYRVDVTRRDEVDAMVAAVLAAHQRVDILVNNAGITKDARLAKMTEAQ |
| | protein] reductase | FDAVIDVNLKGVFNCAQAVAGLMTEQGKGVILNASSVVGLYGNFGQTNYAAS |
| | from Cupriavidus | KFGVIGFTKTWARELGPKGVRVNAVCPGFVNTEILQTVPDKVLDGMTSSCWLR |
| | taiwanensis | RLAEPAEIASIYAFLASDDASYVNGVAIEASGGMSL |
| 42 | 3-oxoacyl-ACP | MSRLQDKVAIITGAANGIGLEAARVFMKEGAKVVIADFNEAAGKEAVEANPGV |
| | reductase (FabG) / | VFIRVDVSDRESVHRLVENVAERFGKIDILINNAGITRDSMLSKMTVDQFQQVIN |
| | fabG from Bacillus | VNLTGVFHCTQAVLPYMAEQGKGKIINTSSVTGTYGNVGQTNYAAAKAGVIGM |
| | sp. SG-1 | TKTWAKELARKGINVNAVAPGFTETAMVAEVPEKVIEKMKAQVPMGRLGKPE |
| | | DIANAYLFLASHESDYVNGHVLHVDGGIMM |
| 43 | 3-ketoacyl-ACP | MSQRIAYVTGGMGGIGTSICQRLSKDGFKVVAGCGPNSPRRVKWLEEQKALGF |
| | reductase from | DFIASEGNVGDWDSTKAAFDKVKAEVGEVDVLVNNAGITRDVVFRKMTHEDW |
| | Burkholderia | TAVIDTNLTSLFNVTKQVIDGMVERGWGRIINISSVNGQKGQFGQTNYSTAKAGI |
| | multivorans | HGFTMALAQEVATKGVTVNTVSPGYIGTDMVKAIRPDVLEKIVATIPVRRLGTP |
| | | EEIGSIVAWLASNDSGFATGADFSLNGGLHMG |
| 44 | acetoacetyl-CoA | MTKRIAYVTGGMGGIGTSICQRLHKDGYTVVAGCGPNSPRRVKWLEEQKANGY |
| | reductase / phbB | DFIASEGNVGDWESTKNAFDKVKAEVGEVDILVNNAGITRDVVFRKMTHEDWT |
| | from Pseudomonas | AVIDTNLTSLFNVTKQVVDGMVERGFGRIINISSVNGQKGQFGQTNYSTAKAGIH |
| | putida | GFTMALAQEVATKGVTVNTVSPGYIGTDMVKAIRPEVLEKIVATIPVRRLGRPD |
| | | EIGSIVSWLASEESGFSTGADFSLNGGLHMG |
| 45 | 3-hydroxybutyryl- | MSKVMVIGAGTMGSGIAQVFATHGWDVVLNDIKDEFIAGGKAKIEKQLTKQVS |
| | CoA | KGKMTQEDKDATMARISGFVTRTPDNMKDIDLVVEAAIEDLKIKSAIFKELDEFC |
| | dehydrogenase / | PEHTILASNTSSLAITQIGAATKRPDKVIGMHFFNPAPVMKLIEVINGIATSEETYD |
| | hbd (MELS_1448) | KIDKLSKDLGKTPVKVNDFPGFAGNRIVIPMLNEAIQALMEGVASKEDIDNVCK |
| | from Megasphaera | LGFNHPMGPLALCDLIGNDVVLHVMEVLYDGFGDPKYRPCALLKKYVQAGYL |
| | elsdenii | GRKTGKGFYDYTK |

What is claimed is:

1. A recombinant microbe, comprising:
i) an acyl-ACP thioesterase and an acyl-CoA synthetase; and
ii) a heterologous R-3-hydroxy acyl-CoA dehydratase or a heterologous R-specific enoyl-CoA hydratase;
wherein the recombinant microbe produces a fatty acid derivative composition comprising fatty acids and derivatives thereof with a reduced amount of 3-hydroxy fatty acids and 3-hydroxy fatty acid derivatives, as compared to a corresponding microbe that does not comprise a heterologous R-3-hydroxy acyl-CoA dehydratase or a heterologous R-specific enoyl-CoA hydratase.

2. The recombinant microbe of claim 1, further comprising a heterologous trans-2-enoyl-CoA reductase.

3. The recombinant microbe of claim 1 or claim 2, wherein:
the recombinant microbe does not produce any 3-hydroxy fatty acids or 3-hydroxy fatty acid derivatives thereof; or
the recombinant microbe produces a fatty acid derivative composition comprising a reduced amount of, or that is free or substantially free of, 3-hydroxy fatty acids and derivatives thereof.

4. The recombinant microbe of any one of claims 1-3, wherein the heterologous R-3-hydroxy-acyl-CoA dehydratase or the heterologous R-specific enoyl-CoA hydratase is PhaJ1, PhaJ3, PhaJ4, MaoC, MaoC9, or an MaoC family dehydratase.

5. The recombinant microbe of any one of claims 1-4, wherein the heterologous R-3-hydroxy-acyl-CoA dehydratase or the heterologous R-specific enoyl-CoA hydratase comprises an amino acid sequence set forth in any one of SEQ ID NOs: 1-13, or is a homolog thereof, or comprises an amino acid sequence having at least 30% sequence identity to any one of SEQ ID NOs: 1-13.

6. The recombinant microbe of any one of claims 1-5, wherein the heterologous R-3-hydroxy-acyl-CoA dehydratase or the heterologous R-specific enoyl-CoA hydratase has activity to convert a 3-hydroxy-acyl-CoA to a corresponding trans-2-enoyl-CoA.

7. The recombinant microbe of any one of claims 2-6, wherein the heterologous trans-2-enoyl-CoA reductase is TER or FabV from Treponema denticola, Euglena gracilis, Enterovibrio coralii, Lactobacillus oligofermentans, Paucilactobacillus oligofermentans, Vibrio spp., Treponema leithinolyticum, Treponema pedis, Treponema vincentii, Treponema sp., Treponema brennaborense, or Treponema parvum.

8. The recombinant microbe of any one of claims 2-7, wherein the heterologous trans-2-enoyl-CoA reductase comprises an amino acid sequence set forth in any one of SEQ ID NOs: 14-25, or is a homolog thereof, or comprises an amino acid sequence having at least 30% sequence identity to any one of SEQ ID NOs: 14-25.

9. The recombinant microbe of any one of claims 2-8, wherein the trans-2-enoyl-CoA reductase has activity to convert a trans-2-enoyl-CoA to a corresponding acyl-CoA.

10. The recombinant microbe of any one of claims 1-9, further comprising one or more enzymes corresponding to an ester synthase, a β-keto-acyl-ACP synthase, an alcohol dehydrogenase, an alcohol-O-acetyl-transferase, a fatty-alcohol-forming acyl-CoA reductase, or an acyl-CoA reductase.

11. The recombinant microbe of any one of claims 1-9, further comprising one or more enzymes corresponding to an ester synthase, a β-keto-acyl-ACP synthase, an alcohol dehydrogenase, an alcohol-O-acetyl-transferase, a fatty-alcohol-forming acyl-CoA reductase, an acyl-CoA reductase, an acyl-CoA thioesterase, a carboxylic acid reductase, a desaturase, an omega-hydroxylase, a transaminase (or aminotransferase), an amine dehydrogenase, a CoA-ligase/transferase, an aldehyde decarbonylase, an aldehyde oxidative deformylase, a decarboxylase, one or more subunits of an acetyl-CoA carboxylase (AccABCD), an OleA, an OleBCD, an OleABCD, an OleACD, or an aldehyde dehydrogenase.

12. The recombinant microbe of any one of claims 1-11, further expressing or overexpressing FadR.

13. The recombinant microbe of any one of claims 1-12, comprising an attenuated or deleted acyl-CoA dehydrogenase.

14. The recombinant microbe of claim 13, wherein the acyl-CoA dehydrogenase is FadE.

15. The recombinant microbe of any one of claims 1-14, wherein acyl-ACP thioesterase is FatB1 from Umbellularia californica or is FatB2 from Cuphea hookeriana.

16. The recombinant microbe of any one of claims 1-15, wherein the acyl-CoA synthetase is FadD from E. coli, or is FadD3 from Pseudomonas putida.

17. The recombinant microbe of any one of claims 1-16, wherein the heterologous R-specific enoyl-CoA hydratase is PhaJ1 from Pseudomonas putida, PhaJ4 from Pseudomonas putida, PhaJ3 from Pseudomonas aeruginosa, or PhaJ4 from Pseudomonas aeruginosa.

18. The recombinant microbe of any one of claims 1-17, wherein the recombinant microbe produces a fatty acid derivative, or a composition comprising a fatty acid derivative, wherein the fatty acid derivative is a fatty ester, a fatty alcohol, a fatty alcohol acetate ester, a fatty aldehyde, a fatty amine, a fatty amide, a fatty diol, a fatty triol, a fatty tetrol, an ω-hydroxy fatty acid, an ω-carboxy fatty acid, an ω-hydroxy fatty ester, an ω-carboxy fatty ester, an α,ω-fatty diacid, an α,ω-fatty diester, or an α,ω-fatty diol, or a combination thereof.

19. The recombinant microbe of any one of claims 1-18, wherein the recombinant microbe produces a reduced amount of a 3-hydroxy fatty acid or derivative thereof, compared to a corresponding microbe that does not comprise a heterologous R-3-hydroxy-acyl-CoA dehydratase or a heterologous R-specific enoyl-CoA hydratase.

20. The recombinant microbe of any one of claims 2-18, wherein:
the recombinant microbe produces a reduced amount of a 3-hydroxy fatty acid or derivative thereof; or
the recombinant microbe produces a reduced amount of a 3-hydroxy fatty acid or derivative thereof and a reduced amount of a trans-2-fatty acid or derivative thereof;
compared to a corresponding microbe that does not comprise a heterologous R-3-hydroxy-acyl-CoA dehydratase or a heterologous R-specific enoyl-CoA hydratase, and does not comprise a heterologous trans-2-enoyl-CoA reductase.

21. The recombinant microbe of claim 19 or claim 20, wherein:
the 3-hydroxy fatty acid or derivative thereof is a 3-hydroxy fatty acid; a 3-hydroxy fatty acid ester; a 1,3-diol; a fatty alcohol 1,3-diacetate; a 3-hydroxy fatty aldehyde; a 3-hydroxy fatty amine; a 3-hydroxy fatty amide; a fatty diol with a 3-hydroxy group; a fatty triol with a 3-hydroxy group; a fatty tetrol with a 3-hydroxy group; an ω-hydroxy fatty acid with a 3-hydroxy group, an ω-carboxy fatty acid with a 3-hydroxy group, an ω-hydroxy fatty ester with a 3-hydroxy group, an ω-carboxy fatty ester with a 3-hydroxy group, an α,ω-fatty diacid with a 3-hydroxy group, an α,ω-fatty diester with a 3-hydroxy group, or an α,ω-fatty diol with a 3-hydroxy group, or a combination thereof; and
the trans-2-fatty acid or derivative thereof is a trans-2-fatty acid; a trans-2-fatty acid ester; a trans-2-fatty alcohol; a trans-2-fatty alcohol acetate; a trans-2-fatty aldehyde; a trans-2-fatty amine; a trans-2-fatty amide; a trans-2-fatty 1,3-diol; a trans-2-fatty diol; a trans-2-fatty triol; a trans-2-fatty tetrol; a trans-2-ω-hydroxy fatty acid, a trans-2-ω-carboxy fatty acid, a trans-2-ω-hydroxy fatty ester; a trans-2-ω-carboxy fatty ester; a trans-2-α,ω-fatty diacid; a trans-2-α,ω-fatty diester; or a trans-2-α,ω-fatty diol, or a combination thereof.

22. The recombinant microbe of any one of claims 1-21, further comprising a heterologous 3-oxoacyl-CoA reductase, a heterologous β-ketoacyl-CoA-reductase, or a heterologous 3-hydroxy acyl-CoA dehydrogenase.

23. The recombinant microbe of claim 22, wherein the recombinant microbe produces a reduced amount of 3-hydroxy fatty acids or derivatives thereof, and produces a reduced amount of 3-oxo fatty acids or derivatives thereof, compared to a corresponding microbe that does not comprise a heterologous R-3-hydroxy acyl-CoA dehydratase, a heterologous R-specific enoyl-CoA hydratase, a heterologous 3-oxoacyl-CoA reductase, a heterologous β-ketoacyl-CoA-reductase, or a heterologous 3-hydroxy acyl-CoA dehydrogenase.

24. The recombinant microbe of any one of claims 2-21, further comprising a heterologous 3-oxoacyl-CoA reductase, a heterologous β-ketoacyl-CoA-reductase, or a heterologous 3-hydroxy acyl-CoA dehydrogenase, wherein the recombinant microbe produces a reduced amount of 3-hydroxy fatty acids or derivatives thereof, a reduced amount of 3-oxo fatty acids or derivatives thereof, and/or a reduced amount of trans-2-fatty acids and derivatives thereof, compared to a corresponding microbe that does not comprise a heterologous R-3-hydroxy acyl-CoA dehydratase, a heterologous R-specific enoyl-CoA hydratase, a heterologous trans-2-enoyl-CoA reductase, a heterologous 3-oxoacyl-CoA reductase, a heterologous β-ketoacyl-CoA-reductase, or a heterologous 3-hydroxy acyl-CoA dehydrogenase.

25. The recombinant microbe of any one of claims 22-24, wherein the 3-oxoacyl-CoA reductase, β-ketoacyl-CoA-reductase, or 3-hydroxy acyl-CoA dehydrogenase is PhaB, HbD, PaaH1, FabG, PhhB, or MELS_1448.

26. The recombinant microbe of any one of claims 22-25, wherein the 3-oxoacyl-CoA reductase, β-ketoacyl-CoA-reductase, or 3-hydroxy acyl-CoA dehydrogenase is from Cupriavidus necator, Ralstonia eutropha, Cupriavidus necator H16, Cupriavidus taiwanensis, Bacillus sp. SG-1, Burkholderia multivorans, Pseudomonas putida, or Megasphaera elsdenii.

27. The recombinant microbe of any one of claims 22-26, wherein the 3-oxoacyl-CoA reductase, β-ketoacyl-CoA-reductase, or 3-hydroxy acyl-CoA dehydrogenase comprises an amino acid sequence set forth in any one of SEQ ID NOs: 38-45, or is a homolog thereof, or comprises an amino acid sequence having at least 30% sequence identity to any one of SEQ ID NOs: 38-45.

28. The recombinant microbe of any one of claims 22-27, wherein the 3-oxoacyl-CoA reductase, β-ketoacyl-CoA-reductase, or 3-hydroxy acyl-CoA dehydrogenase has activity to convert a 3-oxo-acyl-CoA to a corresponding 3-hydroxy-acyl-CoA.

29. The recombinant microbe of any one of claims 22-28, wherein the recombinant microbe produces a fatty acid derivative, or a composition comprising a fatty acid derivative, wherein the fatty acid derivative is a fatty ester, a fatty alcohol, a fatty alcohol acetate ester, a fatty aldehyde, a fatty amine, a fatty amide, a fatty diol, a fatty triol, a fatty tetrol, an ω-hydroxy fatty acid, an ω-carboxy fatty acid, an ω-hydroxy fatty ester, an ω-carboxy fatty ester, an α,ω-fatty diacid, an α,ω-fatty diester, or an α,ω-fatty diol, or a combination thereof.

30. The recombinant microbe of any one of claims 23-29, wherein:
the 3-hydroxy fatty acid or derivative thereof is a 3-hydroxy fatty acid; a 3-hydroxy fatty acid ester; a 1,3-diol; a fatty alcohol 1,3-diacetate; a 3-hydroxy fatty aldehyde; a 3-hydroxy fatty amine; a 3-hydroxy fatty amide; a fatty diol with a 3-hydroxy group; a fatty triol with a 3-hydroxy group; a fatty tetrol with a 3-hydroxy group; an ω-hydroxy fatty acid with a 3-hydroxy group, an ω-carboxy fatty acid with a 3-hydroxy group, an ω-hydroxy fatty ester with a 3-hydroxy group, an ω-carboxy fatty ester with a 3-hydroxy group, an α,ω-fatty diacid with a 3-hydroxy group, an α,ω-fatty diester with a 3-hydroxy group, or an α,ω-fatty diol with a 3-hydroxy group, or a combination thereof; and
the 3-oxo fatty acid or derivative thereof is a 3-oxo fatty acid; a 3-oxo fatty acid ester; a 3-oxo fatty alcohol; a 3-oxo fatty alcohol acetate ester; a 3-oxo fatty aldehyde; a 3-oxo fatty amine; a 3-oxo fatty amide; a 3-oxo fatty diol; a 3-oxo fatty triol; a 3-oxo fatty tetrol; an ω-hydroxy fatty acid with a 3-oxo group, an ω-carboxy fatty acid with a 3-oxo group, an ω-hydroxy fatty ester with a 3-oxo group, an ω-carboxy fatty ester with a 3-oxo group, an α,ω-fatty diacid with a 3-oxo group, an α,ω-fatty diester with a 3-oxo group, or an α,ω-fatty diol with a 3-oxo group, or a combination thereof.

31. The recombinant microbe of any one of claims 24-29, wherein:
the 3-hydroxy fatty acid or derivative thereof is a 3-hydroxy fatty acid; a 3-hydroxy fatty acid ester; a 1,3-diol; a fatty alcohol 1,3-diacetate; a 3-hydroxy fatty aldehyde; a 3-hydroxy fatty amine; a 3-hydroxy fatty amide; a fatty diol with a 3-hydroxy group; a fatty triol with a 3-hydroxy group; a fatty tetrol with a 3-hydroxy group; an ω-hydroxy fatty acid with a 3-hydroxy group, an ω-carboxy fatty acid with a 3-hydroxy group, an ω-hydroxy fatty ester with a 3-hydroxy group, an ω-carboxy fatty ester with a 3-hydroxy group, an α,ω-fatty diacid with a 3-hydroxy group, an α,ω-fatty diester with a 3-hydroxy group, or an α,ω-fatty diol with a 3-hydroxy group, or a combination thereof;
the 3-oxo fatty acid or derivative thereof is a 3-oxo fatty acid; a 3-oxo fatty acid ester; a 3-oxo fatty alcohol; a 3-oxo fatty alcohol acetate ester; a 3-oxo fatty aldehyde; a 3-oxo fatty amine; a 3-oxo fatty amide; a 3-oxo fatty diol; a 3-oxo fatty triol; a 3-oxo fatty tetrol; an ω-hydroxy fatty acid with a 3-oxo group, an ω-carboxy fatty acid with a 3-oxo group, an ω-hydroxy fatty ester with a 3-oxo group, an ω-carboxy fatty ester with a 3-oxo group, an α,ω-fatty diacid with a 3-oxo group, an α,ω-fatty diester with a 3-oxo group, or an α,ω-fatty diol with a 3-oxo group, or a combination thereof; and
the trans-2-fatty acid or derivative thereof is a trans-2-fatty acid; a trans-2-fatty acid ester; a trans-2-fatty alcohol; a trans-2-fatty alcohol acetate; a trans-2-fatty aldehyde; a trans-2-fatty amine; a trans-2-fatty amide; a trans-2-fatty 1,3-diol; a trans-2-fatty diol; a trans-2-fatty triol; a trans-2-fatty tetrol; a trans-2-ω-hydroxy fatty acid, a trans-2-ω-carboxy fatty acid, a trans-2-ω-hydroxy fatty ester; a trans-2-ω-carboxy fatty ester; a trans-2-α,ω-fatty diacid; a trans-2-α,ω-fatty diester; or a trans-2-α,ω-fatty diol, or a combination thereof.

32. The recombinant microbe of any one of claims 1-31, wherein the acyl-ACP thioesterase has activity for the conversion of a 3-hydroxy-acyl-ACP to a corresponding 3-hydroxy fatty acid, and/or has activity for the conversion of a 3-keto-acyl-ACP to a corresponding 3-oxo-fatty acid.

33. The recombinant microbe of any one of claims 1-32, wherein the acyl-CoA synthetase has activity for the conversion a 3-hydroxy fatty acid to a corresponding 3-hydroxy-acyl-CoA, and/or have activity for the conversion of a 3-oxo fatty acid to a corresponding 3-oxo-acyl-CoA.

34. The recombinant microbe of any one of claims 1-33, wherein the recombinant microbe is a bacterium, a cyanobacterium, a yeast, or an algae.

35. The recombinant microbe of claim 34, wherein the recombinant microbe is a recombinant γ-proteobacterium.

36. The recombinant microbe of claim 35, wherein the recombinant y-proteobacterium is Escherichia coli, Salmonella spp., Vibrio natriegens, Pseudomonas aeruginosa, Pseudomonas putida, Pseudomonas fluorescens, Xanthomonas axonopodis, Pseudomonas syringae, Xyella fastidiosa, Marinobacter aquaeolei, Yersinia pestis, Bacillus spp., Lactobacillus spp., Zymomonas spp., Streptomyces spp., or Vibrio cholerae.

37. The recombinant microbe of claim 35, wherein the recombinant γ-proteobacterium is Escherichia coli.

38. The recombinant microbe of claim 34, wherein the recombinant microbe is a cyanobacterium selected from Synechococcus elongatus PCC7942 and Synechocystis sp. PCC6803.

39. The recombinant microbe of claim 34, wherein:
the recombinant microbe is a yeast selected from Saccharomyces cerevisiae, Scheffersomyces stipites, Schizosaccharomyces pombe, Kluyveromyces marxianus, K. lactis, Pichia pastoris, Hansenula polymorpha, and Yarrowia lipolytica; or
the recombinant microbe is an algae selected from Botryococcus braunii, Nannochloropsis gaditina, Chlamydomonas reinhardtii, Chlorella vulgaris, Spirulina platensis, Ostreococcus tauri, Phaeodactylum tricornutum, Symbiodinium sp., phytoplanktons, cyanobacterial mats, Saccharina japonica, Chlorococum spp., and Spirogyra spp.

40. The recombinant microbe of any one of claims 1-39, comprising an acyl-ACP dependent fatty acid biosynthetic pathway for the production of fatty acids and derivatives thereof.

41. A cell culture, comprising the recombinant microbe of any one of claims 1-40.

42. A method for producing a fatty acid or derivative thereof, the method comprising culturing the recombinant microbe of any one of claims 1-40, or the cell culture of claim 41, in the presence of a carbon source.

43. The method of claim 42, further comprising isolating the fatty acid or derivative thereof.

44. The method of claim 42 or claim 43, wherein the recombinant microbe produces a reduced amount of, or does not produce any of, i) 3-hydroxy fatty acids and derivatives thereof, or ii) trans-2-fatty acids and derivatives thereof, or a combination thereof,
compared to a corresponding microbe that does not express a heterologous R-3-hydroxy acyl-CoA dehydratase or a heterologous R-specific enoyl-CoA hydratase, and does not express a heterologous trans-2-enoyl-CoA reductase.

45. The method of claim 42 or claim 43, wherein the recombinant microbe produces a reduced amount of, or does not produce any of, one or more of i) 3-hydroxy fatty acids and derivatives thereof; ii) trans-2-fatty acids and derivatives thereof; and/or iii) 3-oxo fatty acids and derivatives thereof;
compared to a corresponding microbe that does not express i) a heterologous R-3-hydroxy acyl-CoA dehydratase or a heterologous R-specific enoyl-CoA hydratase; ii) a heterologous trans-2-enoyl-CoA reductase; and/or iii) a heterologous 3-oxoacyl-CoA reductase, a heterologous β-ketoacyl-CoA-reductase, or a heterologous 3-hydroxy acyl-CoA dehydrogenase.

46. A method for preparing a fatty acid derivative composition, the method comprising culturing the recombinant microbe of any one of claims 1-40, or the cell culture of claim 41, in the presence of a carbon source, wherein:
the fatty acid derivative composition comprises a fatty acid or derivative thereof; and
the fatty acid derivative composition comprises a reduced amount of, or is free of, or is substantially free of, i) 3-hydroxy fatty acids and derivatives thereof, or ii) trans-2-fatty acids and derivatives thereof, or a combination thereof, compared to a fatty acid derivative composition prepared by culturing a recombinant microbe that does not express a heterologous R-3-hydroxy acyl-CoA dehydratase or a heterologous R-specific enoyl-CoA hydratase, and does not express a heterologous trans-2-enoyl-CoA reductase.

47. A method for preparing a fatty acid derivative composition, the method comprising culturing the recombinant microbe of any one of claims 1-40, or the cell culture of claim 41, in the presence of a carbon source, wherein:
the fatty acid derivative composition comprises a fatty acid or derivative thereof; and
the fatty acid derivative composition comprises a reduced amount of, or is free of, or is substantially free of, one or more of i) 3-hydroxy fatty acids and derivatives thereof; ii) trans-2-fatty acids and derivatives thereof; and/or iii) 3-oxo fatty acids and derivatives thereof, compared to a corresponding microbe that does not express i) a heterologous R-3-hydroxy acyl-CoA dehydratase or a heterologous R-specific enoyl-CoA hydratase; ii) a heterologous trans-2-enoyl-CoA reductase; and/or iii) a heterologous 3-oxoacyl-CoA reductase, a heterologous β-ketoacyl-CoA-reductase, or a heterologous 3-hydroxy acyl-CoA dehydrogenase.

48. The method of claim 46 or claim 47, further comprising isolating the fatty acid derivative composition.

49. The method of any one of claims 42-48, wherein the fatty acid or derivative thereof is a fatty ester, a fatty alcohol, a fatty alcohol acetate ester, a fatty aldehyde, a fatty amine, a fatty amide, a fatty diol, a fatty triol, a fatty tetrol, an ω-hydroxy fatty acid, an ω-carboxy fatty acid, an ω-hydroxy fatty ester, an ω-carboxy fatty ester, an α,ω-fatty diacid, an α,ω-fatty diester, or an α,ω-fatty diol, or a combination thereof.

50. The method of any one of claims 44-49, wherein:
the 3-hydroxy fatty acid or derivative thereof is a 3-hydroxy fatty acid; a 3-hydroxy fatty acid ester; a 1,3-diol; a fatty alcohol 1,3-diacetate; a 3-hydroxy fatty aldehyde; a 3-hydroxy fatty amine; a 3-hydroxy fatty amide; a fatty diol with a 3-hydroxy group; a fatty triol with a 3-hydroxy group; a fatty tetrol with a 3-hydroxy group; an ω-hydroxy fatty acid with a 3-hydroxy group, an ω-carboxy fatty acid with a 3-hydroxy group, an ω-hydroxy fatty ester with a 3-hydroxy group, an ω-carboxy fatty ester with a 3-hydroxy group, an α,ω-fatty diacid with a 3-hydroxy group, an α,ω-fatty diester with a 3-hydroxy group, or an α,ω-fatty diol with a 3-hydroxy group, or a combination thereof;
the 3-oxo fatty acid or derivative thereof is a 3-oxo fatty acid; a 3-oxo fatty acid ester; a 3-oxo fatty alcohol; a 3-oxo fatty alcohol acetate ester; a 3-oxo fatty aldehyde; a 3-oxo fatty amine; a 3-oxo fatty amide; a 3-oxo fatty diol; a 3-oxo fatty triol; a 3-oxo fatty tetrol; an ω-hydroxy fatty acid with a 3-oxo group, an ω-carboxy fatty acid with a 3-oxo group, an ω-hydroxy fatty ester with a 3-oxo group, an ω-carboxy fatty ester with a 3-oxo group, an α,ω-fatty diacid with a 3-oxo group, an α,ω-fatty diester with a 3-oxo group, or an α,ω-fatty diol with a 3-oxo group, or a combination thereof; and
the trans-2-fatty acid or derivative thereof is a trans-2-fatty acid; a trans-2-fatty acid ester; a trans-2-fatty alcohol; a trans-2-fatty alcohol acetate; a trans-2-fatty aldehyde; a trans-2-fatty amine; a trans-2-fatty amide; a trans-2-fatty 1,3-diol; a trans-2-fatty diol; a trans-2-fatty triol; a trans-2-fatty tetrol; a trans-2-ω-hydroxy fatty acid, a trans-2-ω-carboxy fatty acid, a trans-2-ω-hydroxy fatty ester; a trans-2-ω-carboxy fatty ester; a trans-2-α,ω-fatty diacid; a trans-2-α,ω-fatty diester; or a trans-2-α,ω-fatty diol, or a combination thereof.

51. The method of any one of claims 42-50, further comprising adding an alcohol to the culture or medium to produce a fatty acid alkyl ester.

52. The method of claim 51, wherein:
the alcohol is methanol, and the fatty acid alkyl ester is a fatty acid methyl ester (FAME); or
the alcohol is ethanol, and the fatty acid alkyl ester is a fatty acid ethyl ester (FAEE).

53. The method of claim 52, wherein the fatty acid alkyl ester is one or more of dodecanoic acid ethyl ester, dodecanoic acid methyl ester, decanoic acid ethyl ester, decanoic acid methyl ester, octanoic acid ethyl ester, and octanoic methyl ester.

54. A fatty acid derivative, or a fatty acid derivative composition, prepared by the method of any one of claims 42-53.

55. The fatty acid derivative or the fatty acid derivative composition of claim 54, wherein the fatty acid derivative or fatty acid derivative composition is purified.

56. The fatty acid derivative or the fatty acid derivative composition of claim 54 or claim 55,
wherein the fatty acid derivative or the fatty acid derivative composition is purified by a two-step centrifugation and water-washing; decanting centrifugation and solvent extraction from a biomass; or whole broth extraction with a water immiscible solvent; or a combination thereof.

57. A composition, comprising a fatty acid or derivative thereof, wherein:
the composition comprises a reduced amount of i) 3-hydroxy fatty acids and derivatives thereof; ii) trans-2-fatty acids and derivatives thereof; or iii) 3-oxo fatty acids and derivatives thereof; or a combination thereof; or
the composition is free or substantially free of i) 3-hydroxy fatty acids and derivatives thereof; ii) trans-2-fatty acids and derivatives thereof; or iii) 3-oxo fatty acids and derivatives thereof; or a combination thereof.

58. The composition of claim 57, wherein:
the fatty acid or derivative thereof is a fatty ester, a fatty alcohol, a fatty alcohol acetate ester, a fatty aldehyde, a fatty amine, a fatty amide, a fatty diol, a fatty triol, a fatty tetrol, an ω-hydroxy fatty acid, an ω-carboxy fatty acid, an ω-hydroxy fatty ester, an ω-carboxy fatty ester, an α,ω-fatty diacid, an α,ω-fatty diester, or an α,ω-fatty diol, or a combination thereof;
the 3-hydroxy fatty acid or derivative thereof is a 3-hydroxy fatty acid; a 3-hydroxy fatty acid ester; a 1,3-diol; a fatty alcohol 1,3-diacetate (1,3-fatty diol acetate); a 3-hydroxy fatty aldehyde; a 3-hydroxy fatty amine; a 3-hydroxy fatty amide; a fatty diol with a 3-hydroxy group;
a fatty triol with a 3-hydroxy group; a fatty tetrol with a 3-hydroxy group; an ω-hydroxy fatty acid with a 3-hydroxy group, an ω-carboxy fatty acid with a 3-hydroxy group, an ω-hydroxy fatty ester with a 3-hydroxy group, an ω-carboxy fatty ester with a 3-hydroxy group, an α,ω-fatty diacid with a 3-hydroxy group, an α,ω-fatty diester with a 3-hydroxy group, or an α,ω-fatty diol with a 3-hydroxy group, or a combination thereof;
the 3-oxo fatty acid or derivative thereof is a 3-oxo fatty acid; a 3-oxo fatty acid ester; a 3-oxo fatty alcohol; a 3-oxo fatty alcohol acetate ester; a 3-oxo fatty aldehyde; a 3-oxo fatty amine; a 3-oxo fatty amide; a 3-oxo fatty diol; a 3-oxo fatty triol; a 3-oxo fatty tetrol; an ω-hydroxy fatty acid with a 3-oxo group, an ω-carboxy fatty acid with a 3-oxo group, an ω-hydroxy fatty ester with a 3-oxo group, an ω-carboxy fatty ester with a 3-oxo group, an α,ω-fatty diacid with a 3-oxo group, an α,ω-fatty diester with a 3-oxo group, or an α,ω-fatty diol with a 3-oxo group, or a combination thereof; and
the trans-2-fatty acid or derivative thereof is a trans-2-fatty acid; a trans-2-fatty acid ester; a trans-2-fatty alcohol; a trans-2-fatty alcohol acetate; a trans-2-fatty aldehyde; a trans-2-fatty amine; a trans-2-fatty amide; a trans-2-fatty 1,3-diol; a trans-2-fatty diol; a trans-2-fatty triol; a trans-2-fatty tetrol; a trans-2-ω-hydroxy fatty acid, a trans-2-ω-carboxy fatty acid, a trans-2-ω-hydroxy fatty ester; a trans-2-ω-carboxy fatty ester; a trans-2-α,ω-fatty diacid; a trans-2-α,ω-fatty diester; or a trans-2-α,ω-fatty diol, or a combination thereof.

59. The composition of claim 57 or claim 58, wherein the composition comprises less than 50%, less than 45%, less than 40%, less than 35%, less than 30%, less than 25%, less than 20%, less than 15%, less than 10%, less than 5%, less than 4%, less than 3%, less than 2%, less than 1%, less than 0.5%, or less, by weight of the composition, of i) 3-hydroxy fatty acids and derivatives thereof; ii) trans-2-fatty acids and derivatives thereof; or iii) 3-oxo fatty acids and derivatives thereof; or a combination thereof.

60. A modified biosynthetic pathway, comprising:
i) an acyl-ACP thioesterase and an acyl-CoA synthetase; and
ii) a heterologous R-3-hydroxy acyl-CoA dehydratase or a heterologous R-specific enoyl-CoA hydratase,
wherein the modified biosynthetic pathway produces a fatty acid derivative composition comprising fatty acids and derivatives thereof, and comprising a reduced amount of 3-hydroxy fatty acids and 3-hydroxy fatty acid derivatives, as compared to a corresponding biosynthetic pathway that does not comprise a heterologous R-3-hydroxy acyl-CoA dehydratase or a heterologous R-specific enoyl-CoA hydratase.

61. A modified biosynthetic pathway, comprising:
i) an acyl-ACP thioesterase and an acyl-CoA synthetase;
ii) a heterologous R-3-hydroxy acyl-CoA dehydratase or a heterologous R-specific enoyl-CoA hydratase; and
iii) a heterologous trans-2-enoyl-CoA reductase,
wherein the modified biosynthetic pathway produces a fatty acid derivative composition comprising fatty acids and derivatives thereof, and comprising a reduced amount of 3-hydroxy fatty acids and derivatives thereof, as compared to a corresponding biosynthetic pathway that does not comprise a heterologous R-3-hydroxy acyl-CoA dehydratase or a heterologous R-specific enoyl-CoA hydratase, and a heterologous trans-2-enoyl-CoA reductase.

62. The modified biosynthetic pathway of claim 60 or claim 61, wherein the fatty acid derivative composition is free or substantially free of 3-hydroxy fatty acids and derivatives thereof.

63. The modified biosynthetic pathway of any one of claims 60-62, wherein the fatty acid derivative composition further comprises a reduced amount of trans-2-fatty acids and derivatives thereof, or wherein the fatty acid derivative composition is free or substantially free of trans-2-fatty acids and derivatives thereof.

64. A modified biosynthetic pathway, comprising:
i) an acyl-ACP thioesterase and an acyl-CoA synthetase;
ii) a heterologous 3-oxoacyl-CoA reductase, a heterologous β-ketoacyl-CoA-reductase, or a heterologous 3-hydroxy acyl-CoA dehydrogenase; and
iii) a heterologous R-3-hydroxy acyl-CoA dehydratase or a heterologous R-specific enoyl-CoA hydratase,
wherein the modified biosynthetic pathway produces a fatty acid derivative composition comprising fatty acids and derivatives thereof, and comprising a reduced amount of 3-hydroxy fatty acids and 3-hydroxy fatty acid derivatives, and a reduced amount of 3-oxo fatty acids and derivatives thereof, as compared to a corresponding biosynthetic pathway that does not comprise a heterologous 3-oxoacyl-CoA reductase, a heterologous β-ketoacyl-CoA-reductase, or a heterologous 3-hydroxy acyl-CoA dehydrogenase; and/or does not comprise a heterologous R-3-hydroxy acyl-CoA dehydratase or a heterologous R-specific enoyl-CoA hydratase.

65. A modified biosynthetic pathway, comprising:
i) an acyl-ACP thioesterase and an acyl-CoA synthetase;
ii) a heterologous 3-oxoacyl-CoA reductase, a heterologous β-ketoacyl-CoA-reductase, or a heterologous 3-hydroxy acyl-CoA dehydrogenase;
iii) a heterologous R-3-hydroxy acyl-CoA dehydratase or a heterologous R-specific enoyl-CoA hydratase; and
iv) a heterologous trans-2-enoyl-CoA reductase,
wherein the modified biosynthetic pathway produces a fatty acid derivative composition comprising fatty acids and derivatives thereof, and comprising a reduced amount of 3-hydroxy fatty acids and derivatives thereof, and a reduced amount of 3-oxo fatty acids and derivatives thereof, as compared to a corresponding biosynthetic pathway that does not comprise ii), iii), and/or iv).

66. The modified biosynthetic pathway of claim 64 or claim 65, wherein the fatty acid derivative composition is free or substantially free of 3-hydroxy fatty acids and derivatives thereof and is free or substantially free of 3-oxo fatty acids and derivatives thereof.

67. The modified biosynthetic pathway of any one of claims 64-66, wherein the fatty acid derivative composition further comprises a reduced amount of trans-2-fatty acids and derivatives thereof, or wherein the fatty acid derivative composition is free or substantially free of trans-2-fatty acids and derivatives thereof.

68. The modified biosynthetic pathway of any one of claims 60-67, wherein:
the fatty acid or derivative thereof is a fatty ester, a fatty alcohol, a fatty alcohol acetate ester, a fatty aldehyde, a fatty amine, a fatty amide, a fatty diol, a fatty triol, a fatty tetrol, an ω-hydroxy fatty acid, an ω-carboxy fatty acid, an ω-hydroxy fatty ester, an ω-carboxy fatty ester, an α,ω-fatty diacid, an α,ω-fatty diester, or an α,ω-fatty diol, or a combination thereof;
the 3-hydroxy fatty acid or derivative thereof is a 3-hydroxy fatty acid; a 3-hydroxy fatty acid ester; a 1,3-diol; a fatty alcohol 1,3-diacetate (1,3-fatty diol acetate); a 3-hydroxy fatty aldehyde; a 3-hydroxy fatty amine; a 3-hydroxy fatty amide; a fatty diol with a 3-hydroxy group;
a fatty triol with a 3-hydroxy group; a fatty tetrol with a 3-hydroxy group; an ω-hydroxy fatty acid with a 3-hydroxy group, an ω-carboxy fatty acid with a 3-hydroxy group, an ω-hydroxy fatty ester with a 3-hydroxy group, an ω-carboxy fatty ester with a 3-hydroxy group, an α,ω-fatty diacid with a 3-hydroxy group, an α,ω-fatty diester with a 3-hydroxy group, or an α,ω-fatty diol with a 3-hydroxy group, or a combination thereof;
the 3-oxo fatty acid or derivative thereof is a 3-oxo fatty acid; a 3-oxo fatty acid ester; a 3-oxo fatty alcohol; a 3-oxo fatty alcohol acetate ester; a 3-oxo fatty aldehyde; a 3-oxo fatty amine; a 3-oxo fatty amide; a 3-oxo fatty diol; a 3-oxo fatty triol; a 3-oxo fatty tetrol; an ω-hydroxy fatty acid with a 3-oxo group, an ω-carboxy fatty acid with a 3-oxo group, an ω-hydroxy fatty ester with a 3-oxo group, an ω-carboxy fatty ester with a 3-oxo group, an α,ω-fatty diacid with a 3-oxo group, an α,ω-fatty diester with a 3-oxo group, or an α,ω-fatty diol with a 3-oxo group, or a combination thereof; and
the trans-2-fatty acid or derivative thereof is a trans-2-fatty acid; a trans-2-fatty acid ester; a trans-2-fatty alcohol; a trans-2-fatty alcohol acetate; a trans-2-fatty aldehyde; a trans-2-fatty amine; a trans-2-fatty amide; a trans-2-fatty 1,3-diol; a trans-2-fatty diol; a trans-2-fatty triol; a trans-2-fatty tetrol; a trans-2-ω-hydroxy fatty acid, a trans-2-ω-carboxy fatty acid, a trans-2-ω-hydroxy fatty ester; a trans-2-ω-carboxy fatty ester; a trans-2-α,ω-fatty diacid; a trans-2-α,ω-fatty diester; or a trans-2-α,ω-fatty diol, or a combination thereof.

69. The modified biosynthetic pathway of any one of claims 60-68, wherein the modified biosynthetic pathways produces a fatty acid derivative composition comprising less than 50%, less than 45%, less than 40%, less than 35%, less than 30%, less than 25%, less than 20%, less than 15%, less than 10%, less than 5%, less than 4%, less than 3%, less than 2%, less than 1%, less than 0.5%, or less, by weight of the composition, of i) 3-hydroxy fatty acids and derivatives thereof; ii) trans-2-fatty acids and derivatives thereof; or iii) 3-oxo fatty acids and derivatives thereof; or a combination thereof.

70. The modified biosynthetic pathway of any one of claims 60-69 that is a fatty acid biosynthetic pathway.

71. A recombinant microbe, comprising the modified biosynthetic pathway of any one of claims 60-70.

72. The recombinant microbe of claim 71, wherein the recombinant microbe produces a composition comprising fatty acids and derivatives thereof, and comprising a reduced amount of 3-hydroxy fatty acids and derivatives thereof, and a reduced amount of 3-oxo fatty acids and derivatives thereof, as compared to a corresponding microbe that does not comprise the modified biosynthetic pathway.

73. Use of the recombinant microbe of any one of claims 1-40, 71, and 72; or the cell culture of claim 41; or the method of any one of claims 42-53; or the modified biosynthetic pathway of any one of claims 60-70, for the preparation of a fatty acid derivative, or a fatty acid derivative composition.

74. Use of the recombinant microbe of any one of claims 1-40, 71 and 72; or the cell culture of claim 41; or the method of any one of claims 42-53; or the modified biosynthetic pathway of any one of claims 60-70, for the preparation of a fragrance, flavor, pheromone, fuel, nutritional supplement, dietary supplement, pharmaceutical, or nutraceutical, ingredient or product, or a precursor thereof.

75. Use of the fatty acid, the fatty acid derivative composition, or the composition, of any one of claims 54-59, for the preparation of a fragrance, flavor, pheromone, fuel, nutritional supplement, dietary supplement, pharmaceutical, or nutraceutical, ingredient or product, or a precursor thereof.

76. A fragrance, flavor, pheromone, fuel, nutritional supplement, dietary supplement, pharmaceutical, or nutraceutical, ingredient or product, or a precursor thereof, prepared by the recombinant microbe of any one of claims 1-40, 71, and 72; or the cell culture of claim 41; or the method of any one of claims 42-53; or the modified biosynthetic pathway of any one of claims 60-70.

77. A fragrance, flavor, pheromone, fuel, nutritional supplement, dietary supplement, pharmaceutical, or nutraceutical, ingredient or product, or a precursor thereof, comprising the fatty acid, the fatty acid derivative composition, or the composition, of any one of claims 54-59.
